(19) United States
(12) Patent Application Publication
(10) Pub. No.: US 2017/0233817 A1
(43) Pub. Date: Aug. 17, 2017

(54) 16S RRNA SALIVA ANALYSIS UNVEILS MICROBIOME BIOMONITORS LINKED TO HUMAN PAPILLOMA VIRUS AND OROPHARYNGEAL SQUAMOUS CELL CARCINOMA

(71) Applicant: THE JOHNS HOPKINS UNIVERSITY, Baltimore, MD (US)

(72) Inventors: Rafael Guerrero-Preston, Baltimore, MD (US); David Sidransky, Baltimore, MD (US)

(21) Appl. No.: 15/128,439

(22) PCT Filed: Mar. 31, 2015
(86) PCT No.: PCT/US2015/023524

§ 371 Date: Sep. 23, 2016

Related U.S. Application Data
(60) Provisional application No. 61/972,675, filed on Mar. 31, 2014.
(60) Provisional application No. 61/975,169, filed on Apr. 4, 2014.
(60) Provisional application No. 62/133,584, filed on Mar. 16, 2015.

(51) Int. Cl. C12Q 1/68

(52) U.S. Cl. CPC C12Q 1/6886; C12Q 1/6874; C12Q 1/689; C12Q 2600/158; C12Q 2600/16

(57) ABSTRACT

The present invention relates to the field of cancer. More specifically, the present invention provides methods and compositions useful in the diagnosis and treatment of head and neck squamous cell carcinoma. As described herein, and particularly in reference to the figures, the present inventors have discovered that OTUs and several microbial communities at different taxonomic levels discriminates HNSCC from normal control samples, HPV+ and HPV− samples and pre- vs. post-surgical treatment samples. Appropriate diagnostic and treatment strategies can be employed based on the identification of the microbiota in patients' saliva.

### CROSS-REFERENCE TO RELATED APPLICATIONS

This application claims the benefit of U.S. Provisional Application No. 62/133,584, filed Mar. 16, 2015; U.S. Provisional Application No. 61/975,169, filed Apr. 4, 2014; and U.S. Provisional Application No. 61/972,675, filed Mar. 31, 2014; each of which are incorporated herein by reference in their entireties.

### STATEMENT OF GOVERNMENTAL INTEREST

This invention was made with government support under grant no. K01CA164092, awarded by the National Institutes of Health. The government has certain rights in the invention.

### FIELD OF THE INVENTION

The present invention relates to the field of cancer. More specifically, the present invention provides methods and compositions useful in the diagnosis and treatment of head and neck squamous cell carcinoma.

### INCORPORATION-BY-REFERENCE OF MATERIAL SUBMITTED ELECTRONICALLY

This application contains a sequence listing. It has been submitted electronically via EFS-Web as an ASCII text file entitled “P12973-04_ST25.txt.” The sequence listing is 3,030 bytes in size, and was created on Mar. 30, 2015. It is hereby incorporated by reference in its entirety.

### BACKGROUND OF THE INVENTION

The oral microbiome plays a critical role in the maintenance of a normal oral physiological environment and in development of oral diseases, including periodontal disease and tooth loss. Although little studied, the oral microbiome may also be important in cancer and other chronic diseases, through direct metabolism of chemical carcinogens and through systemic inflammatory effects. High-throughput technology provides the possibility of surveying microbial community structure at high resolution.

### SUMMARY OF THE INVENTION

The major approaches to cost-efficient high-throughput characterization of the human microbiome exploit the high variability in microbial 16S ribosomal RNA (rRNA) gene sequence, uniquely found in prokaryotes and considered as a barcode that can be used to identify specific microbes, characterizing the broad spectrum of both culturable and non-culturable organisms. Microbiome community profiles assessed by 16S rRNA pyrosequencing provide a broad spectrum of taxa identification, a distinct sequence-read record, and robust detection sensitivity. These results can be used to develop a saliva based diagnostic test for HNSCC. We amplified the 16S rRNA V3-V5 gene region of tumor and normal samples and performed 454 pyrosequencing. Briefly, DNA primers to highly conserved regions in the 16SrRNA V3-V5 gene region were designed for PCR amplification of DNA product, followed by DNA sequencing for characterization of microbial communities, including nonidentifiable types, based on DNA sequence in the highly variable inter-primer regions.

Accordingly, in one aspect, the present invention provides diagnostic and therapeutic strategies based on the microbiota identified in a patient's saliva. In certain embodiments, the present invention provides a method for identifying a human subject as having head and neck squamous cell cancer (HNSCC) comprising the steps of (a) obtaining nucleic acid from a saliva sample taken from the subject; (b) amplifying the 16S rRNA V3-V5 gene region of bacterial nucleic acid present in the nucleic acid of step (a); (c) sequencing the amplified DNA of step (b); (d) identifying the taxonomic levels of bacteria present in the saliva sample based on the sequences of step (c) and a comparison to taxonomic levels of bacteria present in a reference or control sample that correlates to normal mucosa; and (e) identifying the subject as having HNSCC or normal mucosa based on one or more of the following (i) enrichment in the observed species category of alpha-diversity estimators is indicative of normal mucosa; (ii) enrichment of Bacteriodetes Flavobacteria is indicative of normal mucosa; (iii) the presence of the genus Tannerella in the sample is indicative of normal mucosa; (iv) enrichment of Fusobacteriales Leptotrichiaceae is indicative of normal mucosa; (v) a higher number of operational taxonomic units (OTUs) is indicative of normal mucosa; (vi) a lower level of Aerococcaceae Abiotrophia is indicative of normal mucosa; (vii) at the family level, the taxon Fusobacteriales Leptotrichiaceae is dramatically enriched in normal mucosa; (viii) a threshold of 10 sequences assigned to Fusobacteriales Leptotrichiaceae distinguishes HNSCC from normal mucosa; (ix) the genus Tannerella was exclusively observed in normal mucosa; (x) threshold of 80 OTUs was enough to perfectly distinguish UPPP from OPSCC samples; (xi) 46 OTUs changed significantly in HNSCC patients (p<0.05) when compared to the controls mainly due to the loss of Neisseria and Aggregatibacter (Proteobacteria), Leptotrichia (Fusobacteria) and Veilonella (Firmicutes) with an increase in some Lactobacillus (Firmicutes); and (xii) within Bacteroidetes, Prevotella OTUs were found more abundant in control samples.

In particular embodiments, the primer set comprises the 357F/296R primer set. In a specific embodiment, the primer set comprises SEQ ID NO:2 and SEQ ID NO:3. In another specific embodiment, the comparison in identification step (d) can comprise a comparison to a library of 16s rRNA/rDNA bacterial gene sequences using analysis software. The software provides taxonomic classification of the relevant bacteria.

In another embodiment, a threshold of 80 OTUs is used in step (e)(v) to distinguish between normal from HNSCC. In a further embodiment, the alpha-diversity estimators of step (e)(i) comprises Chao2, ACE, Shannon and Simpson index. In yet another embodiment, a threshold of 10 sequences is used in step (e)(iv) to determine enrichment of Fusobacteriales Leptotrichiaceae.

In a specific embodiment, the subject is human papillomavirus (HPV) positive. In another specific embodiment, the subject is HPV negative. In particular embodiments, the methods further comprise the step of treating the subject with an appropriate treatment modality for HSNCC. In specific embodiments, the treatment modality is one or more of surgery (including laryngectomy, lymph node dissection, etc.), radiotherapy (external beam radiation therapy, intensity-modulated radiation therapy (IMRT), proton therapy, brachytherapy), and chemotherapy. In another embodiment, the treatment modality further comprises one or more of administering a cell cycle inhibitor, a PI3K inhibitor and/or a mTOR inhibitor.

In further embodiments, the subject is identified as having HNSCC and further comprising the step of (f) identifying the HNSCC subject as HPV+ or HPV− based on one or more of the following (i) at the class level, statistical depletion of Bacteroidetes Flavobacteria in HNSCC HPV+ samples relative to control samples; (ii) at the genus level, low-level presence of Aerococcaceae Abiotrophia distinguishes normal mucosa from OPSCC HPV+ samples; (iii) HPV+ samples are more diverse in terms of phylum, having unique Chloroflexi, Proteobacterial and Prevotella OTUs; (iv) HPV− samples have unique Actinobacterial OTUs that are lacking in the HPV+ samples; and (v) HPV+ samples enriched in the observed species category of alpha-diversity estimators relative to control samples.

In a more specific embodiment, the unique Actinobacterial OTUs of step (f)(iv) comprise Bifidobacteriaceae. In another embodiment, the alpha-diversity estimators of step (f)(v) comprise Chao1, ACE, Shannon and Simpson index. In particular embodiments, the method further comprises the step of treating the subject with an appropriate treatment modality for HSNCC. In a specific embodiment, the treatment modality is one or more of surgery (including laryngectomy, lymph node dissection, etc.), radiotherapy (external beam radiation therapy, intensity-modulated radiation therapy (IMRT), proton therapy, brachytherapy), and chemotherapy. In a further embodiment, the treatment modality comprises administering a cell cycle inhibitor to a HNSCC HPV− subject. In yet another embodiment, the treatment modality comprises administering a PI3K inhibitor and/or a mTOR inhibitor to a HNSCC HPV+ subject.

The present invention also provides methods for treating a patient for HNSCC HPV+/− based on the microbiota present in the patient's saliva. In a specific embodiment, the method comprises the step of administering an appropriate treatment to a patient identified as having HNSCC HPV+ based on the microbiota present in the patient's saliva. In another embodiment, a method comprises the step of administering an appropriate treatment to a patient identified as having HNSCC HPV− based on the microbiota present in the patient's saliva. The present invention also provides a method directed to ordering a diagnostic test to determine a patient's HNSCC HPV status based on the microbiota present in a saliva sample. The method can further comprise prescribing/administering/treating the patient based on the HNSCC HPV status.

The present invention also provides kits. The kits can be used to identify the microbiota present in a saliva sample obtained from a patient. The kit can comprise components for performing a PCR amplification of one, more or all of the nucleic acids described herein. In one embodiment, the kit comprises primers for amplifying the 16s rRNA/DNA V3-V5 regions of bacterial DNA. Other regions are contemplated and can be identified by one of ordinary skill in the art. Such regions can include, but are not limited to, V1-3, ITS1 and ITS2. In particular embodiments, the primers comprise the 357F/296R primer set. In a specific embodiment, the primers comprise SEQ ID NO:2 and SEQ ID NO:3. The primer can also comprise a non-complementary region (e.g., a barcode region). In another embodiment, the kit can also comprise a saliva collection/storage container. In specific embodiments, the kit comprises positive control DNA, negative control, and/or a master mix for performing PCR amplifications. In another embodiment, the kit comprises components for sequencing the amplified products. In a specific embodiment, the kit comprises a mix for forward/reverse sequencing of amplified PCR products. In certain embodiments, a separate PCR kit and a separate sequencing kit is provided. Alternatively, a kit can comprise components for both PCR amplification and sequencing. The kit can also comprise instructions for carrying out the amplification and/or sequencing protocols.

### BRIEF DESCRIPTION OF THE FIGURES

FIG. 1. is an analysis at the order level of the samples, using log-transformed proportions as the values.

FIG. 2. PCoA plot that uses the unweighted Unifrac distance metric, and displays clustering of samples colored by cancer and HPV status (OC HPV+ in blue, OC HPV− in greedn, Normal in red).

FIG. 3. A hierarchical clustering of samples at different taxonomic levels with a heatmap overly.

FIG. 4. A hierarchical clustering of samples at different taxonomic levels with a heatmap overly.

FIG. 5. A hierarchical clustering of samples at different taxonomic levels with a heatmap overly.

FIG. 6. A hierarchical clustering of samples at different taxonomic levels with a heatmap overly.

FIG. 7A. HPV+ OTUs. FIG. 7B. HPV− OTUs.

FIG. 8. 454 barcode mapping. Summarizes the 5′barcode mapping of samples and associated clinical metadata for both GS Junior runs.

FIG. 9. Phylum data.

FIG. 10. Class data.

FIG. 11. Taxonomic differences at the genus level according to the different sample types. Overall genus-level differences per sample type indicate a higher abundance of Streptococcus in the oropharynx and oral cavity samples compared to the normal samples, as well as Veilonella and Neiserraceae. These are not significant differences.

FIG. 12. Phylum-level histograms of samples organized according to histology and HPV status.

FIG. 13. Class-level histograms of samples organized according to histology and HPV status.

FIG. 14. Order-level histograms of samples organized according to histology and HPV status.

FIG. 15. Family-level histograms of samples organized according to histology and HPV status.

FIG. 16. Genus-level histograms of samples organized according to histology and HPV status.

FIG. 17. Alpha Rarefaction Curve Output chao1 Parameter for HNSCC and Normal epithelium samples.

FIG. 18. Alpha Rarefaction Curve Output Observed Species Parameter for HPV Histology. Alpha Rarefaction Curves (FIG. 17) showing richness (number of species) comparing normal (red) versus squamous cell carcinoma (blue). Interestingly, cancer results in a loss of bacterial species.

FIG. 19. Alpha diversity richness plots. Alpha diversity richness plots show higher richness values for Chao2, ACE, Shannon, and Simpson stats parameters in Normal epithelium when compared to HNSCC. HPV positive HNSCC samples have increased richness values for Chao1, ACE, Shannon, and Simpson stats parameters when compared to HPV negative samples.

FIG. 20. Beta Diversity PCoA by HNSCC Status. Principal Component Analyses PCoA shows a difference in the microbial communities between HNSCC and Normal samples. Additionally, HNSCC samples tend to cluster together according to HPV status (FIG. 21). Relating to this analysis, a clustered heatmap showing similarities between Significant OTUs of HNSCC and Normal Samples is shown on FIG. 31.

FIG. 21. Beta Diversity PCoA by OC-HPV Status. Beta Diversity PCoA show three mayor clusters showing distance between communities with Normal, HPV− negative HNSCC and HPV-positive HNSCC status. Three mayor clusters form metadata categories showing closer distances of communities showing correlation between OC and HPV status.

FIG. 22. Beta Diversity PCoA by HPV Status. PCoA plots do show microbial community differences according to HPV status as well as the communities show some differences according to the age range (FIG. 23).

FIG. 23. Beta Diversity PCoA by AGE RANGE.

FIG. 24. Beta Diversity PCoA by HNSCC Histology and Normal Tissue. There are no apparent differences between Oral Cavity HNSCC and Oropharynx HNSCC; however, when performing statistical analyses comparing HNSCC differences between Oral Cavity HNSCC and Oropharynx HNSCC the following taxonomic relative abundance differences were found (FIG. 25). Significant differences were also found according to Histology with 4 phyla changing significantly (Proteobacteria, Fusobacteria, Firmicutes and Bacteroidetes) (FIGS. 26, 27, 28 and 29). Maximum likelihood statistical significance tests (g tests) showed 46 significantly associated OTUs showing HISTOLOGY status metadata.

FIG. 25. Heatmap showing significantly different OTUS that change according to histology (Normal vs Oral Cavity SCC and Oropharynx SCC). Within the Phylum Proteobacteria, Genus Neisseria loses abundance for samples in Oral Cavity SCC and Oropharynx SCC. Genus Aggregatibacter is significantly less abundant as well for samples with HNSCC for both Oral Cavity and Oropharynx (FIG. 26).

FIG. 26. Significant different OTUs for the Proteobacteria Phylum according to histology (Normal vs Oral Cavity SCC and Oropharynx SCC).

FIG. 27. Significant different OTUs for the Fusobacteria Phylum according to histology (Normal vs Oral Cavity SCC and Oropharynx SCC). Within Phylum Fusobacteria, Genus Leptotrichia has lower abundance for samples of Oral Cavity (yellow) SCC and Oropharynx SCC.

FIG. 28. Significant different OTUS for the Firmicutes Phylum according to histology (Normal vs Oral Cavity SCC and Oropharynx SCC). Within the Firmicutes phylum, genus Lactobacillus is significantly more abundant for Oral Cavity SCC and Oropharynx SCC samples than control samples. OTUs within genus Veillonella are more abundant in control samples than in Oral Cavity SCC and Oropharynx SCC samples.

FIG. 29. Significant different OTUS for the Bacteroidetes Phylum according to histology (Normal vs Oral Cavity SCC and Oropharynx SCC). Genus Prevotella is more abundant in Control Samples and are significantly less abundant for Oral Cavity SCC and Oropharynx SCC samples.

FIG. 30. OTUS for the Actinobacteria Phylum according to histology (Normal vs Oral Cavity SCC and Oropharynx SCC). No major significant differences were observed for Actinobacteria taxa between samples.

FIG. 31. Significant OTUs from OTU network comparing Normal Cell Tissue (green) with HNSCC (red) Status. Clustering heatmap (done using the hclust algorithm with hierarchical cluster analysis) shows clustering between control (normal samples) and HNCSS samples, mainly due to the abundance of Lactobacillus, Streptococcus ad Parvimonas OTUs in HNCSS samples. In fact, The Heatmap shows OTUs that Genus Streptococcus, Citrobacter, Dialister, Fusobacterium, Lactobacillus, Leuconostoc, Parvimonas, Pseudomonas, Staphylococcus have a higher relative abundance for samples with HNSCC status. In contrast, Prevotella OTUs, Aggregatibacter, Granulicatella, Haemophilus, Lautropia, Leptotrichia, Neisseria, Oribacterium have a higher relative abundance for samples of the Control group. As in the heatmap of FIG. 31, a phylogenetic tree (not shown) for all Lactobacillus OTUs and their relative abundance shows that normal samples have much less Lactobacillus OTUs than squamous cell carcinoma.

FIG. 32. Barplot showing Significant OTU abundance Between Normal (Control), HNSCC Oral and HNSCC Oropharynx. FIG. 32 confirms the higher abundance of Streptococcus and Citrobacter in Oropharynx and oral cavity compared to normal samples.

FIG. 33. Oropharynx HNSCC HPV Negative Unique OTUs Taxa Summary Pie Chart.

FIG. 34. Oral Cavity HNSCC HPV Negative Unique OTUs Taxa Summary Pie Chart.

FIG. 35. Oropharynx HNSCC HPV Positive Unique OTUs Taxa Summary Pie Chart.

FIG. 36. Oral Cavity HNSCC HPV Positive Unique OTUs Taxa Summary Pie Chart.

FIG. 37. Normal (Control) HPV Negative Unique OTUs Taxa Summary Pie Chart.

FIG. 38. Barplot showing Significant OTUs at Phylum taxa level comparing relative abundance between Normal (Control) and HNSCC. FIG. 38 shows a higher abundance of Firmicutes in HSNSCC.

FIG. 39. Barplot showing Significant OTUs at Genus taxa level comparing relative abundance between Normal (Control) and HNSCC. Genus Veillonella, Streptococcus, Lactobacillus, Fusobacteria and Citrobacter show a higher on samples with HNSCC. Neisseria shows a higher abundance on Normal (control) samples.

FIG. 40. Significantly different OTUS that change according to HPV and HNSCC histology. When all sample types are compared according to HPV status, there are very few differences at the phylum level. This is also due that there is only 1 oral cavity HPV positive sample compare to the 11-25 samples of HPV status in the control and oropharynx (See Table 1 in the Examples Section).

FIG. 41. Heatmap showing Oral Cavity HNSCC and HPV Status Significant OTUs.

FIG. 42. Heatmap showing Oropharynx HNSCC Significant OTUs according to HPV status.

FIG. 43. Heatmap showing Normal Tissue Significant OTUs. Significant differences between oropharynx samples HPV+ and negative are mostly due to an abundance of Prevotella and Neisseria in HPV− (FIG. 42, lower sample clade) and Streptococcus, Rothia and Lactobacillus in HPV+ samples (upper clade)

FIG. 44. Richness and diversity estimates for each of the 7 patients that underwent serial sampling. For each sample, the HPV status is indicated. In bold is the first sample for the patient before surgery.

FIG. 45. Phyla-level taxonomic profiles for the serial samples indicating its HPV status.

FIG. 46. Genus-level taxonomic profiles for the serial samples indicating its HPV status. Patient HN 3022556 showed a lower abundance of Lactobacillus OTUs and a higher abundance of Streptococcus (1009) OTUs through time. HPV Status shifted from HPV+ to HPV−. 15 OTUs changed significantly (FIG. 47).

FIG. 47. Patient HN 3022556 Significant OTUs according to HPV Status. Patient HN 3717285 showed a shift in 5 OTUs to a higher abundance of Streptococcus OTU, higher abundance of Atopobium OTU and a lower abundance of Parvimonas and Fusobacterium OTU through time (FIG. 48). HPV Status did not change.

FIG. 48. Patient HN 3717285 Significant OTUs according to HPV Status. Patient HN 3724512 did not have a change in HPV status; however there was a lower abundance of OTU 1009, a Streptococcus through time. Interestingly, Streptococcus OUT 7179 was lost in sampling time 2 and the third and last sampling appeared again. A higher abundance of Fusobacterium and Prevotella OTUs. HPV Status shows no shifting. A total of 39 OTUs changed through sampling (FIG. 49).

FIG. 49. Patient HN 3724512 Significant OTUs according to HPV Status. Patient 3870084 was sampled 4 times. FIG. 36 shows the 38 OTUs that shifted according to the 4 sampling times. A shift to a lower abundance of Prevotella, Fusobacterium and an increase in Veillonella OTUs by sampling time 3. All samples were HPV negative.

FIG. 50. Patient HN 3870084 Significant OTUs according to HPV Status. Patient 4180455 had 18 OTUs changing significantly during the 3 sample time points, however HPV status remained positive. There was a shift to a lower abundance of Prevotella, Streptococcus (OTU 7179), Veillonella and Haemophilus OTUs and a higher abundance for Streptococcus OTU (*1009). This OUT, 1009 OUT, increased in abundance in HPV negative samples in patient 3022556.

FIG. 51. HN 4180455 Significant OTUs according to HPV Status. Sample HN 4471201 showed again an association of Streptococcus OUT 1009 with HPV negative status (FIG. 52). This OTU disappears in the following samples when HPV status of the patient changes to positive and OTU 7179 appears associated with HPV positive status. 13 OTUs change for patient 4471201.

FIG. 52. Patient HN 4471201 showing Significant OTUs according to HPV Status. As for patient 4679951, only 13 OTUs change per sampling time. This patient showed an association between Streptococcus OTU 7179 with HPV negative status, different from previous patients.

FIG. 53. Patient HN 4679951 showing Significant OTUs according to HPV Status. HN 3022556: Sample 01 was HPV+ with a Lower Streptococcus (1009) Abundance, Sample 03 was HPV− with Higher Streptococcus (1009) Abundance; HN 4471201: Sample 01 was HPV− with Higher Streptococcus (1009) Abundance, Sample 03 was HPV+ with a Lower Streptococcus (1009) Abundance; HN 4679951: Sample 01 was HPV+ with a Lower Streptococcus (7179) Abundance, Sample 03 was HPV− with Higher Streptococcus (7179) Abundance: both samples from patients HN 3022556 and HN 4471201 show same abundance shifting correlation between HPV Status and OTU No. 1009 Streptococcus. HN 4679951 shows the same pattern between HPV Status and OTU No. 7179 also a Streptococcus.

FIG. 54. Significant OTUs for a Subset of Oropharynx samples according to HPV status. No Significant clustering between HPV type and Oropharynx HNSCC Histology was observed.

FIG. 55. Significant OTUs associated with Oropharynx and HPV Negative Status. Streptococcus OTU 1009 is abundant in HPV− samples and OTU 7179 as well.

FIG. 56. Significant OTUs for Oropharynx and HPV Positive status. Streptococcus OTU 7179 is significantly associated with HPV+ samples of oropharynx and OTU 1009 is in much lower abundance confirming its relationship with HPV negative samples.

FIG. 57. Alpha diversity richness plots (subset Oropharynx Histology HNSCC). In oropharynx, HPV positive samples are more diverse (higher Shannon values) than HPV negative samples. The number of species (richness) is also higher in HPV positive samples except for a few HPV negative outliers.

FIG. 58. Significant OTUs Normal VS HNSCC by Phylum Taxa.

FIG. 59. Significant OTUs Normal VS HNSCC by Class Taxa.

FIG. 60. Significant OTUs Normal VS HNSCC by Order Taxa.

FIG. 61. Significant OTUs Normal VS HNSCC by Family Taxa.

FIG. 62. Significant OTUs Normal VS HNSCC by Genus Taxa.

Significant OTUs—oropharynx hpv (different taxonomic levels) g-test, FIGS. 63-67. From FIGS. 63067, we appreciate a significant association between Streptococcus (Lactobacillales) and cancer, namely in HPV positive samples.

FIG. 63. BDiv OTUs Oropharynx HPV Status by Phylum Taxa (all OTUs without g-test).

FIG. 64. Significant OTUs Oropharynx HPV Status by Phylum Taxa (only significant OTUs with g-test).

FIG. 65. BDiv OTUs Oropharynx HPV Status by Class Taxa (all OTUs without g-test).

FIG. 66. Significant OTUs Oropharynx HPV Status by Class Taxa (only significant OTUs with g-test).

FIG. 67. BDiv OTUs Oropharynx HPV Status by Order Taxa (all OTUs without g-test).

FIG. 68. Significant OTUs Oropharynx HPV Status by Order Taxa (only significant OTUs with g-test).

FIG. 69. BDiv OTUs Oropharynx HPV Status by Family Taxa (all OTUs without g-test).

FIG. 70. Significant OTUs Oropharynx HPV Status by Family Taxa (only significant OTUs with g-test).

FIG. 71. BDiv OTUs Oropharynx HPV Status by Genus Taxa (all OTUs without g-test).

FIG. 72. Significant OTUs Oropharynx HPV Status by Genus Taxa (only significant OTUs with g-test).

FIG. 73. HN series genus.

FIG. 74. Time series phyla.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

As described herein, and particularly in reference to the figures, the present inventors have discovered that OTUs and several microbial communities at different taxonomic levels discriminates HNSCC from normal control samples, HPV+ and HPV− samples and pre- vs. post-surgical treatment samples. Appropriate diagnostic and treatment strategies can be employed based on the identification of the microbiota in patients' saliva.

In certain embodiments, HNSCC can be distinguished from normal (e.g., UPPP) based on one or more of the following:

1. At the family level, the taxon Fusobacteriales Leptotrichiaceae is dramatically enriched in UPPP samples;

2. A threshold of 10 sequences assigned to Fusobacteriales Leptotrichiaceae distinguishes UPPP from OPSCC samples;

3. The genus Tannerella was exclusively observed in UPPP samples;

4. UPPP communities are significantly enriched in the observed species category of alpha-diversity estimators, i.e., HNSCC patients had a significant loss in richness and diversity (p<0.05) compared to the controls;

5. Raw number of OTUs was significantly higher in the UPPPP group; threshold of 80 OTUs was enough to perfectly distinguish UPPP from OPSCC samples.

6. 46 OTUs changed significantly in HNSCC patients (p<0.05) when compared to the controls mainly due to the loss of Neisseria and Aggregatibacter (Proteobacteria), Leptotrichia (Fusobacteria) and Veilonella (Firmicutes) with an increase in some Lactobacillus (Firmicutes);

7. Within bacteroidetes, Prevotella OTUs were found more abundant in control samples;

8. Longitudinal analyses (3 time periods) of samples taken before and after surgery revealed a reduction in the alpha diversity measure after surgery, together with an increase of this measure in patients that recurred;

In particular embodiments, the HPV status within HNSCC can be distinguished based on one or more of the following:

1. At the class level, statistical enrichment of Bacteroidetes Flavobacteria in UPPP samples relative to OPSCC HPV+ samples. Thus, the depletion of Bacteroidetes Flavobacteria is characteristic of the OPSCC HPV+ samples in general;

2. At the genus level, low-level presence of Aerococcaceae Abiotrophia distinguishes UPPP samples from OPSCC HPV+ samples.

3. The HPV+ samples are more diverse in terms of phylum, having unique Chloroflexi and Proteobacterial OTUs as well as Prevotella. The HPV− samples have unique Actinobacterial OTUs that are lacking in the HPV+ ones, namely, Bifidobacteriaceae.

4. HPV positive samples were more diverse (higher Shannon values and richness) than HPV negative samples.

### I. Definitions

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

The term “cancer” or “tumor” is used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

“Chemotherapeutic agent” means a chemical substance, such as a cytotoxic or cytostatic agent, that is used to treat a condition, particularly cancer.

As used herein, “cancer therapy” and “cancer treatment” are synonymous terms.

As used herein, “chemotherapy” and “chemotherapeutic” and “chemotherapeutic agent” are synonymous terms.

As used herein, a “cell-cycle gene” is a gene whose activity affects regulation of the cell cycle, or whose expression levels vary periodically with the cell-cycle.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantially identical,” as used herein, refers to an identity or homology of at least 75%, at least 80%, at least 85%, at least 90%, 91%, 92%, 93%, 94%, 95%, 96%, 97%, 98%, 99%, or more.

“Likely to” or “increased likelihood,” as used herein, refers to an increased probability that an item, object, thing or person will occur. Thus, in one example, in certain embodiments, a HNSCC patient who is HPV+ is likely to be more sensitive to radiation and chemotherapies. In another embodiments, a HNSCC patient who is HPV− may be likely to respond to treatment with a cell cycle inhibitor, such as a CDK inhibitor, i.e., has an increased probability of responding to treatment with the cell cycle inhibitor CDK inhibitor relative to a reference subject or group of subjects.

“Unlikely to” refers to a decreased probability that an event, item, object, thing or person will occur with respect to a reference. Thus, a subject that is unlikely to respond to a particular treatment modality, has a decreased probability of responding to such treatment relative to a reference subject or group of subjects.

“Sequencing” a nucleic acid molecule requires determining the identity of at least 1 nucleotide in the molecule. In certain embodiments, the identity of less than all of the nucleotides in a molecule are determined. In other embodiments, the identity of a majority or all of the nucleotides in the molecule is determined.

“Next-generation sequencing or NGS or NG sequencing” as used herein, refers to any sequencing method that determines the nucleotide sequence of either individual nucleic acid molecules (e.g., in single molecule sequencing) or clonally expanded proxies for individual nucleic acid molecules in a highly parallel fashion (e.g., greater than 10.sup.5 molecules are sequenced simultaneously). In one embodiment, the relative abundance of the nucleic acid species can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, incorporated herein by reference. Next generation sequencing can detect a variant present in less than 5% of the nucleic acids in a sample.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of cancer. Moreover, a sample obtained from a patient can be divided and only a portion may be used for diagnosis. Further, the sample, or a portion thereof, can be stored under conditions to maintain sample for later analysis. The definition specifically encompasses solid tissue samples such as a biopsy specimen or tissue cultures or cells derived therefrom and the progeny thereof. In other embodiments, the term sample includes blood and other liquid samples of biological origin (including, but not limited to, peripheral blood, serum, plasma, cerebrospinal fluid, urine, saliva, stool and synovial fluid). In particular embodiments, a sample comprises a saliva sample.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry. In certain embodiments, a sample comprises an optimal cutting temperature (OCT)-embedded frozen tissue sample.

The terms “specifically binds to,” “specific for,” and related grammatical variants refer to that binding which occurs between such paired species as enzyme/substrate, receptor/agonist, antibody/antigen, nucleic acid/complement and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs.

“Statistically significant” means that the alteration is greater than what might be expected to happen by chance alone. Statistical significance can be determined by any method known in the art. For example, statistical significance can be determined by p-value. The p-value is a measure of probability that a difference between groups during an experiment happened by chance. For example, a P-value of 0.01 means that there is a 1 in 100 chance the result occurred by chance. The lower the P-value, the more likely it is that the difference between groups was caused by, e.g., treatment. An alteration is considered to be statistically significant if the P-value is at least 0.05. Preferably, the P-value is 0.04, 0.03, 0.02, 0.01, 0.005, 0.001 or less. In particular embodiments, enrichment/depletion of taxonomic levels of bacteria can be statistically significant.

Various methodologies of the instant invention include a step that involves comparing a value, level, feature, characteristic, property, etc. to a “suitable control,” referred to interchangeably herein as an “appropriate control,” a “control sample” or a “reference.” A “suitable control,” “appropriate control,” “control sample” or a “reference” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control cell, organ, or patient, exhibiting, for example, a normal phenotype. In another embodiment, a “suitable control” or “appropriate control” is a value, level, feature, characteristic, property, ratio, etc. determined prior to performing a therapy (e.g., cancer treatment) on a patient. In yet another embodiment, a microbiome profile can be determined prior to, during, or after administering a therapy into a cell, organ, or patient. In a further embodiment, a “suitable control,” “appropriate control” or a “reference” is a predefined value, level, feature, characteristic, property, ratio, etc. A “suitable control” can be a profile or pattern of levels/ratios of a bacteria of the present invention that correlates to a cancer and/or HPV status, to which a patient sample can be compared. The patient sample can also be compared to a negative control.

### II. Methods to Identify Microbial Nucleic Acids

Many methods for identifying the bacteria present in patient saliva samples via 16S rRNA nucleic acid expression are contemplated. Any reliable, sensitive, and specific method can be used. In particular embodiments, the microbial nucleic acid is amplified and sequenced.

Specific changes in microbiota can be detected using various methods including, but not limited to, quantitative PCR and high-throughput sequencing methods which detect over- and under-represented genes in the total bacterial population (e.g., 454-sequencing for community analysis), or transcriptomic or proteomic studies that identify lost or gained microbial transcripts or proteins within total bacterial populations. See, e.g., Eckburg et al., Science, 2005, 308:1635-8; Costello et al., Science, 2009, 326:1694-7; Grice et al., Science, 2009, 324:1190-2; Li et al., Nature, 2010, 464: 59-65; Bjursell et al., Journal of Biological Chemistry, 2006, 281:36269-36279; Mahowald et al., PNAS, 2009, 14:5859-5864; Wikoff et al., PNAS, 2009, 10:3698-3703.

Many methods exist for amplifying nucleic acid sequences. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR). See, e.g., Chen et al., 33(20) NUCL. ACIDS RES. e179 (2005).

A typical PCR reaction comprises multiple amplification steps or cycles that selectively amplify target nucleic acid species including a denaturing step in which a target nucleic acid is denatured; an annealing step in which a set of PCR primers (forward and reverse primers) anneal to complementary DNA strands; and an extension step in which a thermostable DNA polymerase extends the primers. By repeating these steps multiple times, a DNA fragment is amplified to produce an amplicon, corresponding to the target DNA sequence. Typical PCR reactions include about 20 or more cycles of denaturation, annealing, and extension. In many cases, the annealing and extension steps can be performed concurrently, in which case the cycle contains only two steps. Because mature mRNA are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than about 15, fewer than about 20, fewer than about 25, fewer than about 30, or fewer than about 35 nucleotides in length. In additional embodiments, a primer is at least about 35 nucleotides in length.

In a further embodiment, a forward primer can comprise at least one sequence that anneals to target nucleic acid sequence and alternatively can comprise an additional 5′ non-complementary region (e.g., a barcode primer). In another embodiment, a reverse primer can be designed to anneal to the complement of a reverse transcribed mRNA. The reverse primer may be independent of the target nucleic acid sequence, and multiple target nucleic acid sequences may be amplified using the same reverse primer. Alternatively, a reverse primer may be specific for a target nucleic acid.

In some embodiments, two or more microbial nucleic acid sequences are amplified in a single reaction volume. One aspect includes multiplex PCR (e.g., q-PCR, such as qRT-PCR), which enables simultaneous amplification and quantification of at least two nucleic acid sequences of interest in one reaction volume by using more than one pair of primers and/or more than one probe. The primer pairs comprise at least one amplification primer that uniquely binds each mRNA, and the probes are labeled such that they are distinguishable from one another, thus allowing simultaneous quantification of multiple target nucleic acid sequences. Multiplex qRT-PCR has research and diagnostic uses including, but not limited, to detection of target nucleic acid sequences for diagnostic, prognostic, and therapeutic applications.

The qRT-PCR reaction may further be combined with the reverse transcription reaction by including both a reverse transcriptase and a DNA-based thermostable DNA polymerase. When two polymerases are used, a “hot start” approach may be used to maximize assay performance. See U.S. Pat. No. 5,985,619 and U.S. Pat. No. 5,411,876. For example, the components for a reverse transcriptase reaction and a PCR reaction may be sequestered using one or more thermoactivation methods or chemical alteration to improve polymerization efficiency. See U.S. Pat. No. 6,403,341; U.S. Pat. No. 5,550,044; and U.S. Pat. No. 5,413,924.

### III. Treatment Modalities

As described herein, patients can be treated with an appropriate modality based on the identified microbiota. A skilled physician can readily determine treatment strategy, which can include, but is not limited to, surgery (including laryngectomy, lymph node dissection, etc.), radiotherapy (external beam radiation therapy, intensity-modulated radiation therapy (IMRT), proton therapy, brachytherapy), and chemotherapy.

The human papillomavirus (HPV) has been shown to cause a subset of head and neck cancers (HNC), especially the squamous cell carcinoma of the oropharynx. HPV-associated HNC has a distinct clinical profile from that of HPV-unrelated oropharyngeal cancer. It presents in younger age and more likely male patients, who are less likely to have a history of tobacco or alcohol abuse. Compared to HPV-unrelated HNC, HPV-associated HNC is also associated with a more favorable prognosis, likely due to its higher sensitivity to current radiation and chemotherapies. In recent decades, the incidence of HPV-associated HNC has been increasing rapidly, probably attributable to increasing high risk sexual behaviors. Therefore, to better manage newly diagnosed HNC, it is currently recommended to determine HPV status in the tumor by the National Comprehensive Cancer Network (NCCN) guidelines.

In some embodiments, patients having HNSCC who are also HPV− can be treated with a drug that targets a cell cycle gene or a gene or protein that functions downstream of the cell cycle gene. For example, a HNSCC subject with an HPV− status can be treated with a CDK (cyclin dependent kinase) inhibitor, which will target CDK proteins overexpressed due to a CDKN2A or CDKN2B loss-of-function mutation, such as a CDKN2A or CDKN2B deletion.

In other embodiments, patients who are HPV+ may be less likely to respond to a treatment with a drug that targets a cell cycle gene, or a gene or protein that functions downstream of the cell cycle gene. For example, a HNSCC subject with an HPV+ status can be treated with a drug other than a CDK (cyclin dependent kinase) inhibitor, or a CCND1 inhibitor. The HPV+ HNSCC patient can alternatively be treated with a PI3K inhibitor and/or an mTOR inhibitor. Thus, evaluation of HPV-status in a subject with head and neck cancer can be used to evaluate cancer responsiveness.

In head and neck cancer (HNC), Human Papilloma Virus (HPV)-negative disease is usually associated with smoking and alcohol use and relatively poor survival. In certain embodiment, therapy includes cisplatin for 3 cycles, where each cycle is 21 days, combined with daily radiotherapy. In other embodiments, therapy can include weekly cisplatin with daily radiotherapy. In further embodiments, induction regimens can involve 2- or 3-chemotherapy agents followed by chemoradiation. Within HPV-negative HNC is a unique subpopulation of patients who present with oral cavity tumors, whose incidence has increased in recent years. It is important for clinicians to recognize that these oral cavity tumors primarily affect the tongue and occur predominantly in women. Surgery is the primary treatment, which needs to be performed adequately from the beginning. Some clinicians are hesitant to remove too much of a patient's tongue due to concern of worsening function, so they opt for radiation or radiation combined with chemotherapy, which evidence demonstrates are inferior alternatives to surgery.

Currently HPV+ OPC are treated similarly to stage-matched and site-matched unrelated OPC. However less intensive use of radiotherapy or chemotherapy, as well as specific therapy, can be prescribed. In certain embodiments, HPV+ HNC patients can benefit better from radiotherapy and concurrent cetuximab treatment than HPV− HNC patients receiving the same treatment.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Analysis of 16S rRNA Amplicon Sequences Associated with Oral Cancer and HPV Infection

As described herein, microbiota markers able to distinguish HPV positive from HPV negative Head and Neck Squamous Cell Carcinoma (HNSCC) cases, and also from normal patients provide improvement in treatment methods, reduction in treatment cost and improvement in survival rates. In the present study we compared the microbiota present in saliva DNA obtained from HPV positive and HPV negative oropharyngeal cancer (OPSCC) patients to saliva DNA obtained from uvulopalatopharyngoplasty (UPPP) patients, used as normal oral mucosa controls. PCR amplification of the 16S rRNA V3-V5 gene region was performed using the 357F/926R primer set. Amplified fragments were multiplexed on the Roche/454 GS Junior platform. Data were screened for chimeric sequences and contaminant chloroplast DNA after pre-processing. Passing sequences were characterized for diversity and taxonomic composition using the QIIME and R.

Bacteroidetes, Firmicutes, and Proteobacteria dominated the microbiome in our sample set, with less frequent presence of Actinobacteria and Fusobacteria members. Moving to lower taxonomic levels, the most abundant genera observed were Streptococcus, Prevotella and Veillonella. We found statistically significant associations with the alpha-diversity estimators. The raw number of operational taxonomic units (OTUs) was significantly higher in the UPPP group (p=0.02). In fact, a threshold of 80 OTUs was enough to perfectly distinguish UPPP from OPSCC samples. At the phylum level, we did not detect a significant difference in taxa using the Mann-Whitney test, but at lower levels statistically significant differences were detected. At the family level, the taxon Fusobacteriales Leptotrichiaceae is dramatically enriched in the UPPP samples (p=0.02), and is present in very high numbers in all UPPP samples. A threshold of 10 sequences assigned to Fusobacteriales Leptotrichiaceae also perfectly distinguished UPPP from OPSCC samples. At the genera level we found that the genus Tannerella was exclusively observed in UPPP samples (p=0.03). Examining the alpha-diversity estimators, we saw that UPPP communities are significantly enriched in the observed species category (p<0.001).

Comparing HPV status within the OPSCC samples, we found no differences between HPV+/− groups in the alpha-diversity estimators or phylum level abundances. However at the class level, we observed a statistical significant enrichment of the taxon Bacteroidetes Flavobacteria in the UPPP samples relative to OPSCC HPV+ samples (p=0.03). The depletion of Bacteroidetes Flavobacteria is characteristic of the OPSCC HPV+ samples in general. At the genus level, we observed that low-level presence of Aerococcaceae Abiotrophia completely distinguishes the UPPP samples from OPSCC HPV+ (p=0.02).

Our results suggest that the microbial diversity and taxonomic composition of the oral microbiota may be useful diagnostic and early detection biomonitors for HNSCC. Specifically, the loss of depletion of Bacteroidetes Flavobacteria in OPSCC HPV+ samples can be used as an easily quantifiable biomonitor of HPV+ in saliva. Furthermore, Aerococcaceae Abiotrophia distinguishes normal UPPP patients from OPSCC HPV+ in saliva. In addition, a threshold of 80 OTUs was enough to perfectly distinguish UPPP from OPSCC samples. A threshold of 10 sequences assigned to Fusobacteriales Leptotrichiaceae also perfectly distinguished UPPP from OPSCC samples. The genus Tannerella was exclusively observed in UPPP samples. Examining the alpha-diversity estimators, we saw that UPPP communities are significantly enriched in the observed species category. The combined use of these six biomonitors may be used as a robust diagnostic test for HNSCC in saliva. In sum, these results may provide the critical foundation for the identification of bacterial indicators of carcinogenesis in HNSCC and, in turn, suggest strategies for more effective diagnosis and treatment.

### Materials and Methods

The purpose of this example is to describe a series of preliminary results associated with the microbial diversity and taxonomic composition of human oral microbiota associated with oral cancer (OC) and/or infection of human papilloma virus (HPV). PCR amplification of the 16S rRNA V3-V5 gene region was performed for each sample using the 357F/926R primer set. Amplified fragments were sequenced in multiplex on two runs of the Roche/454 GS Junior platform.

Reads output by the sequencer were demultiplexed using 5′ barcodes, trimmed of forward and reverse primer sequences, filtered for length and quality, and corrected for homopolymer errors. The resulting high-quality dataset was then screened for chimeric sequences and contaminant chloroplast DNA.

Passing sequences were characterized for diversity and taxonomic composition using the QIIME and R packages. To begin, sequences were clustered into operational taxonomic units (OTUs) using UCLUST with a 95% identity threshold. Taxonomic assignment was performed using the RDP classifier (trained by a customized version of the comprehensive GreenGenes database, release v.13-05) with a minimum confidence threshold of 0.80. Here, we highlight the results of this initial analysis, with full data presented in the supplementary spreadsheet provided with this report. Results below are organized by the corresponding tab names in the Excel spreadsheet (RGP_Results_2013-09.xlsx).

454 Barcode Mapping: This tab summarizes the 5′ barcode mapping of samples and associated clinical metadata for both GS Junior runs. Run H4C0D1Q01 contained 8 multiplexed samples (2 Normal, 3° C. HPV+, 3° C. HPV−). Run IAJNLYC02 included four multiplex samples (1 Normal, 2° C. HPV+, 1° C. HPV−).

Preprocessing Stats: This tab summarizes the preprocessing steps for each run, which included quality filtering, error-correction, and chimera removal. Overall there were no major issues with preprocessing; loss of sequences due to chimeras was within the expected range, and there were no chloroplasts detected in the entire dataset.
The average final read length was 491 bp.
The average number of reads per sample was 11,907.
The minimum number of reads per sample was 3,594—This is excellent minimum coverage for multiplexed 454. Typically I need to throw out a few samples that end up having very low coverage, but not in this case.
The total final reads after preprocessing was 142,887.

Study Metadata: This tab shows the metadata that was used in my analysis after preprocessing. In all, we had three normal samples, five OC HPV+, and four OC HPV-samples.

*raw: The next set of tabs in the spreadsheet (Phylum raw, Class raw, Order raw, Family raw, Genus raw, Species raw, OTU raw) shows the total counts of 16S sequences associated with each taxon at their respective taxonomic levels. I've also included a stacked histogram with some of the tables to provide a better sense of the distribution of these taxa.

Phylum raw: This tab shows the raw counts of sequences assigned at the phylum level across samples. We find that oral communities in general are dominated by Bacteroidetes, Firmicutes, and Proteobacteria, with less frequent presence of Actinobacteria and Fusobacteria members. Moving to lower taxonomic levels, such as the tab Genus raw, we see the most abundant genera tended to be Streptococcus, Prevotella and Veillonella.

OTU raw: This tab provides the operational taxonomic units (OTUs) generated by UCLUST and their corresponding taxonomic assignment by the RDP classifier. This data serves as the basis for the other *raw tabs described above as well as the alpha- and beta-diversity analyses described below. A total of 303 OTUs were generated from the full dataset.

A note on subsampling: After considering the raw count data in full above, we typically perform subsampling of each community to an equivalent depth, in this case, 3,594 sequences per sample. All results described below are based on the subsampled data, which will help to mitigate biases due to differences in sampling depth. This explains why the text “even3594” is found throughout the remaining spreadsheet tabs.

*vs*: The final set of tabs in the spreadsheet provide results of statistical comparisons of groups of interest at various taxonomic levels, and, for multiple ecological diversity estimators. For each group comparison, we computed a variety of significance tests including the Metastats methodology, Mann-Whitney, Fisher's exact test, and the Negative Binomial test (from the DESeq package). The data in each tab is presented as the mean, variance, and standard error of each group, followed by the significance testing results. Additionally, the raw data used as input is also provided in the right-most columns.

Alpha-diversity metrics that were computed included: the raw number of OTUs per sample, Chao1 estimator, Good's coverage statistic, Shannon entropy, and the reciprocal Simpson index. These results are provided in the spreadsheets above the taxonomic specific results. Significance results are sorted by Mann-Whitney (M-W) p-values (from lowest to highest), but in some cases other significance tests may be more appropriate (e.g., Metastats or Negative Binomial for small group numbers).

### Results

Norm vs. OC: Comparing the three Normal samples to the nine OC samples, we find statistically significant results in the alpha-diversity estimators. In particular, the raw number of OTUs (observed_species) is significantly higher in the Normal group (M-W P=0.016), as is the PD_whole_tree diversity measure (M-W P=0.036). In fact a threshold of 80 OTUs is enough to perfectly distinguish Normal from OC samples.

At the phylum level, we do not detect a significant difference in taxa using the Mann-Whitney test, but at lower levels statistically significant differences are detected. For example, at the family level, the taxon Fusobacteriales_Leptotrichiaceae is dramatically enriched in the Normal samples (˜59-fold on average; M-W P=0.014), and is present in very high numbers for all Normal samples. A threshold of 10 sequences assigned to Fusobacteriales_Leptotrichiaceae also perfectly distinguishes Normal and OC samples.

OC HPV neg vs. OC HPV pos: Comparing HPV status within the OC samples, we find no differences between HPV+/− groups in the alpha-diversity estimators or phylum level abundances. However at the class level, the taxon Bacteroidetes Flavobacteria appears significantly enriched in the HPV− group (˜123-fold on average, M-W P=0.018). This is particularly striking when one views the raw count data in the right-most columns. In fact we can completely distinguish the two groups using a threshold of 5 sequences assigned to Flavobacteria. This distinguishing ability is also true for the Burkholderiales taxon.

Moving to the genus level, we see the genus Capnocytophaga (a member of Flavobacteria) can be largely attributed to the Flavobacteria enrichment in the HPV− samples.

Norm vs. OC HPV pos: Comparing the Normal oral communities to the OC HPV+ group, the Mann-Whitney test suffers from a loss of statistical power due to smaller group sizes, but we still observe a statistical enrichment of Bacteroidetes Flavobacteria in the Normal samples relative to OC HPV+ samples (M-W P=0.0324). The depletion of Bacteroidetes_Flavobacteria is characteristic of the OC HPV+ samples in general.

At the genus level, we find that low-level presence of Aerococcaceae_Abiotrophia completely distinguishes the Normal samples (M-W P=0.017) from OC HPV+. This was also true for Burkholderiaceae_Lautropia.

Due to the loss of statistical power here (n=3 vs. n=5), the significance results associated with the Negative Binomial or Metastats test may be more appropriate for this comparison.

Norm vs. OC HPV neg: Comparing the Normal oral communities to the OC HPV− group, we find multiple genera with moderately significant M-W P-values. In particular, the genus Tannerella is exclusively observed in the Normal samples (P=0.032). Examining the alpha-diversity estimators, we see that using the nonparametric difference test, Normal oral communities are significantly enriched in the observed_species category (P=0.0002).

Additional Materials

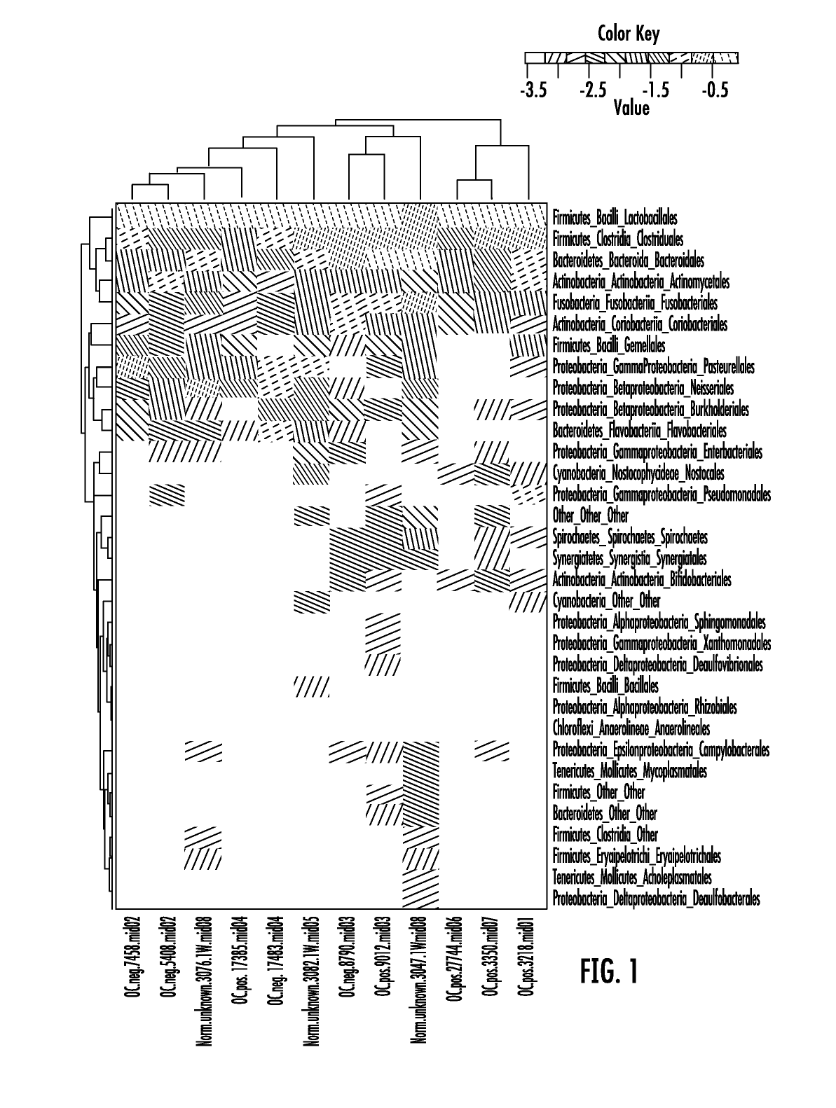

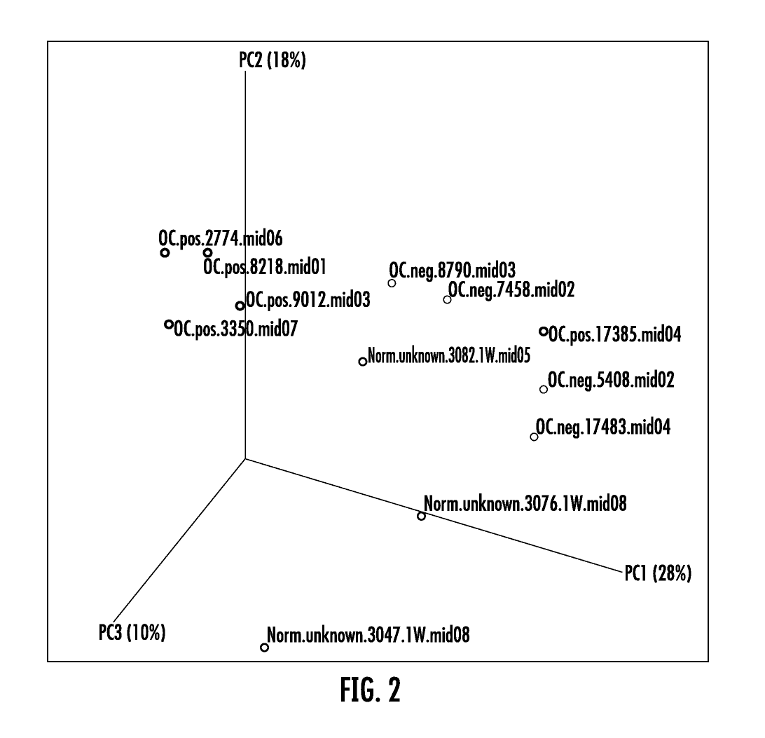

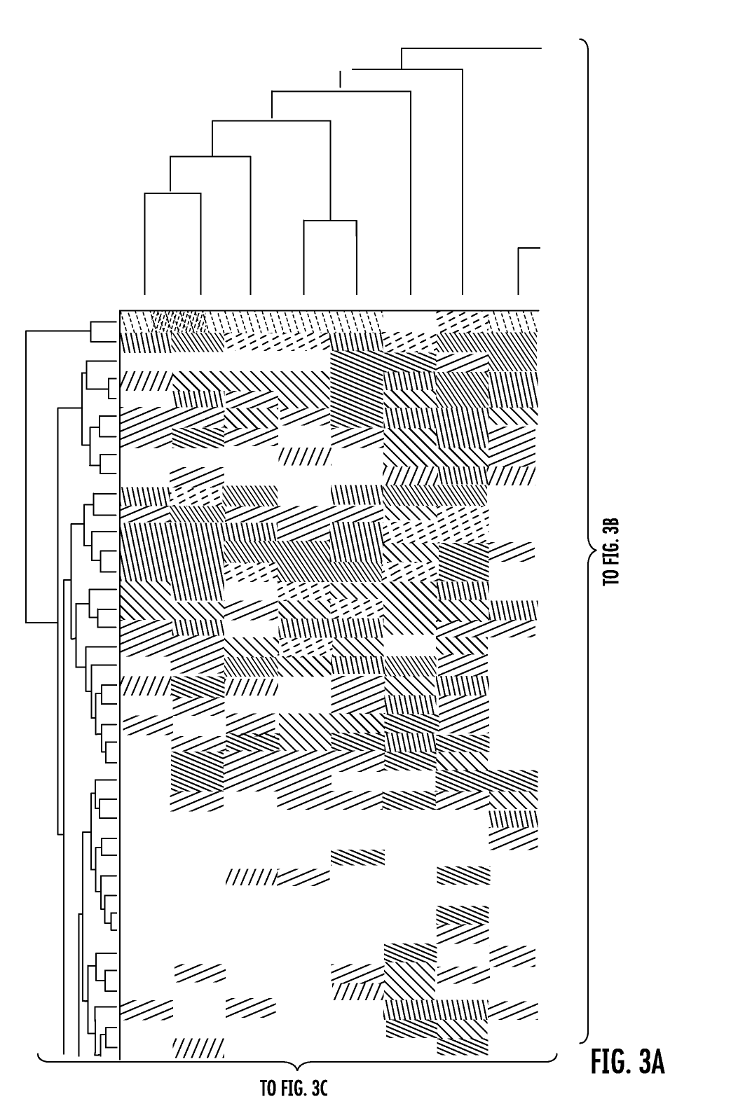

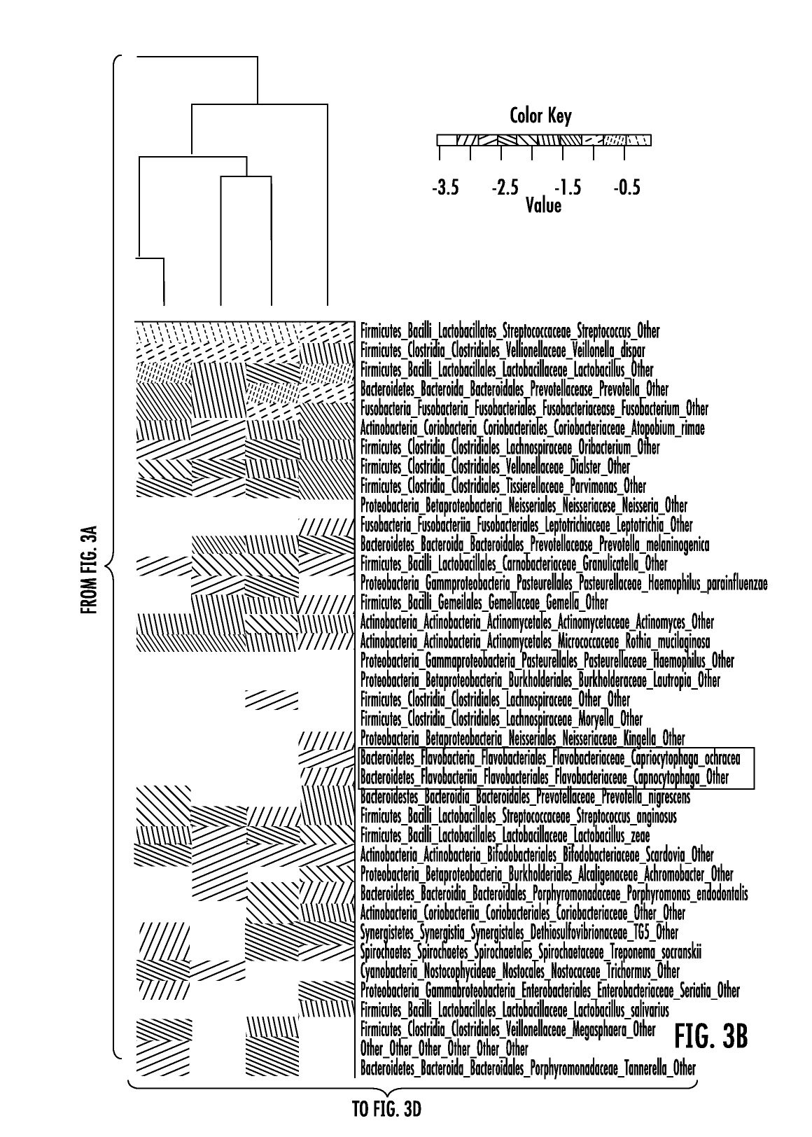

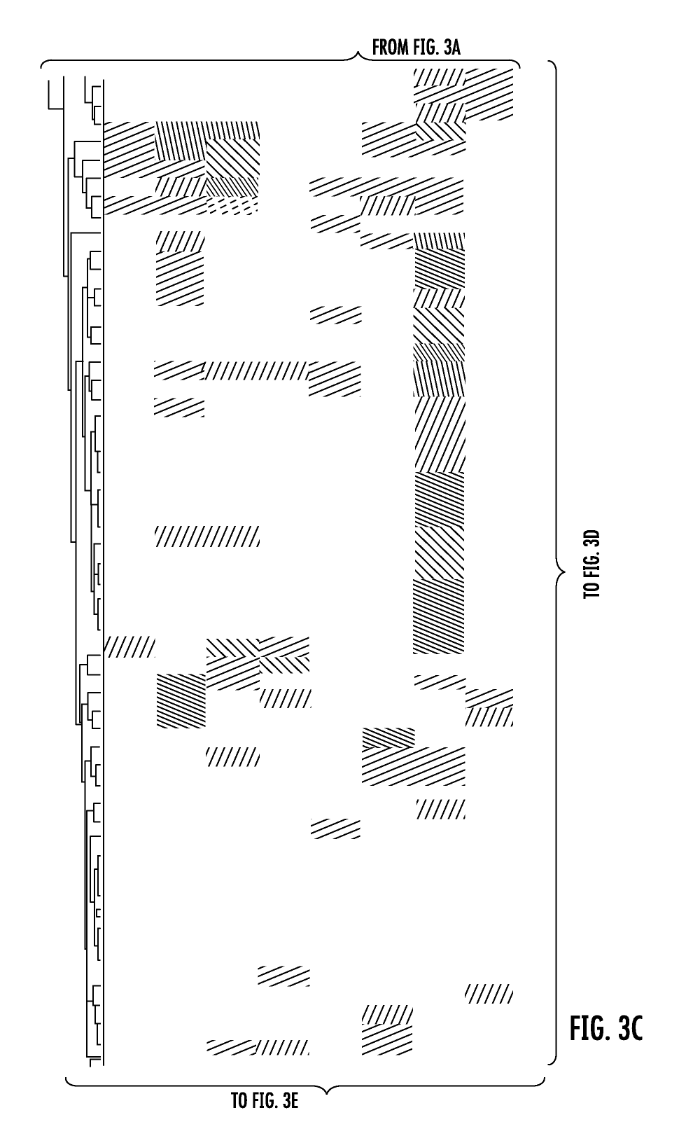

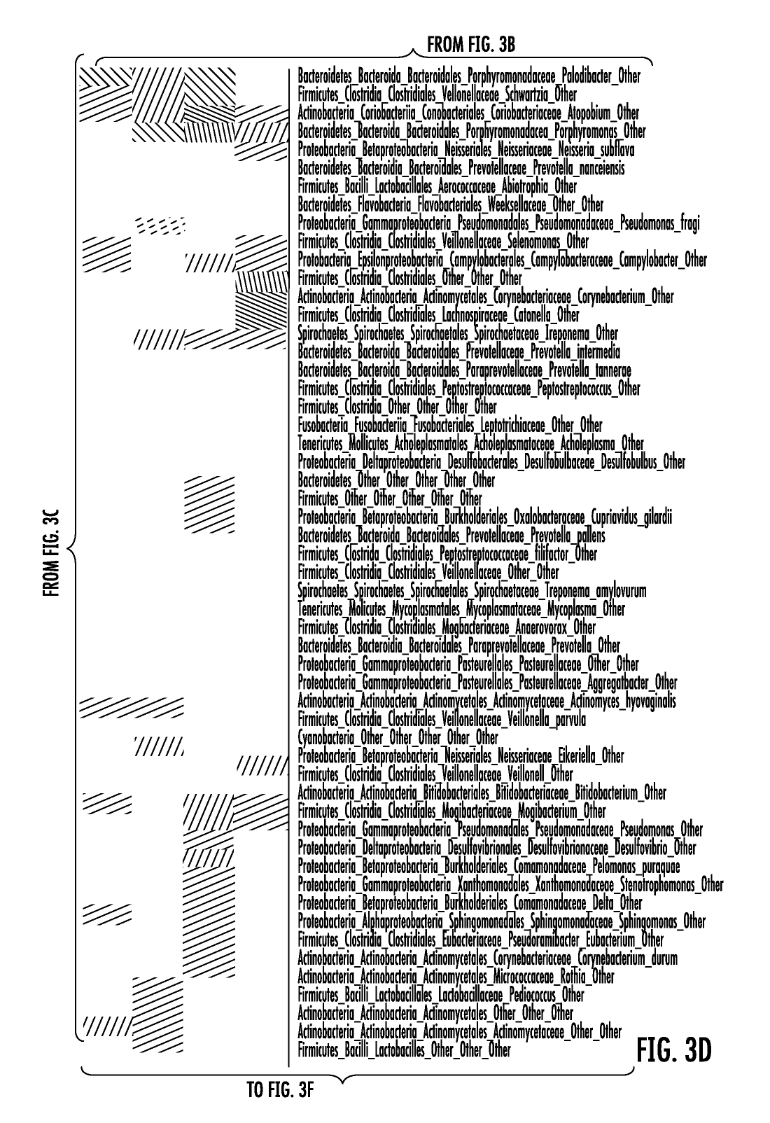

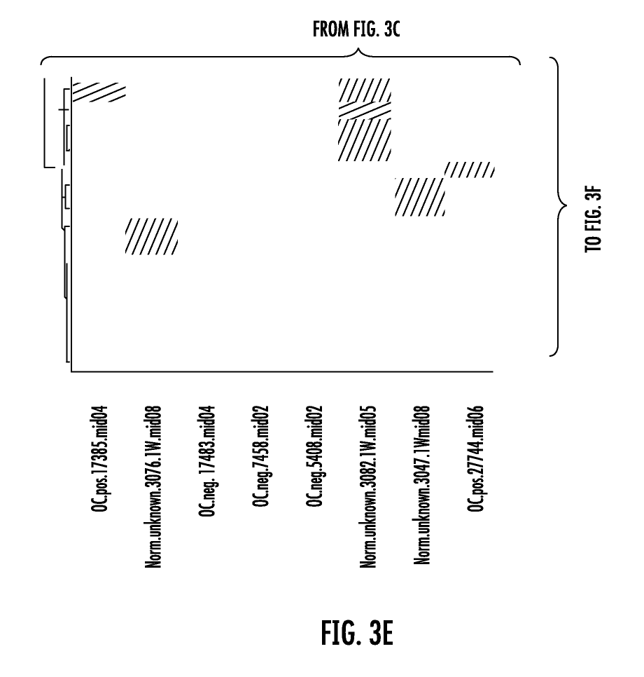

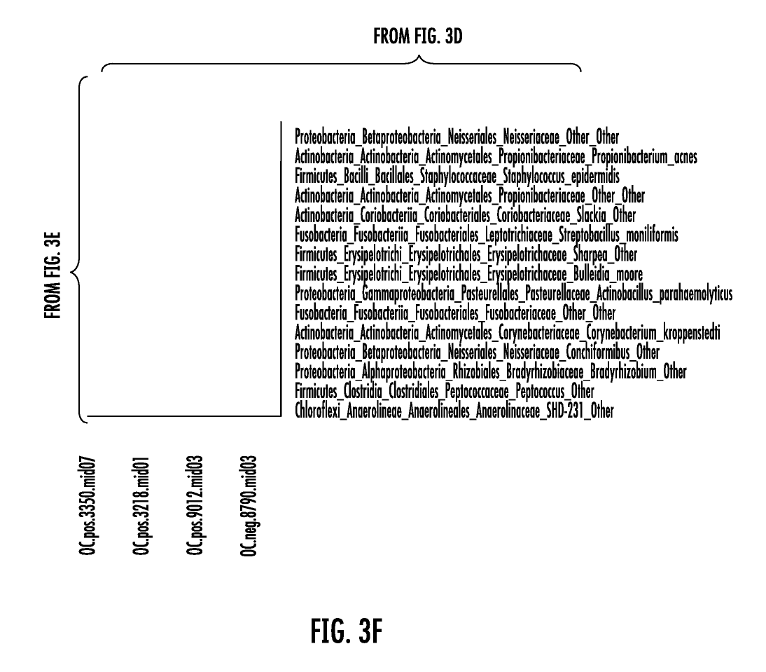

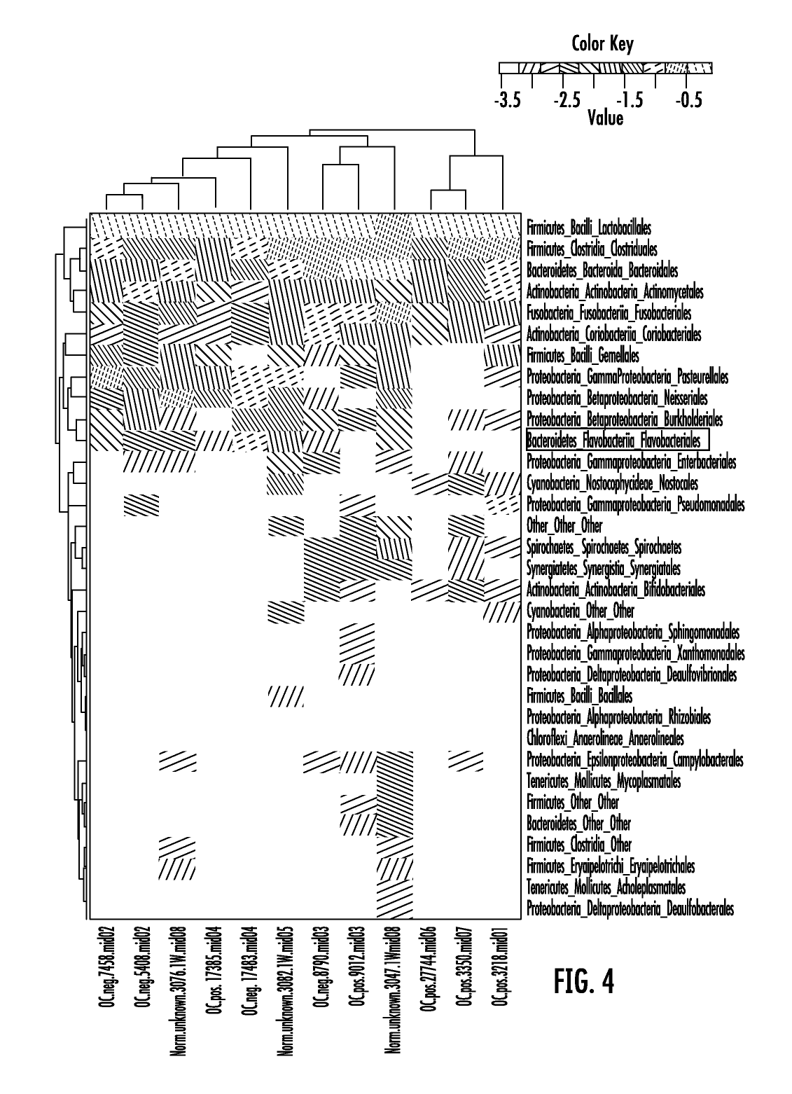

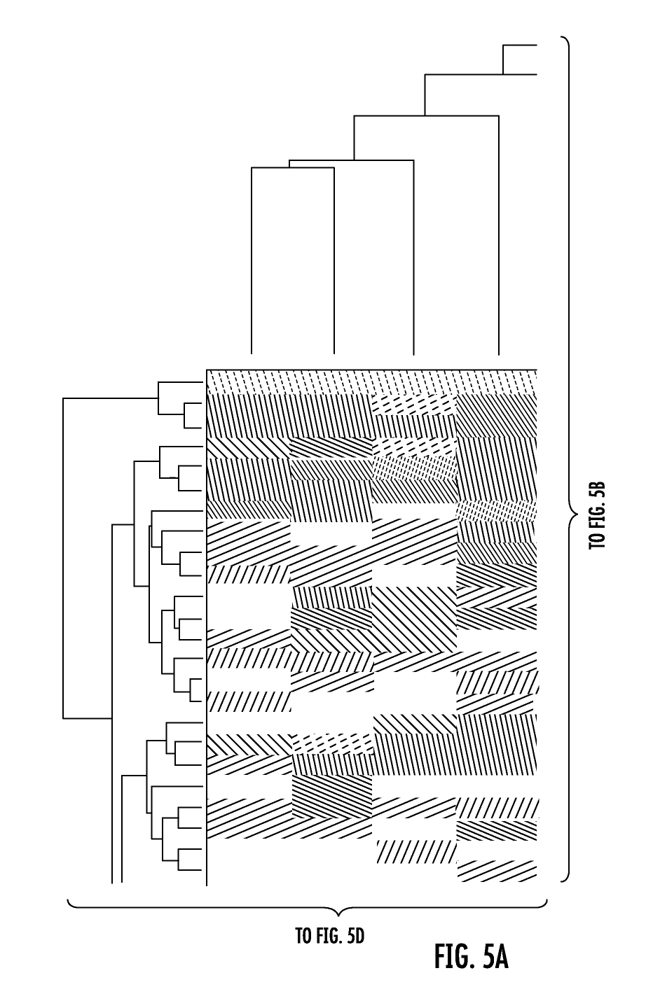

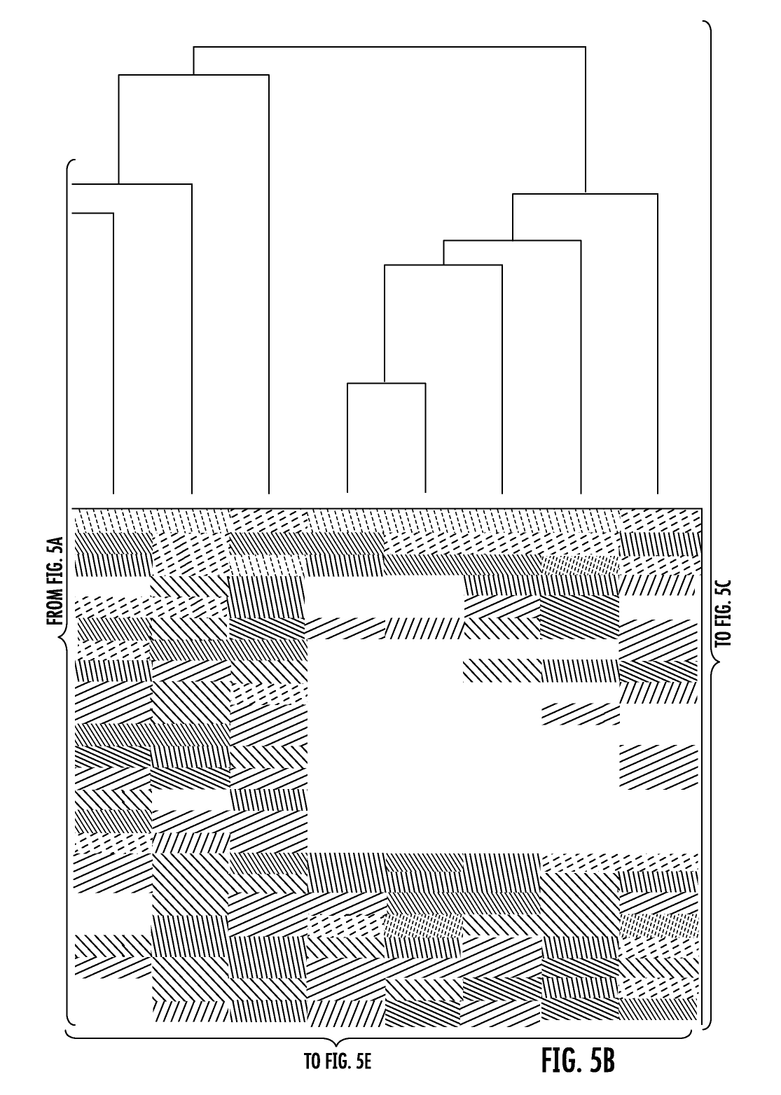

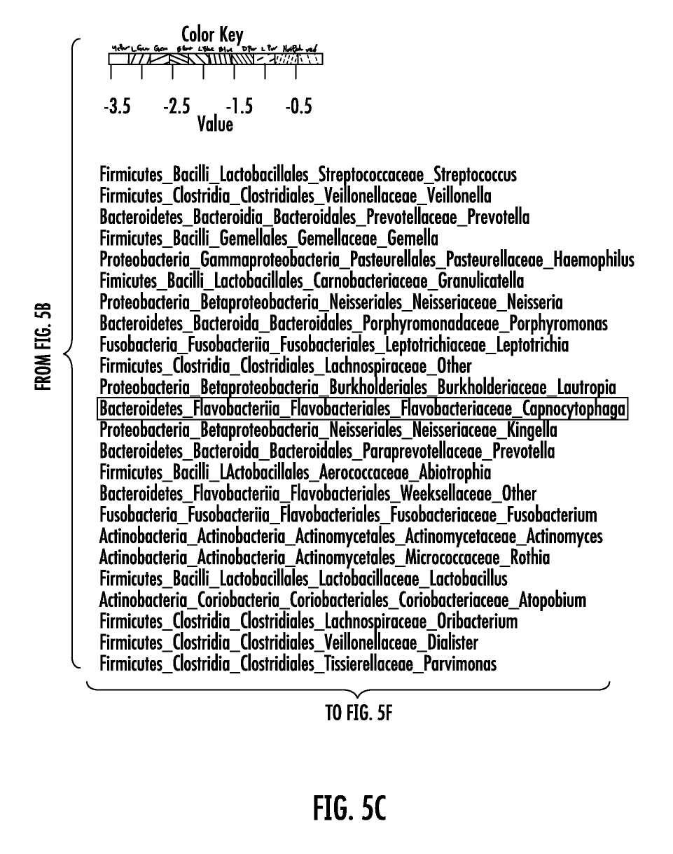

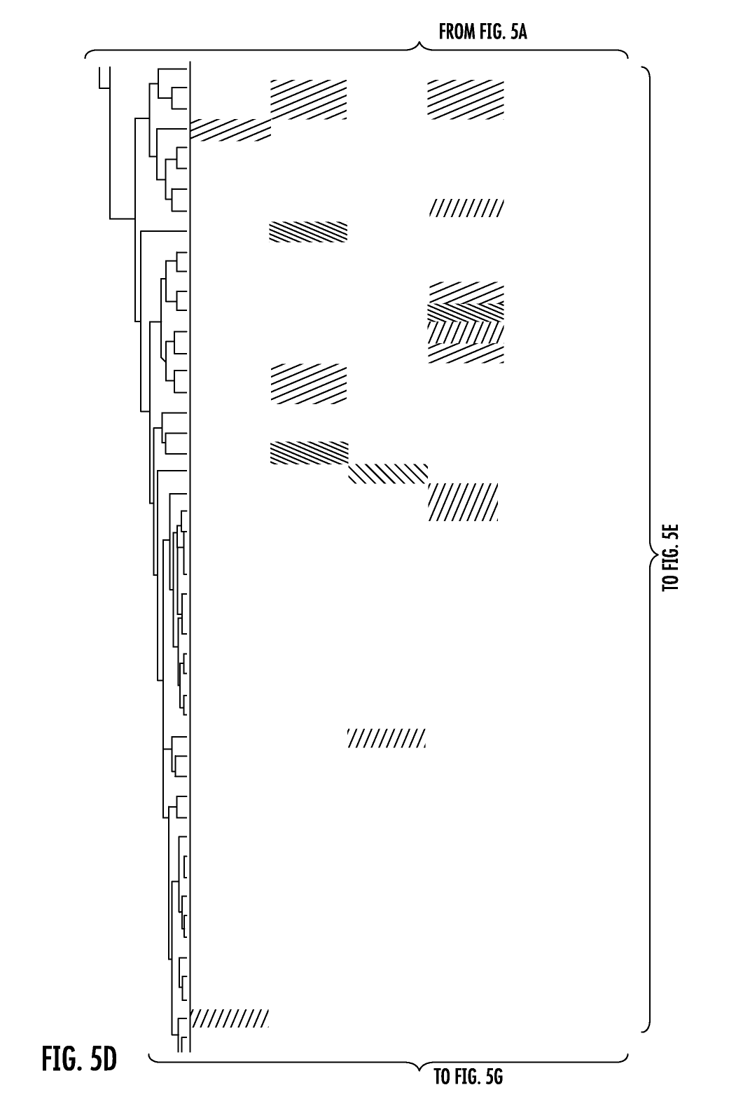

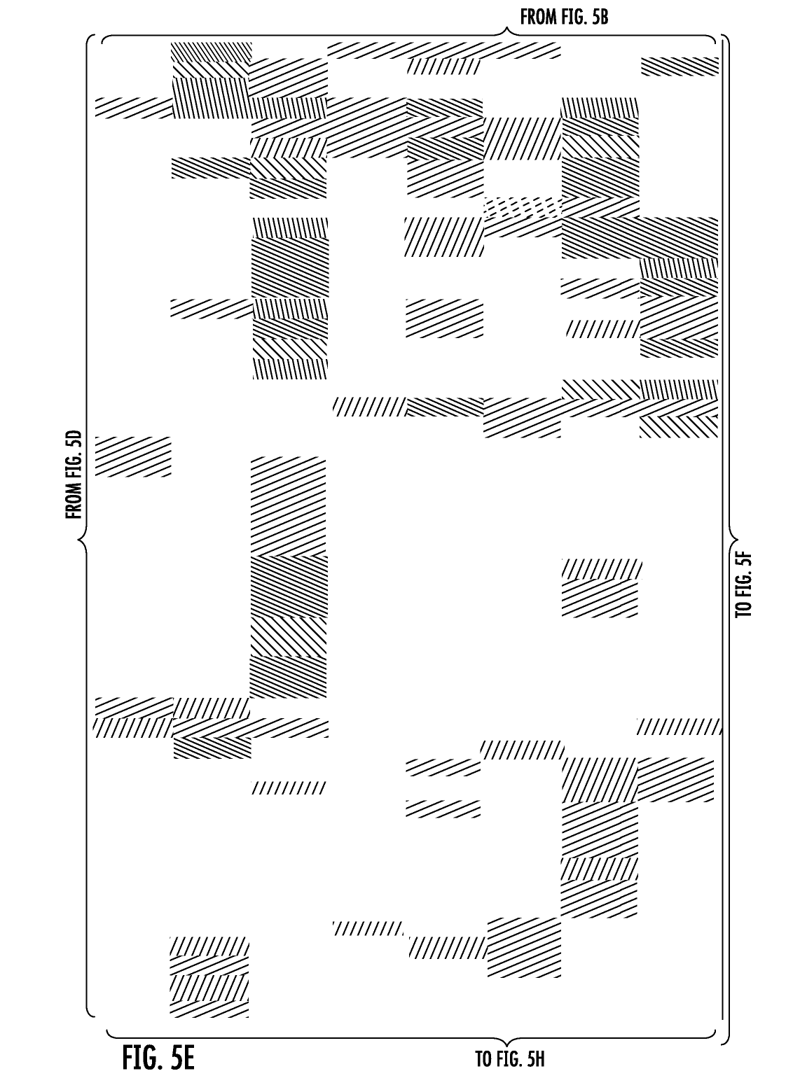

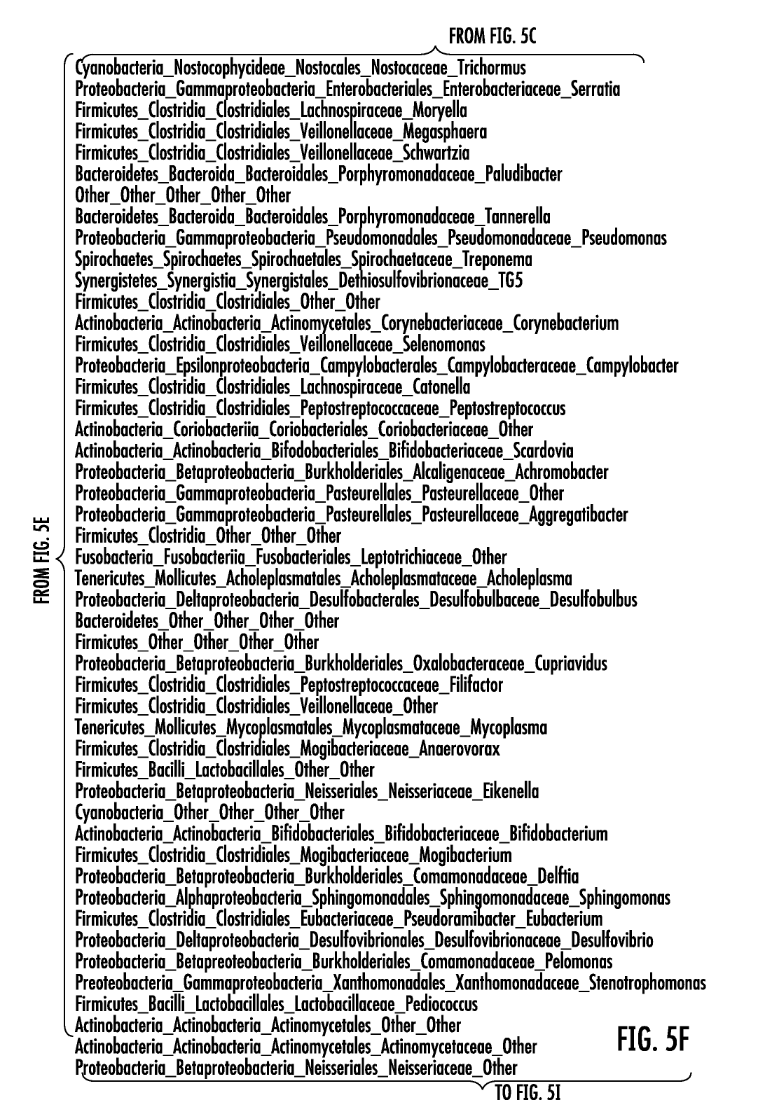

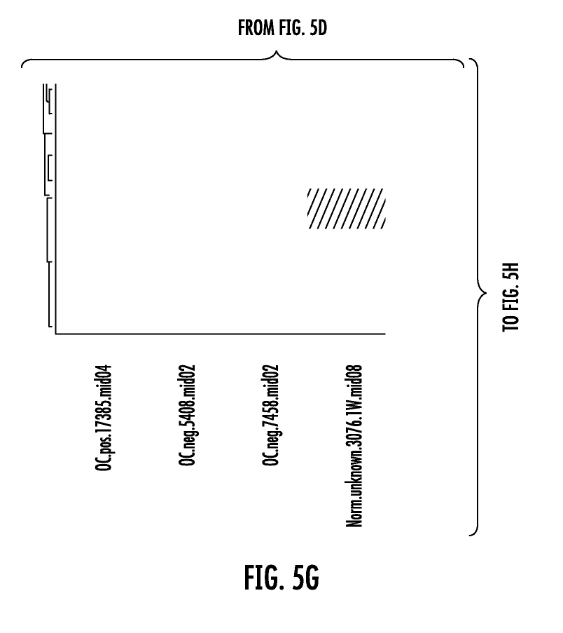

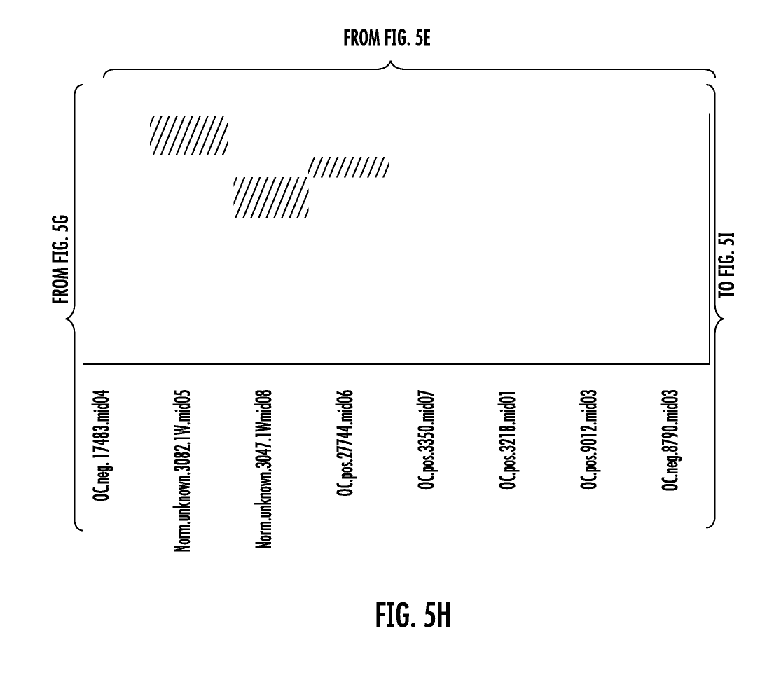

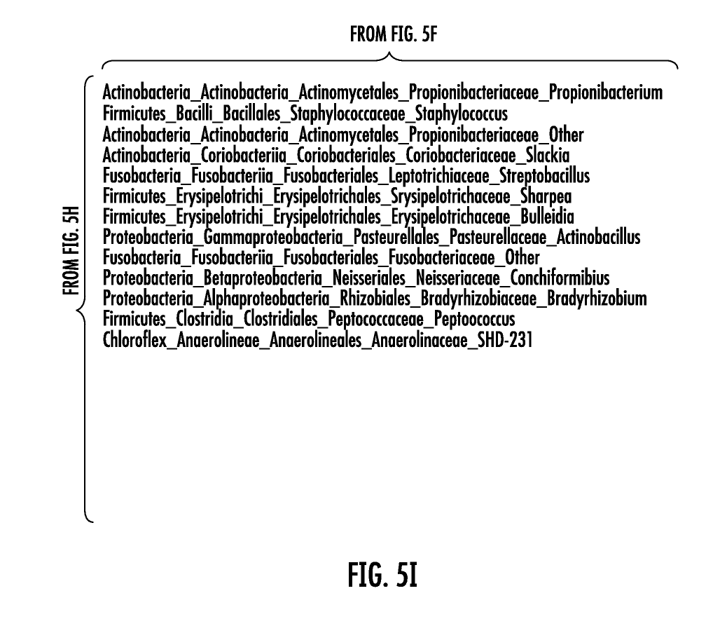

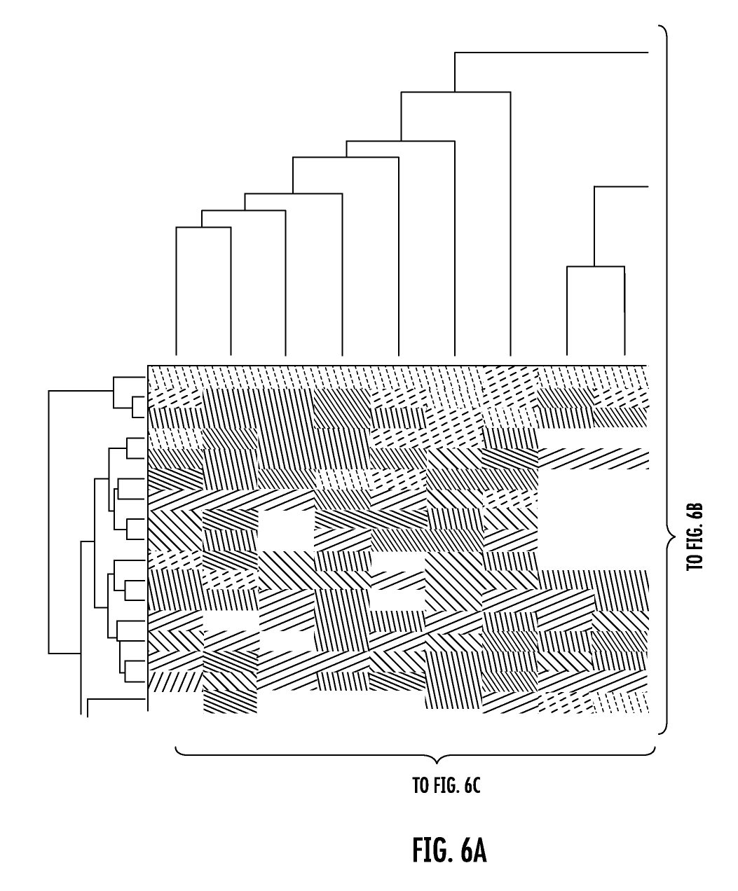

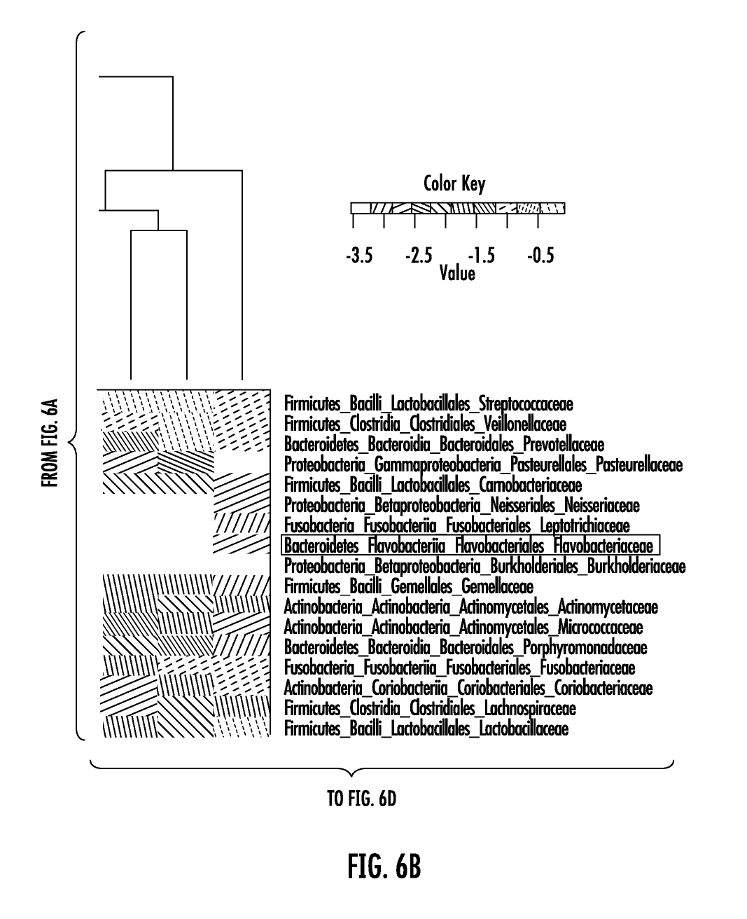

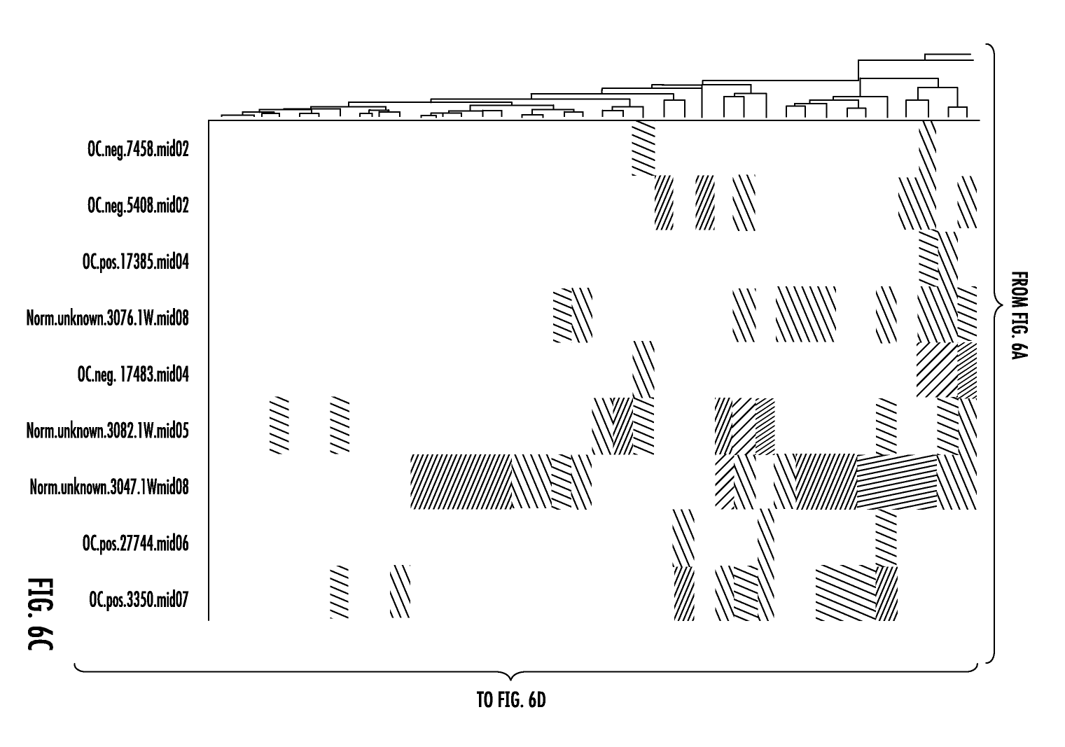

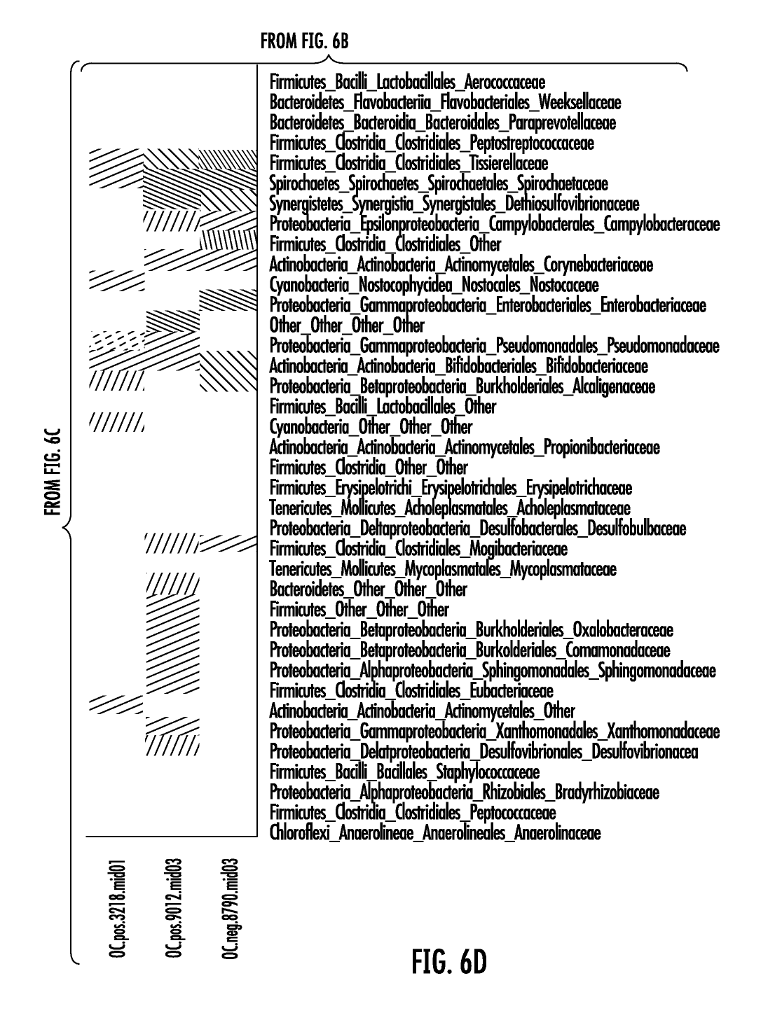

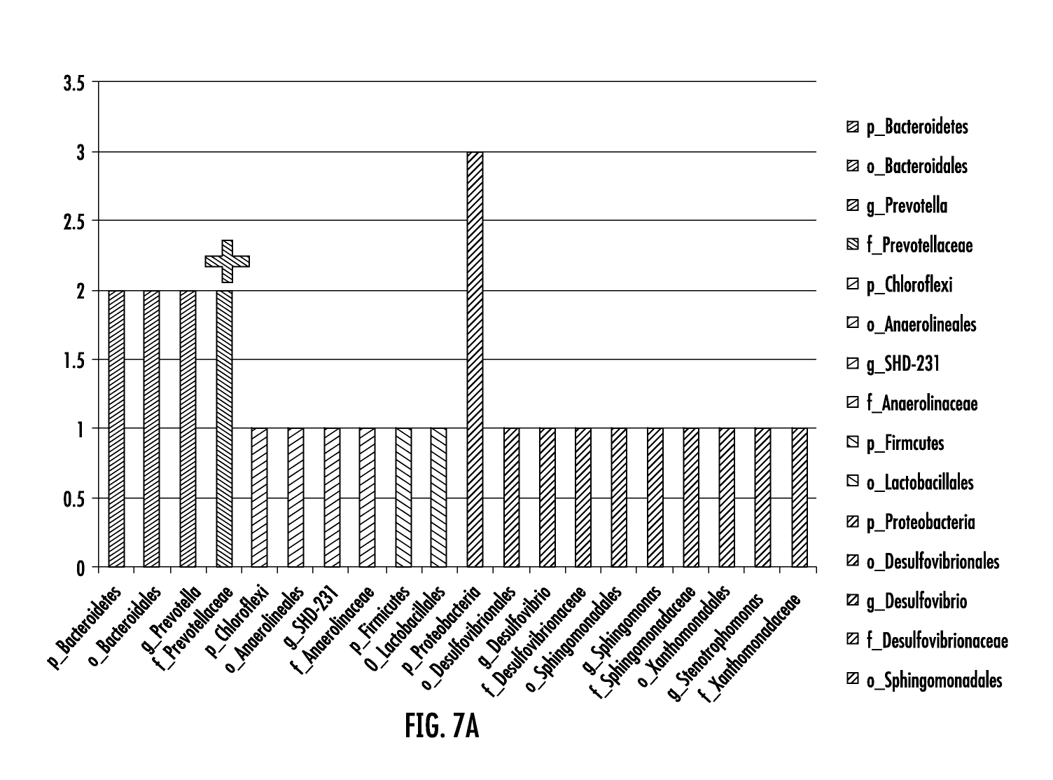

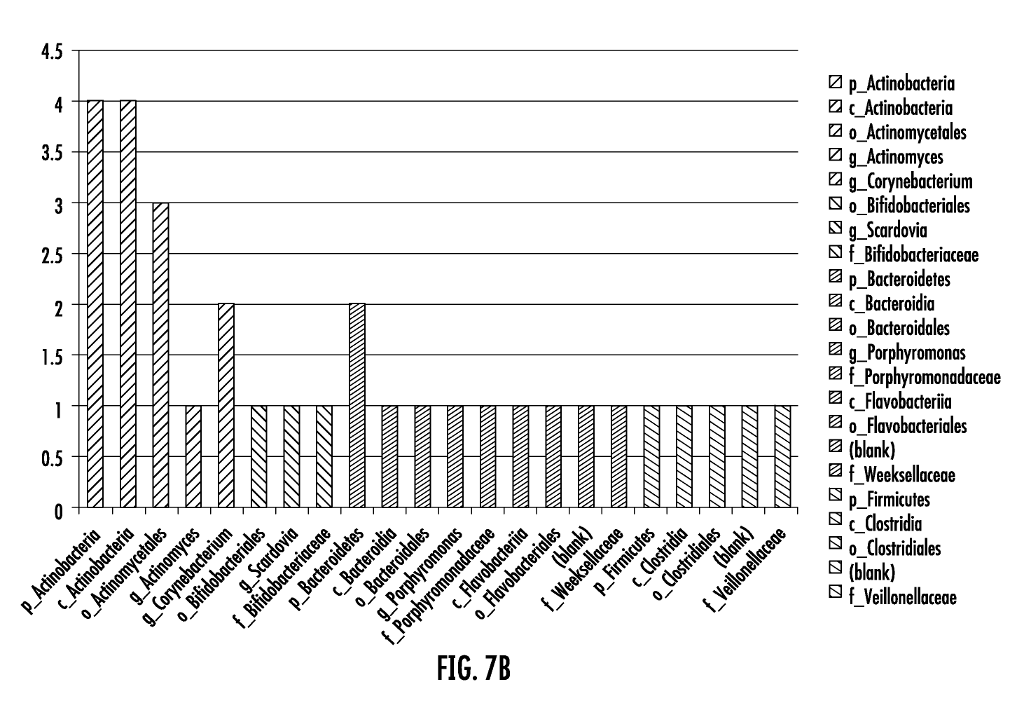

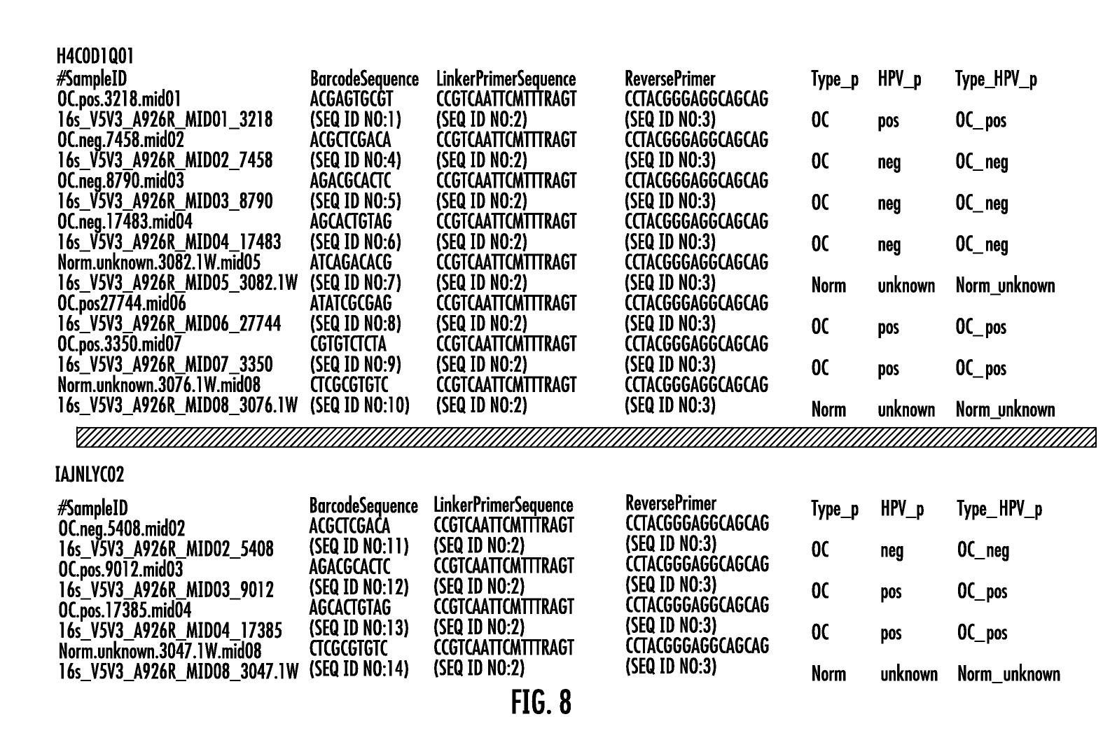

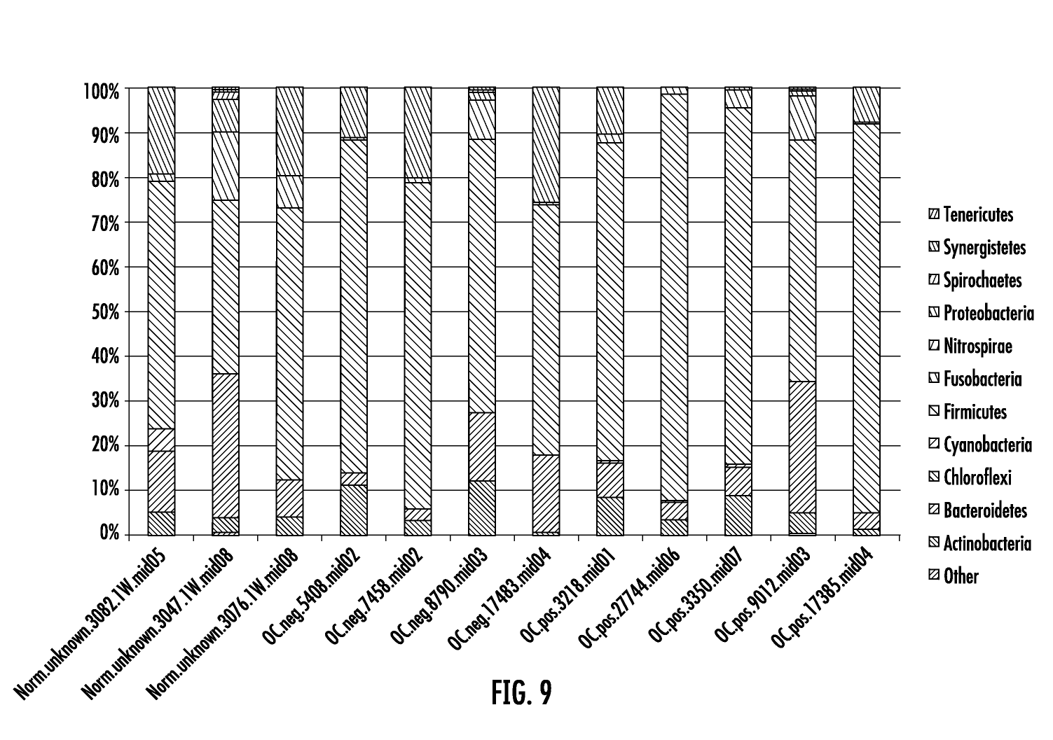

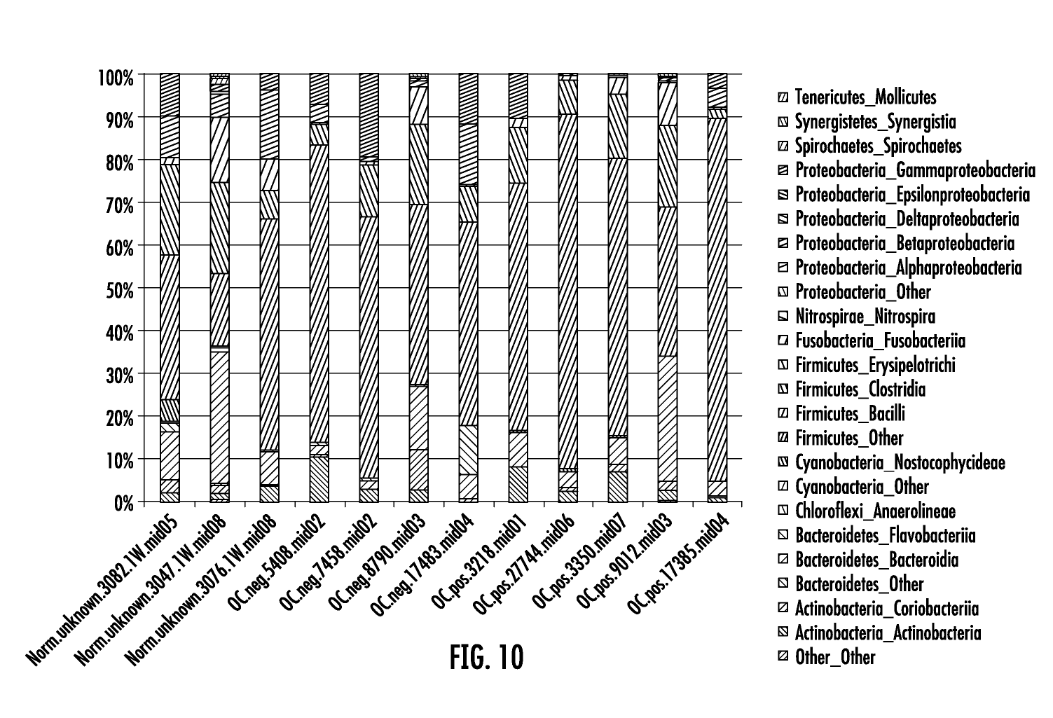

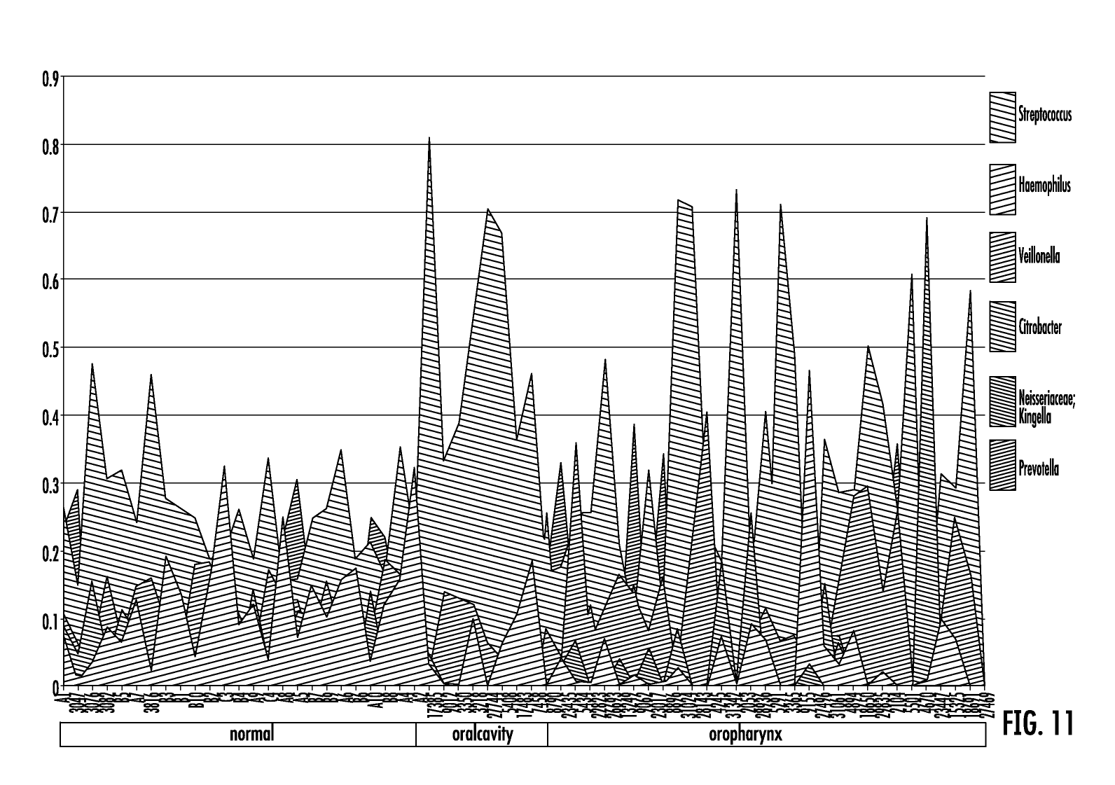

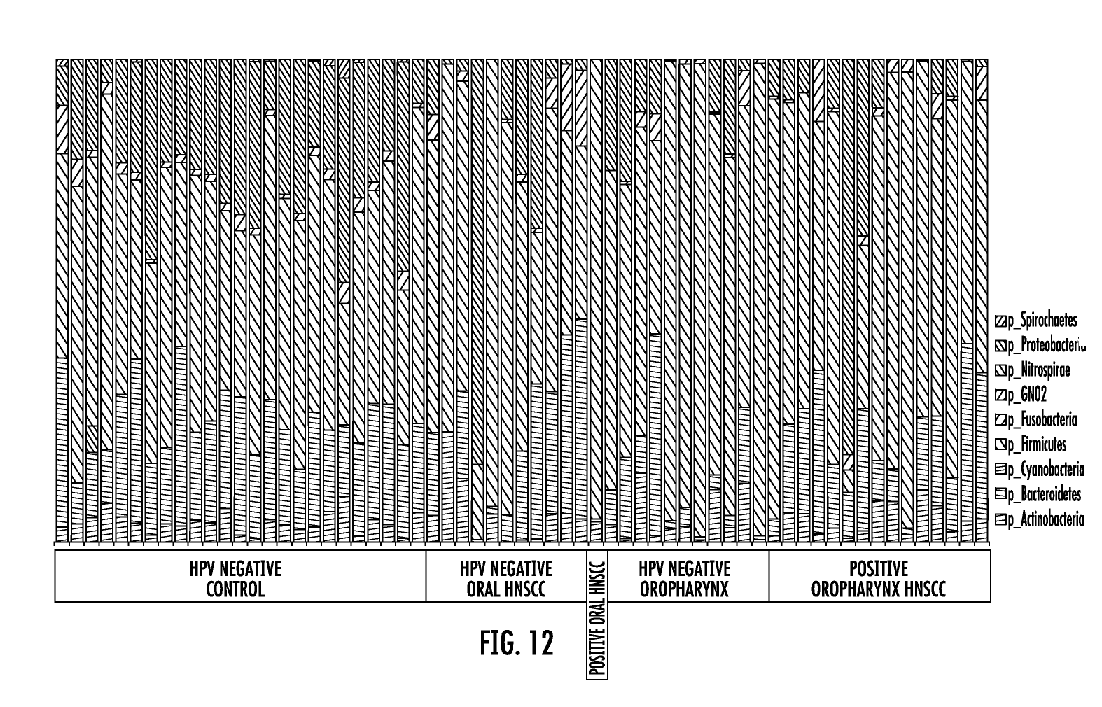

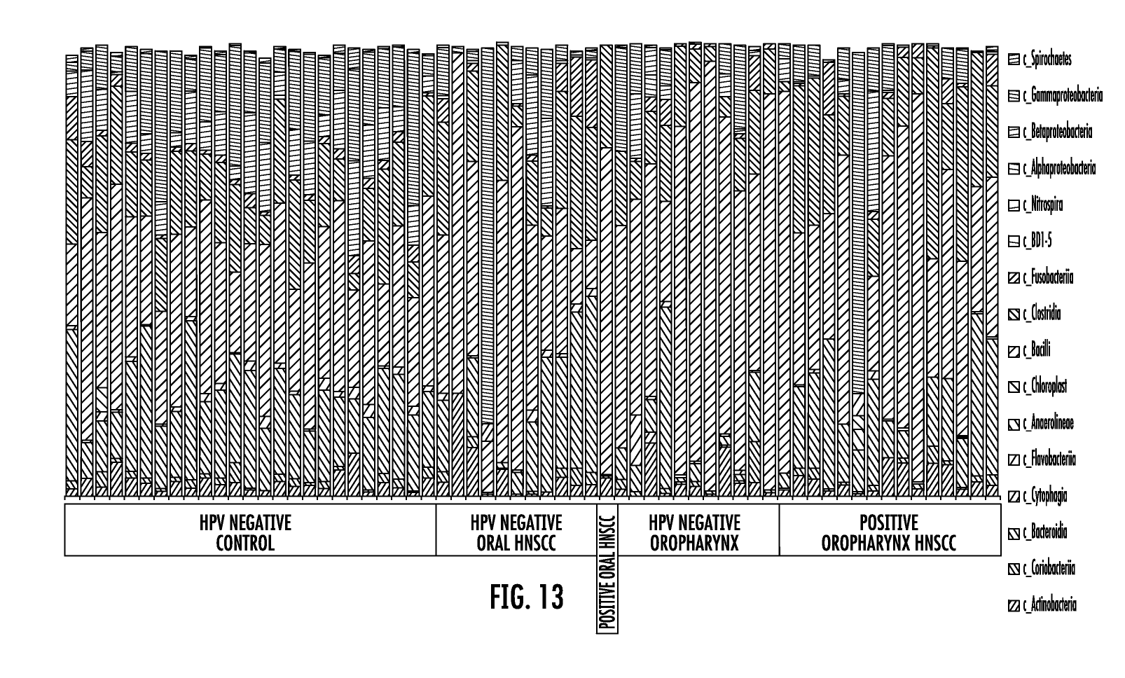

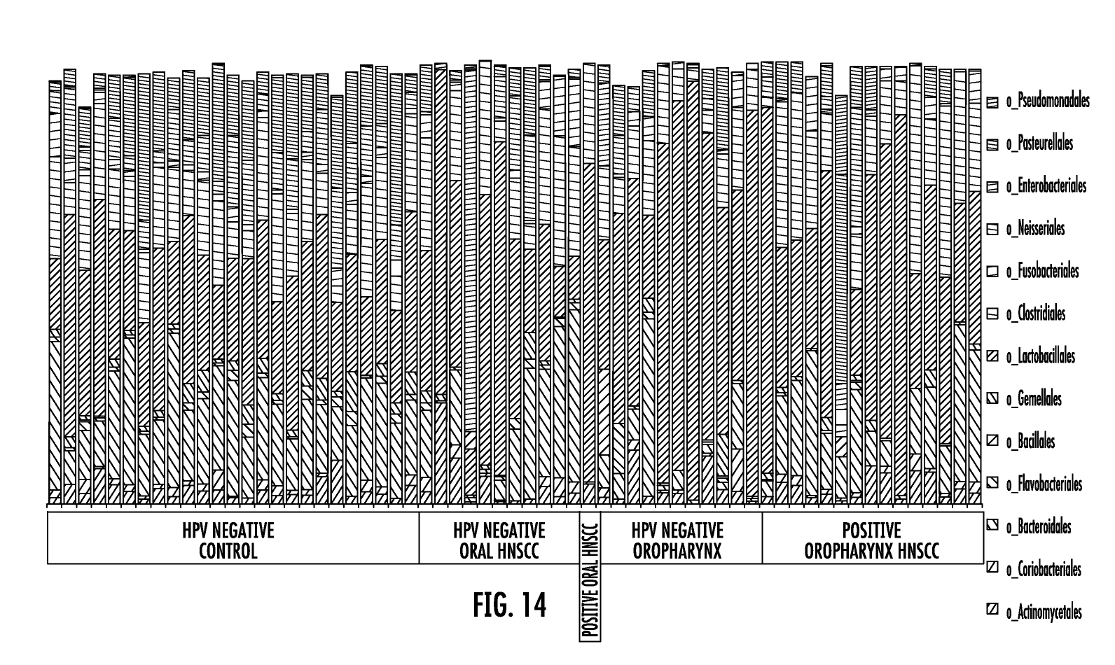

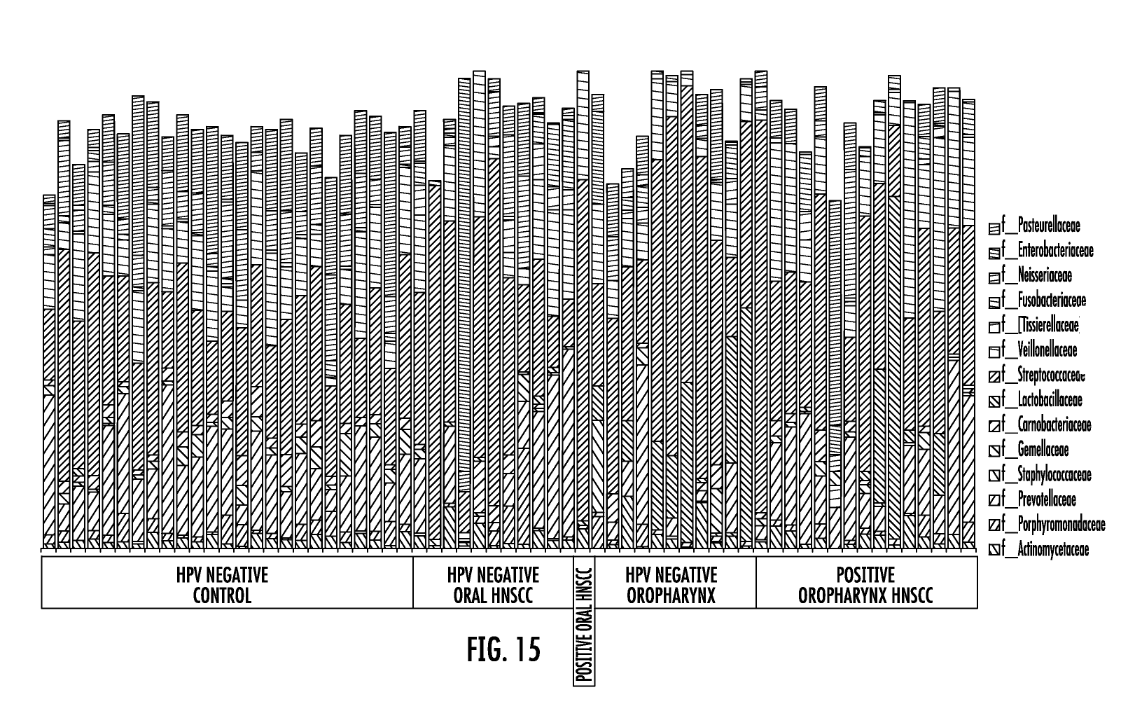

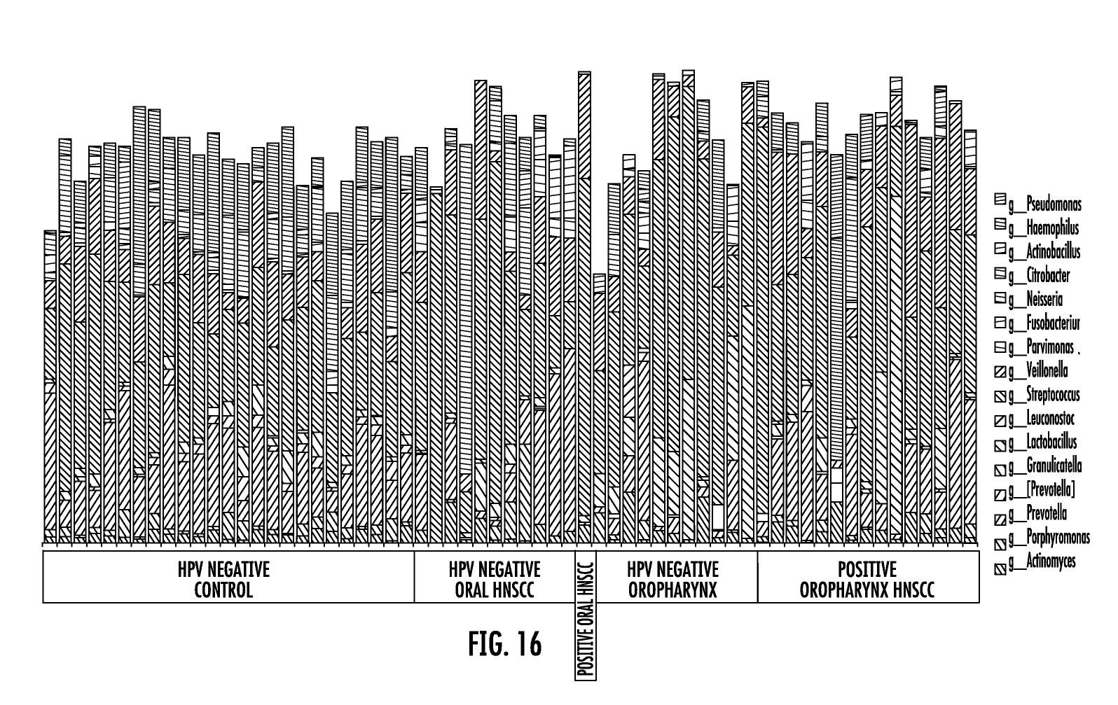

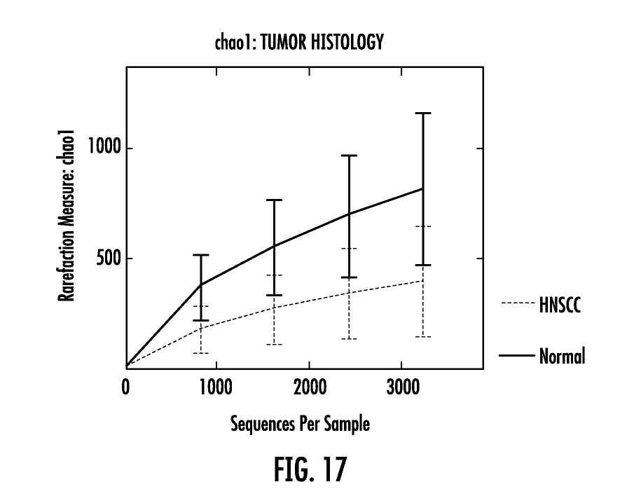

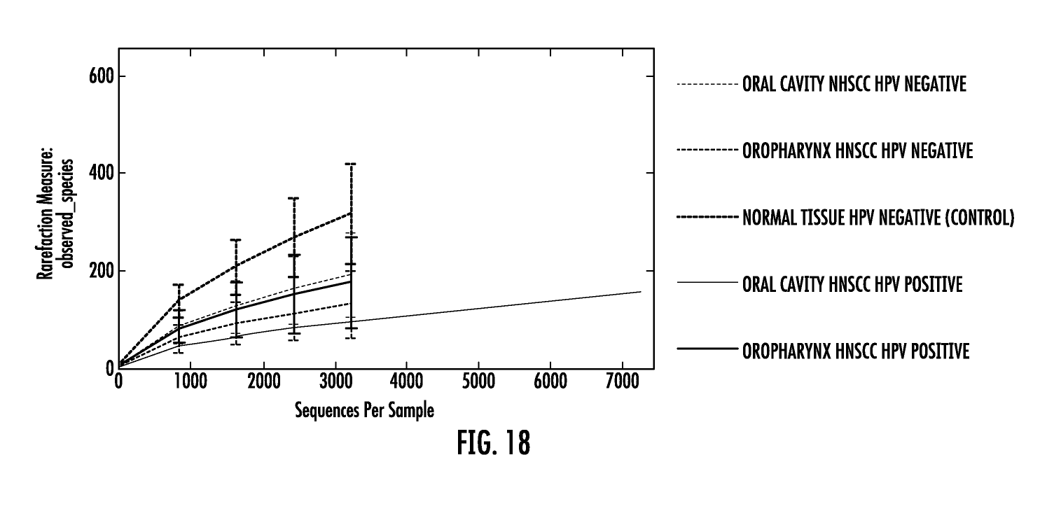

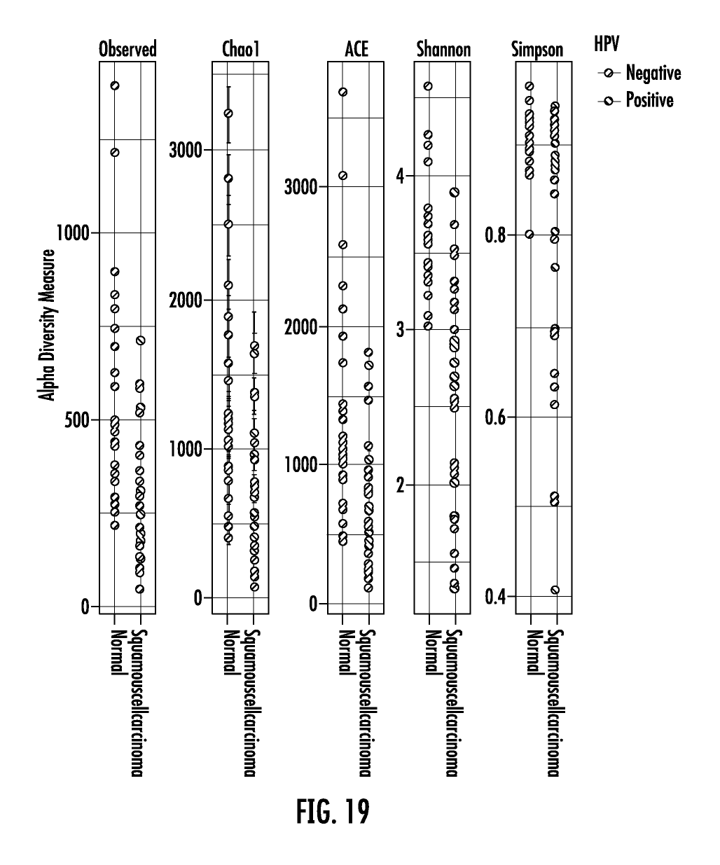

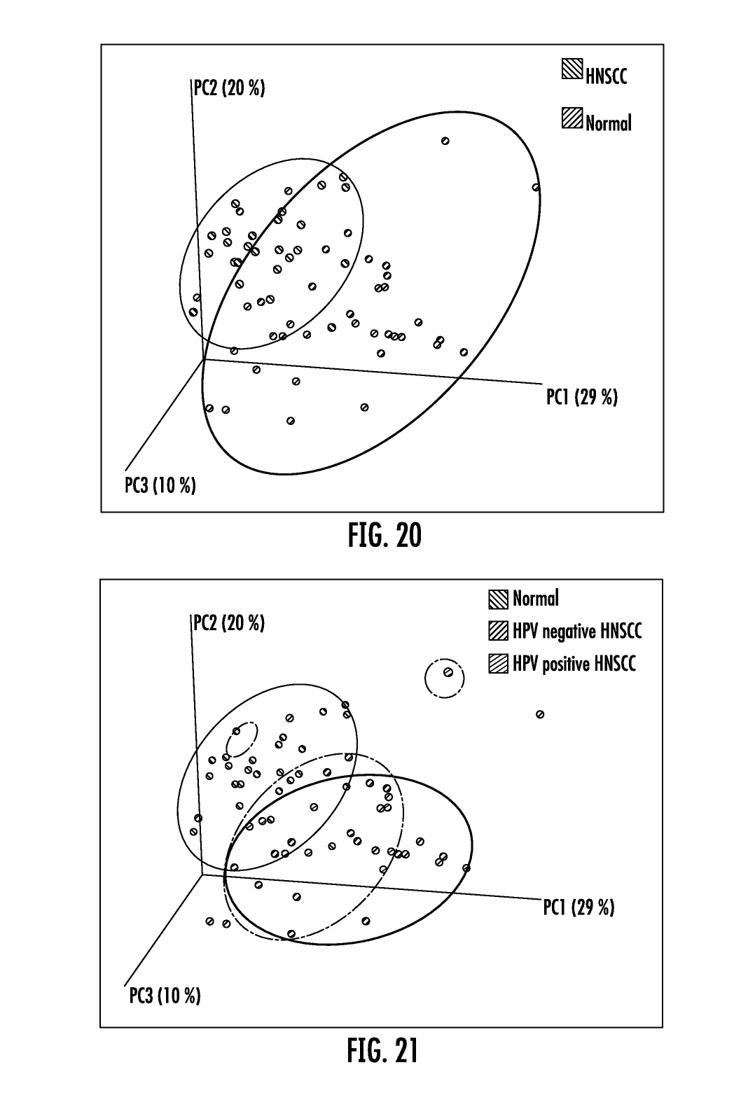

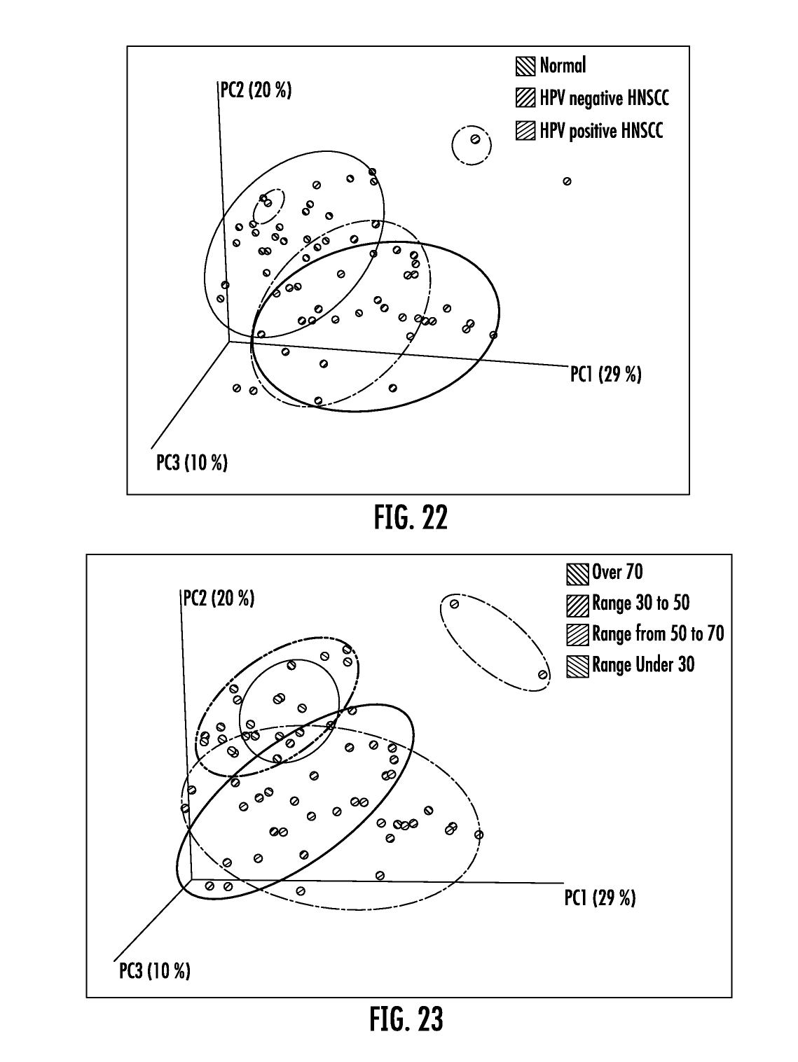

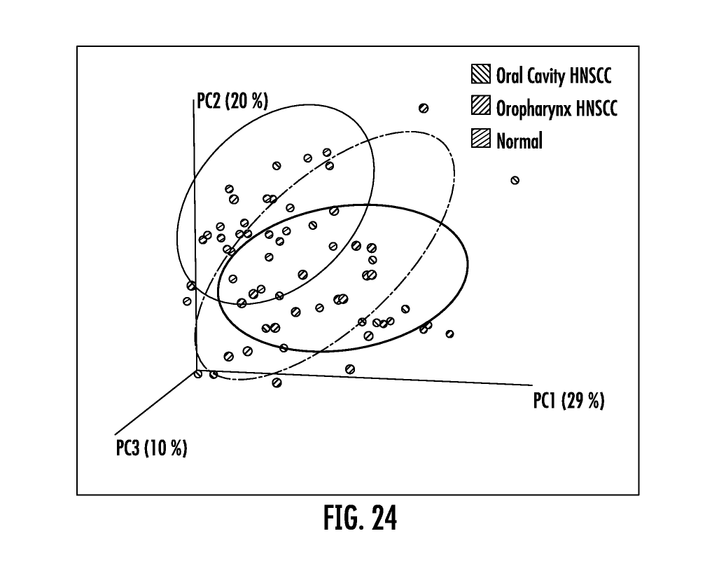

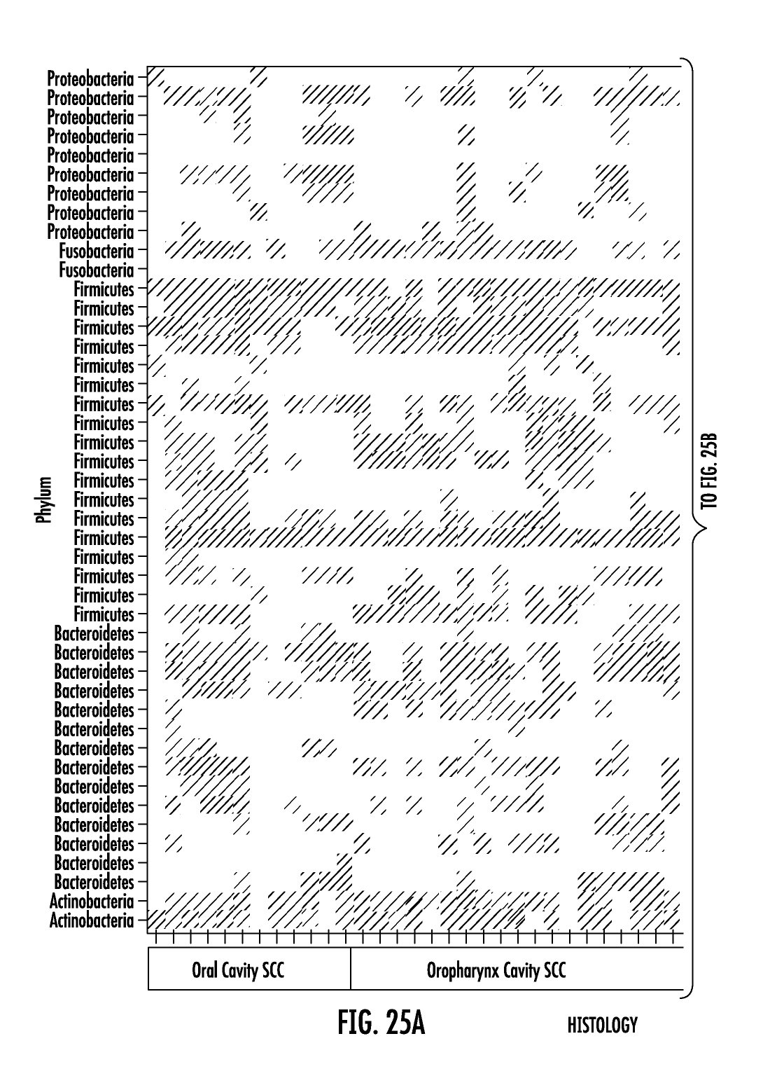

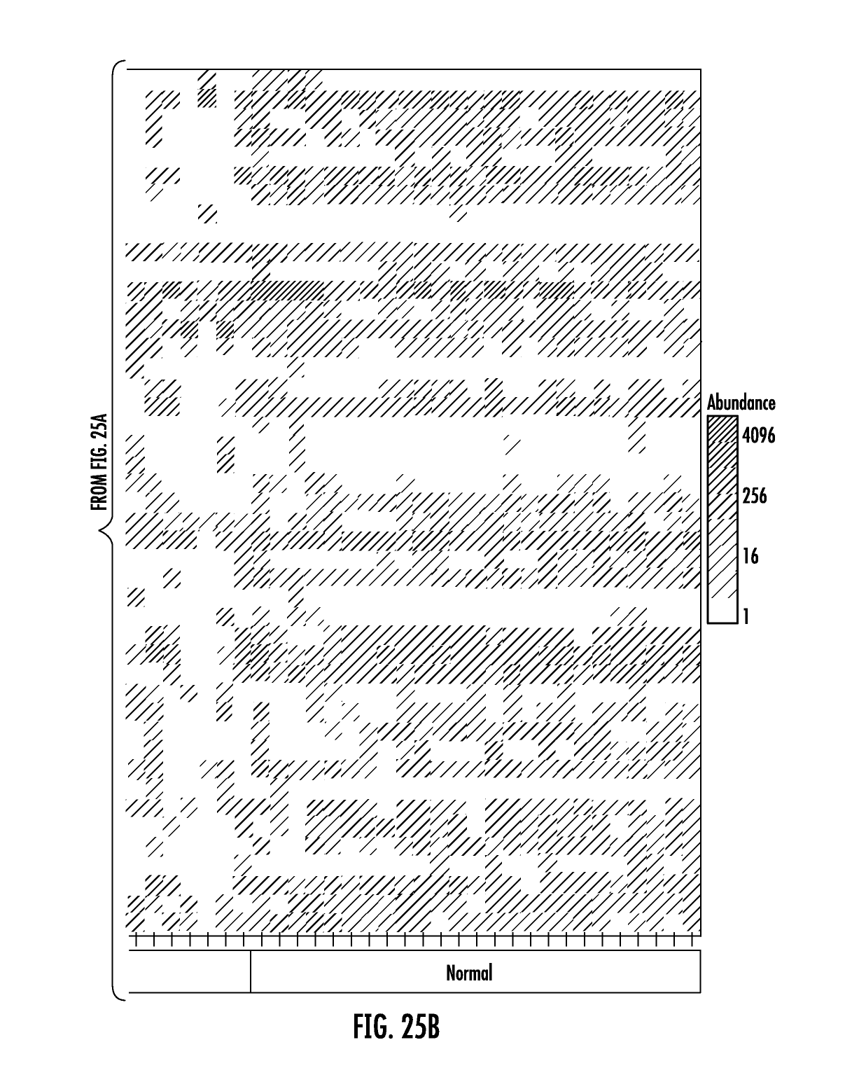

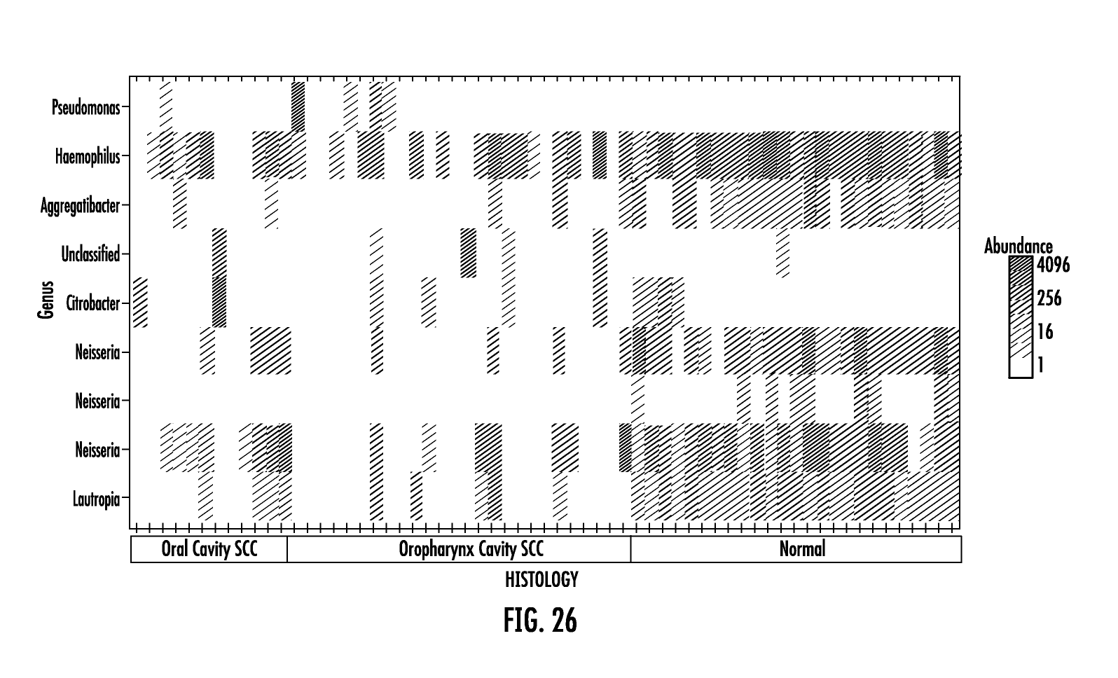

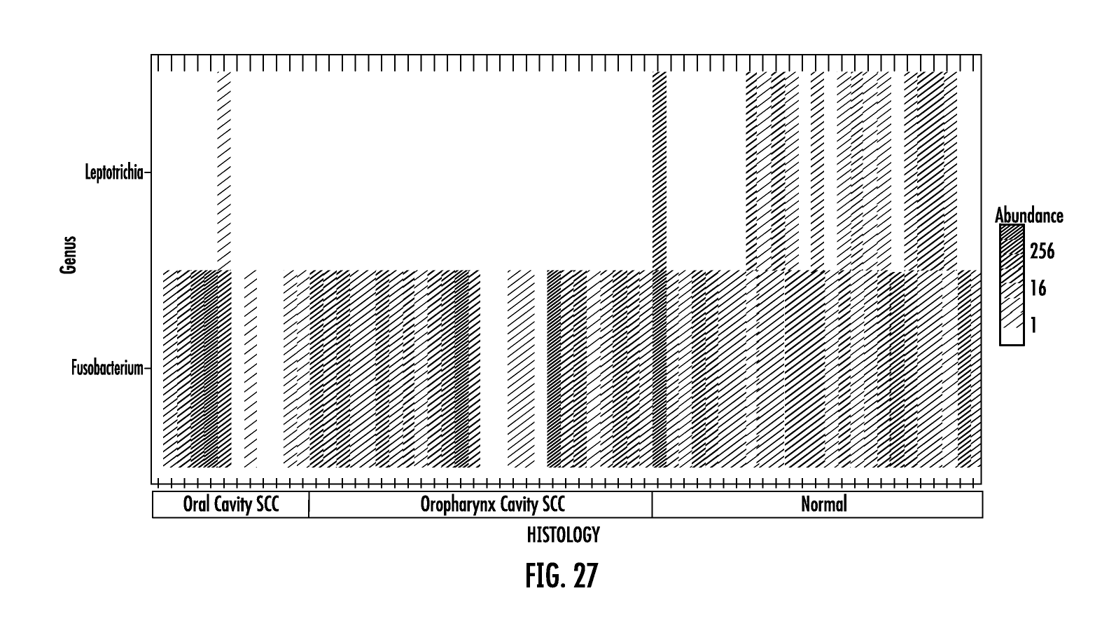

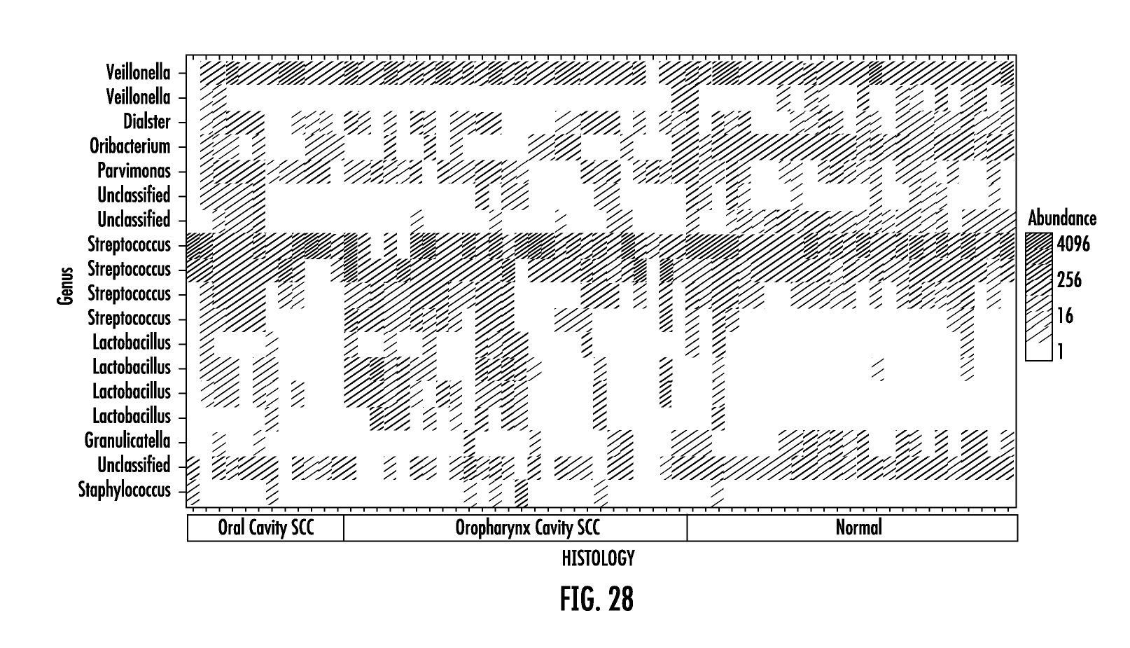

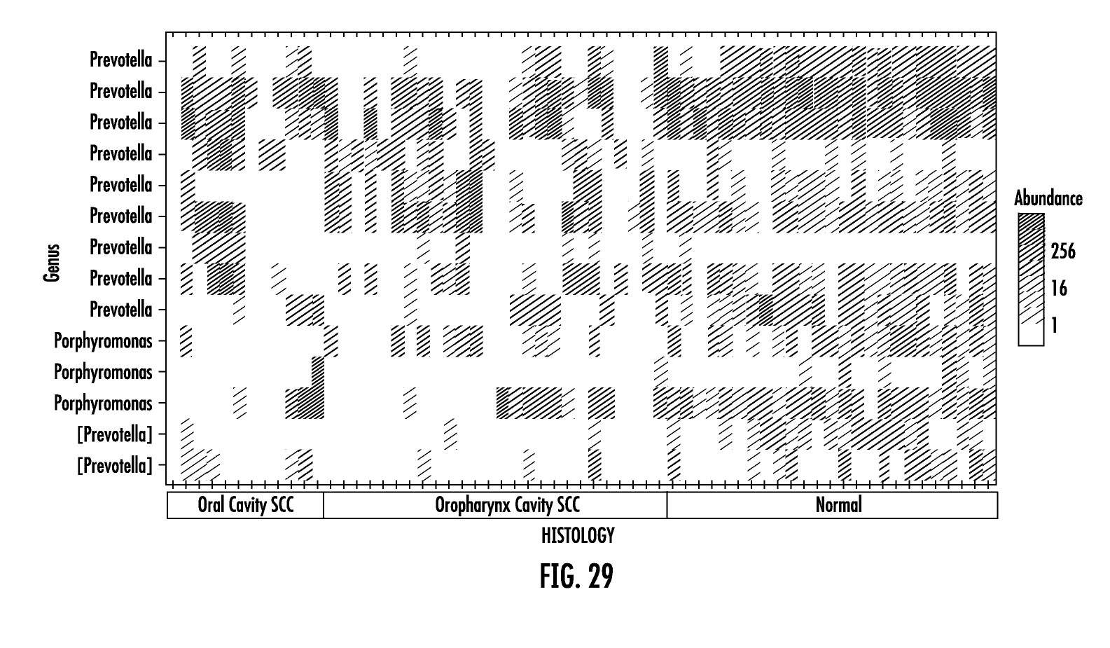

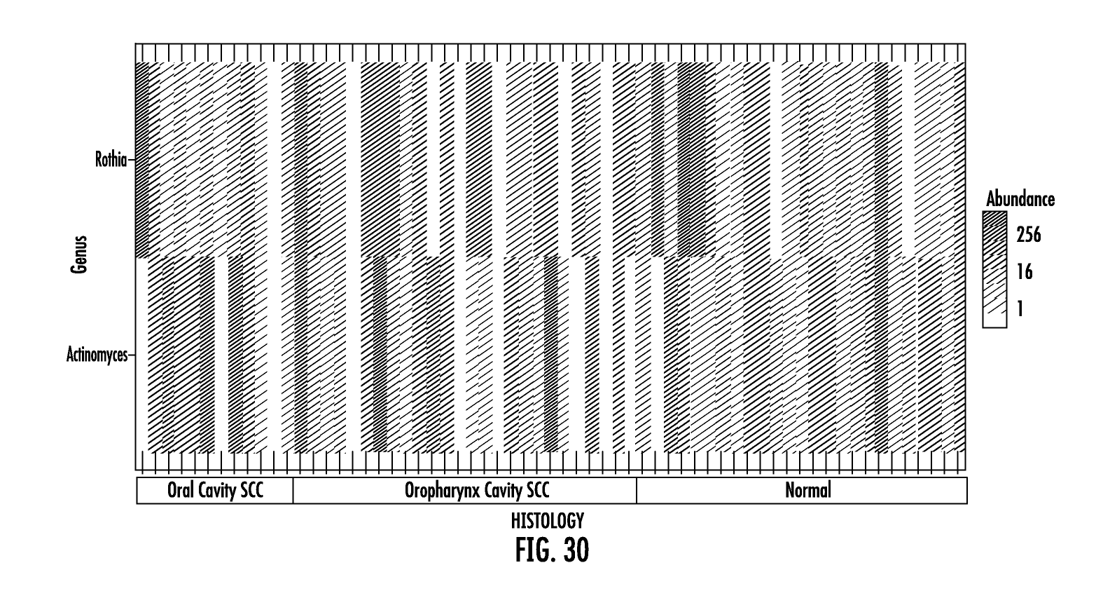

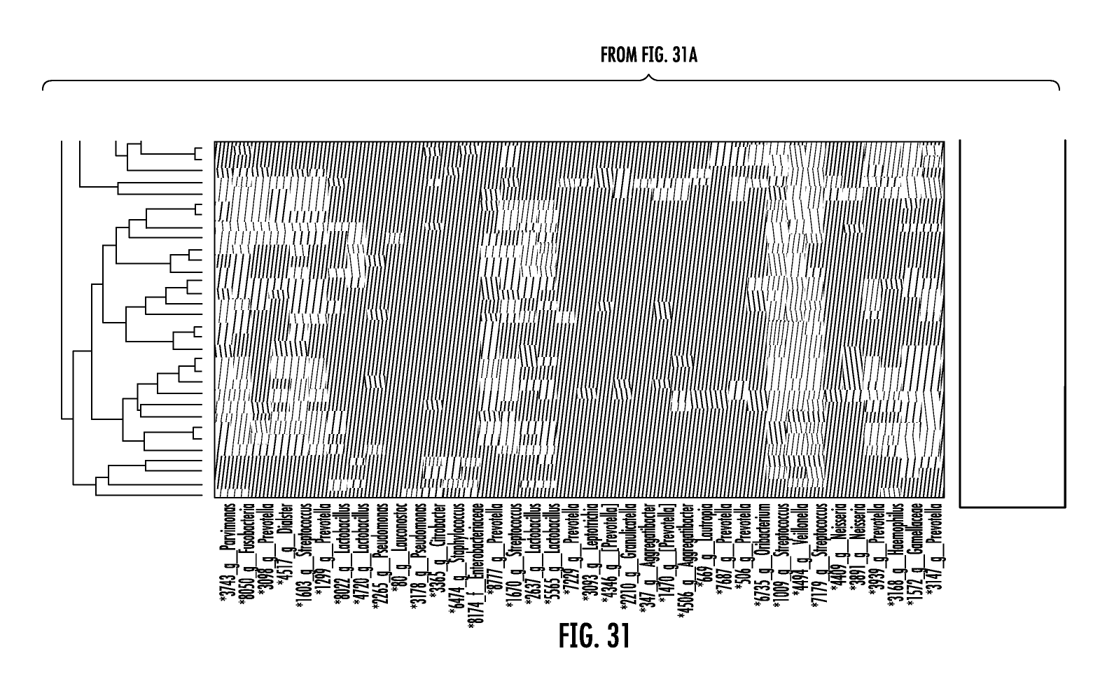

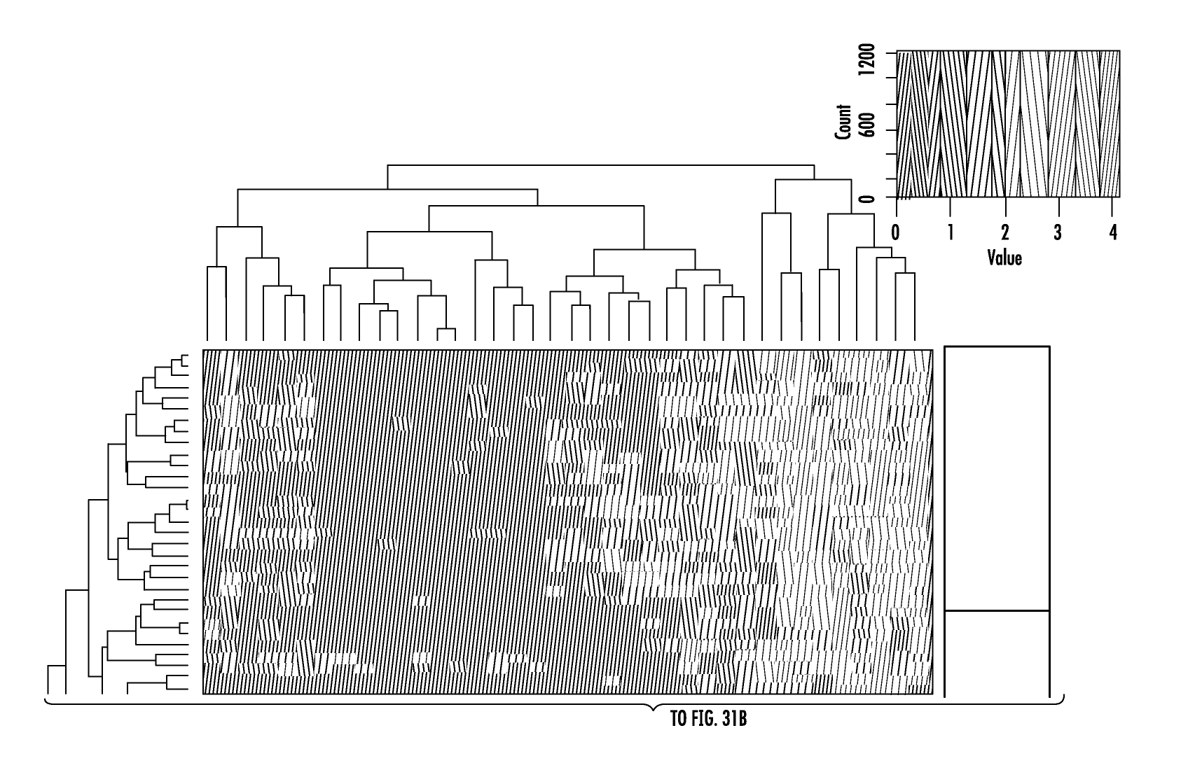

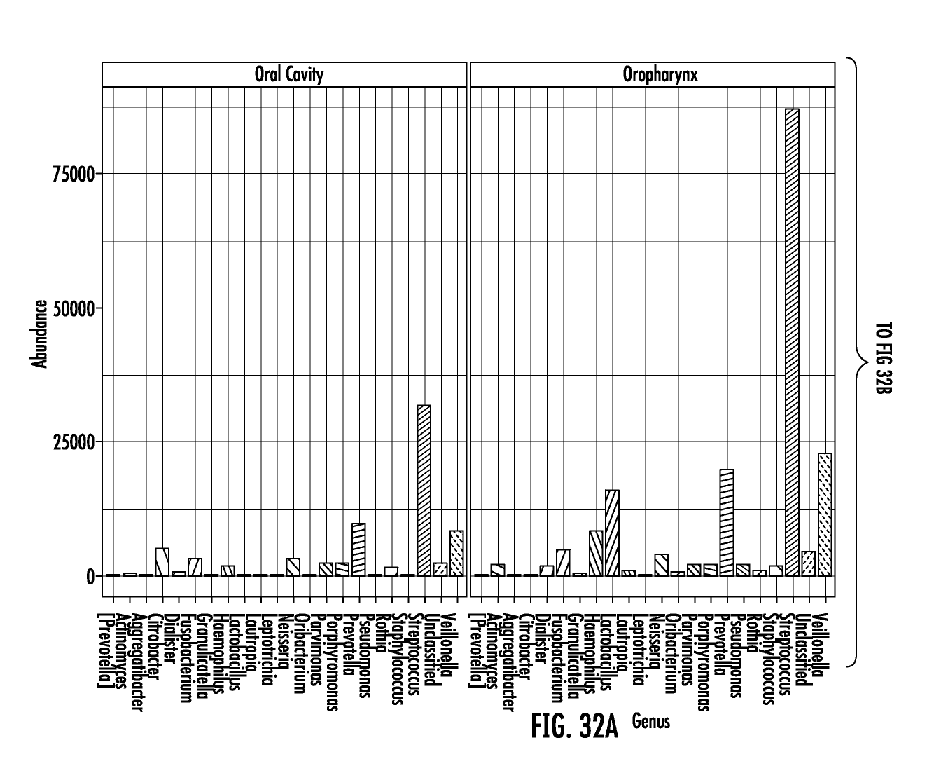

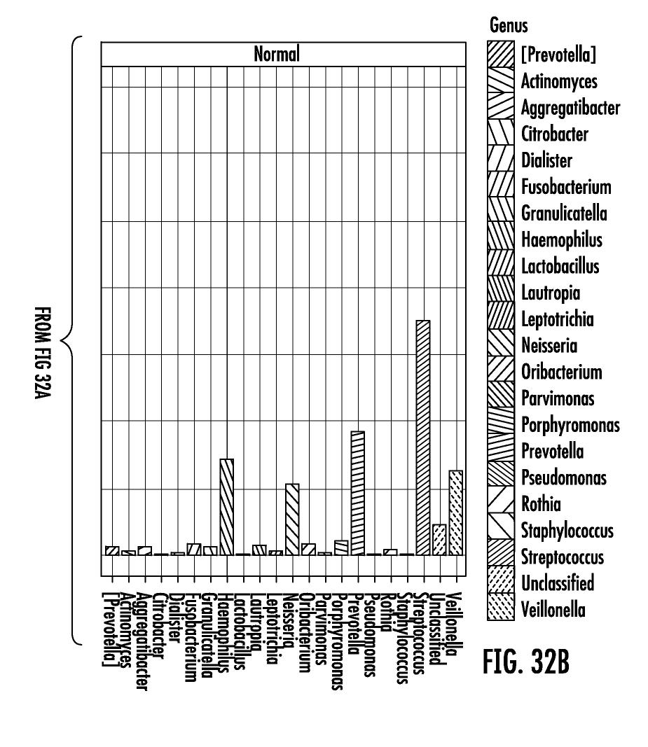

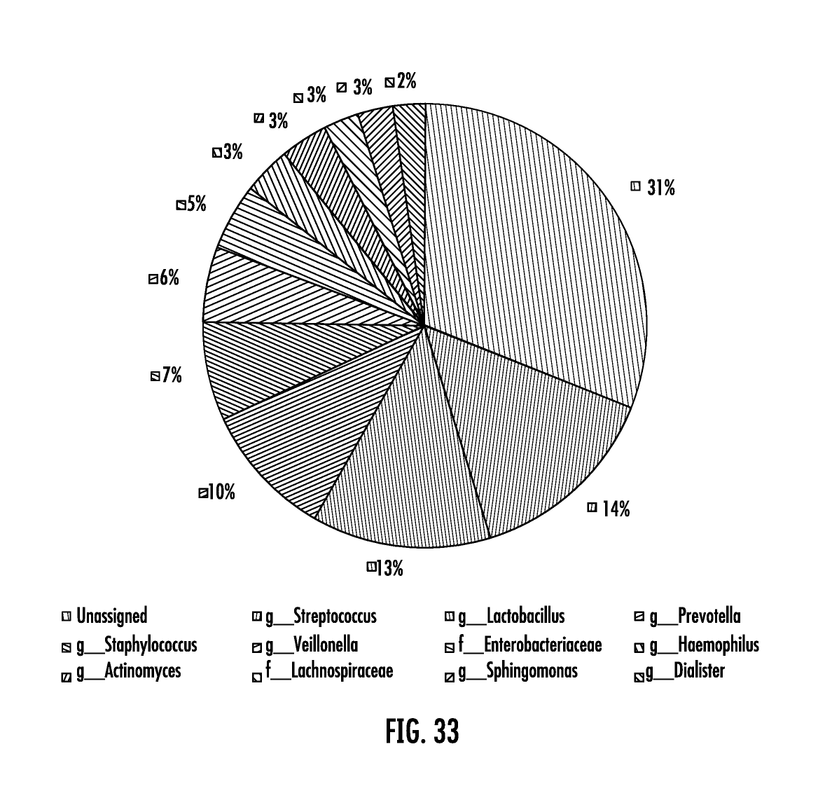

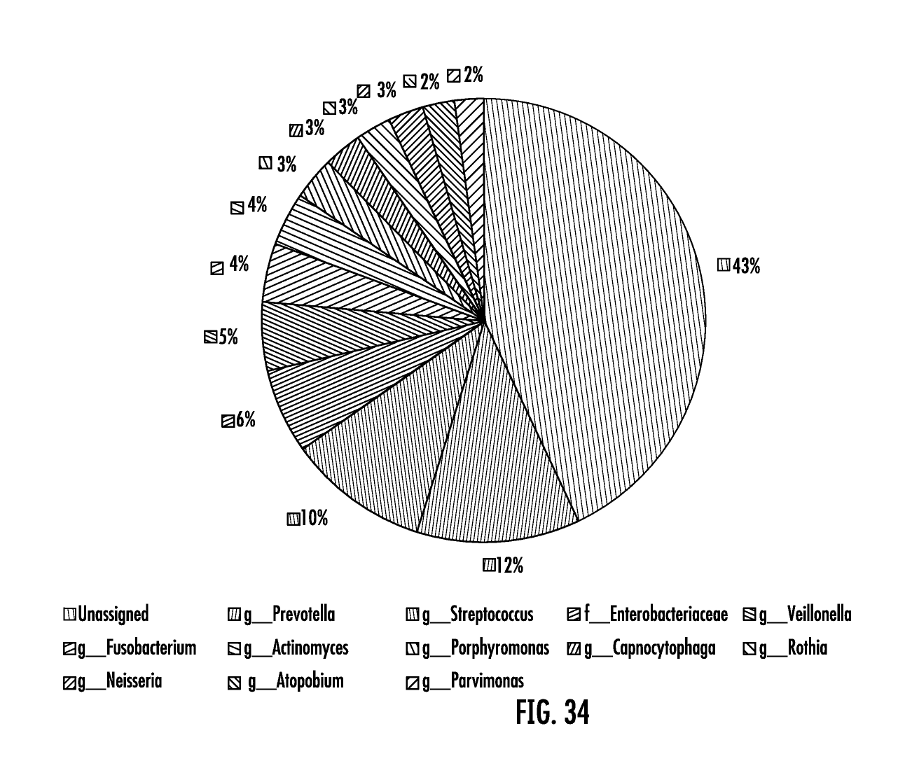

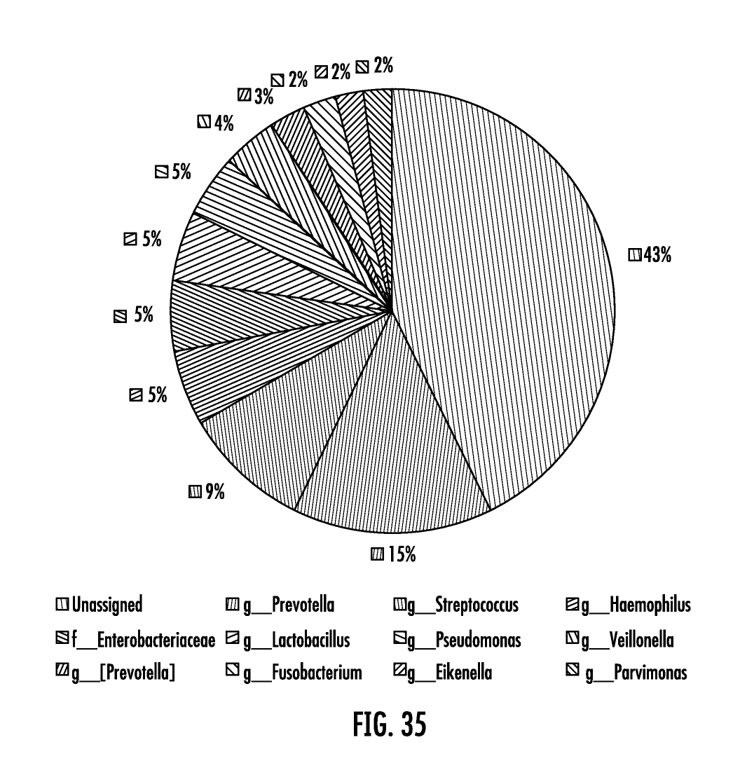

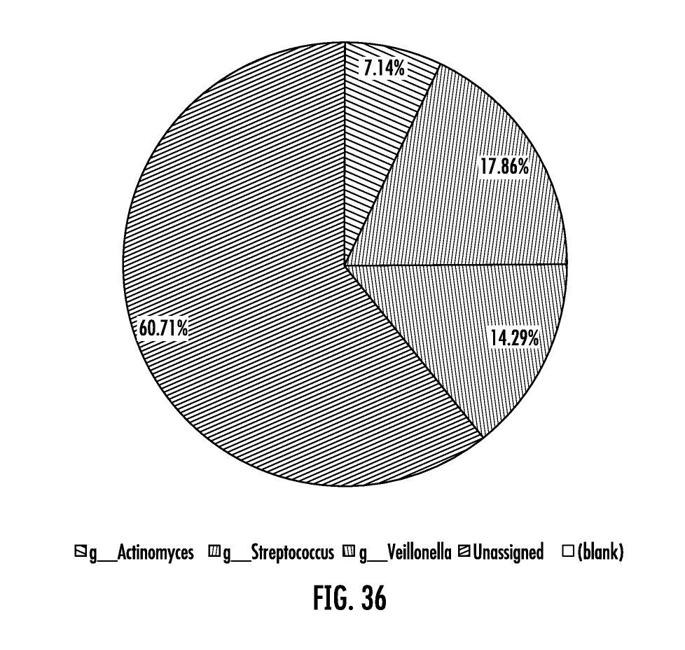

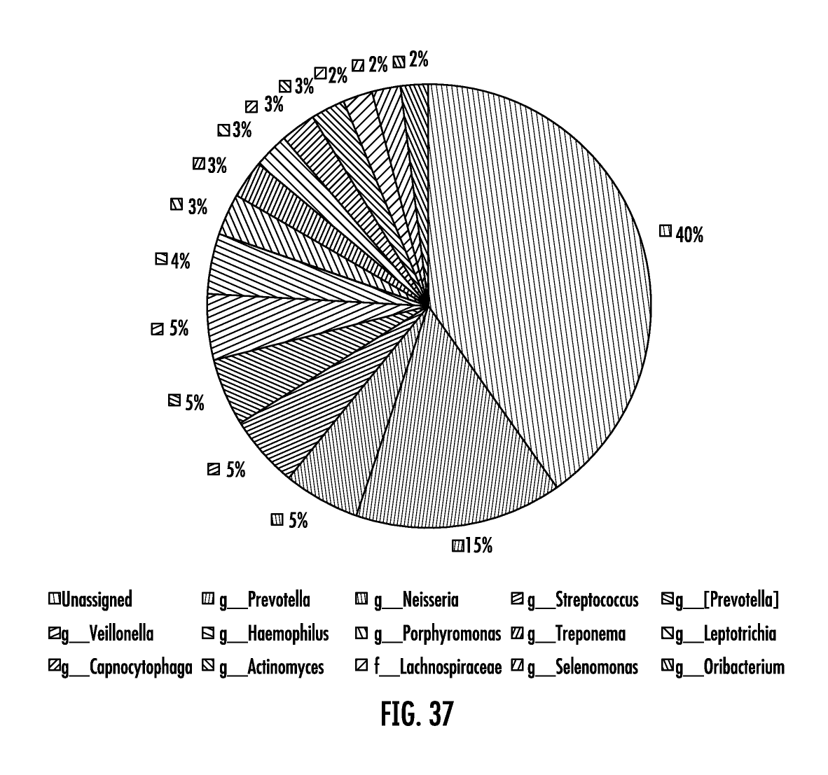

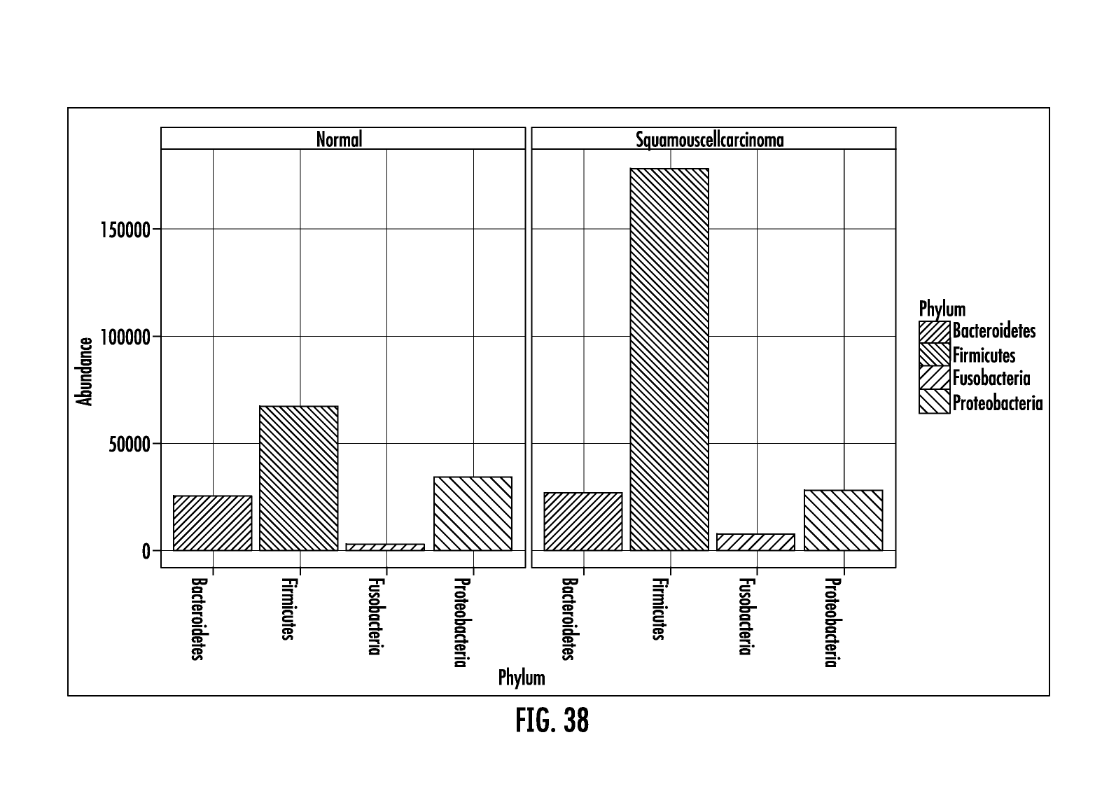

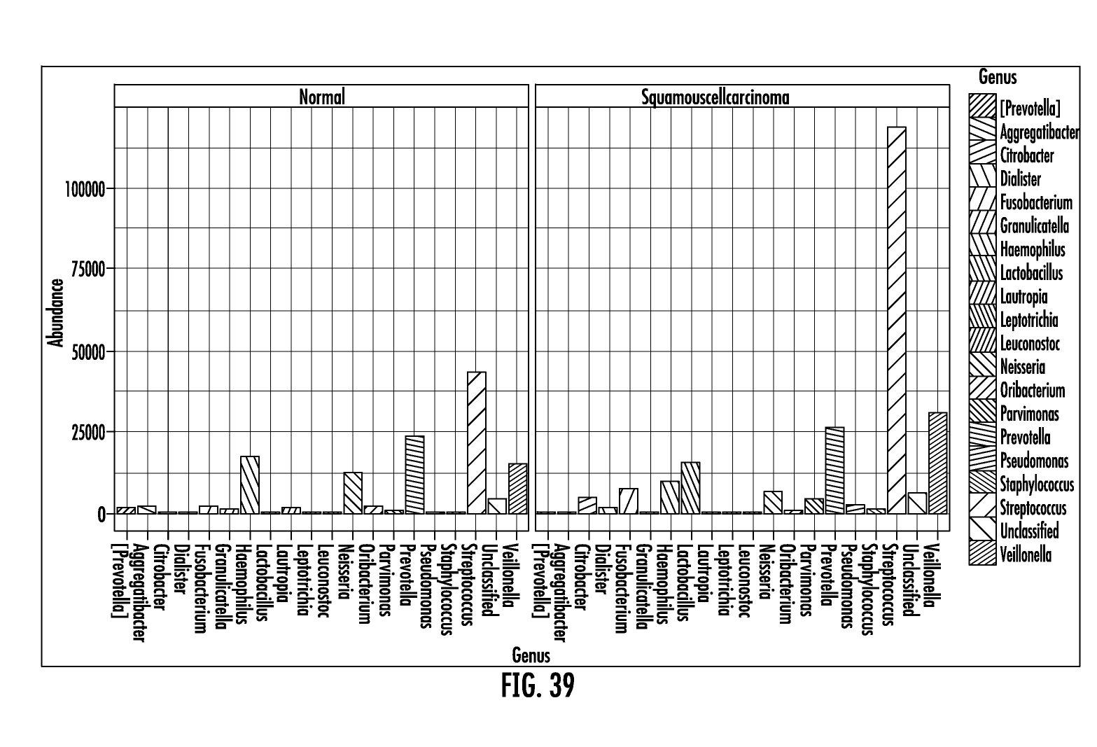

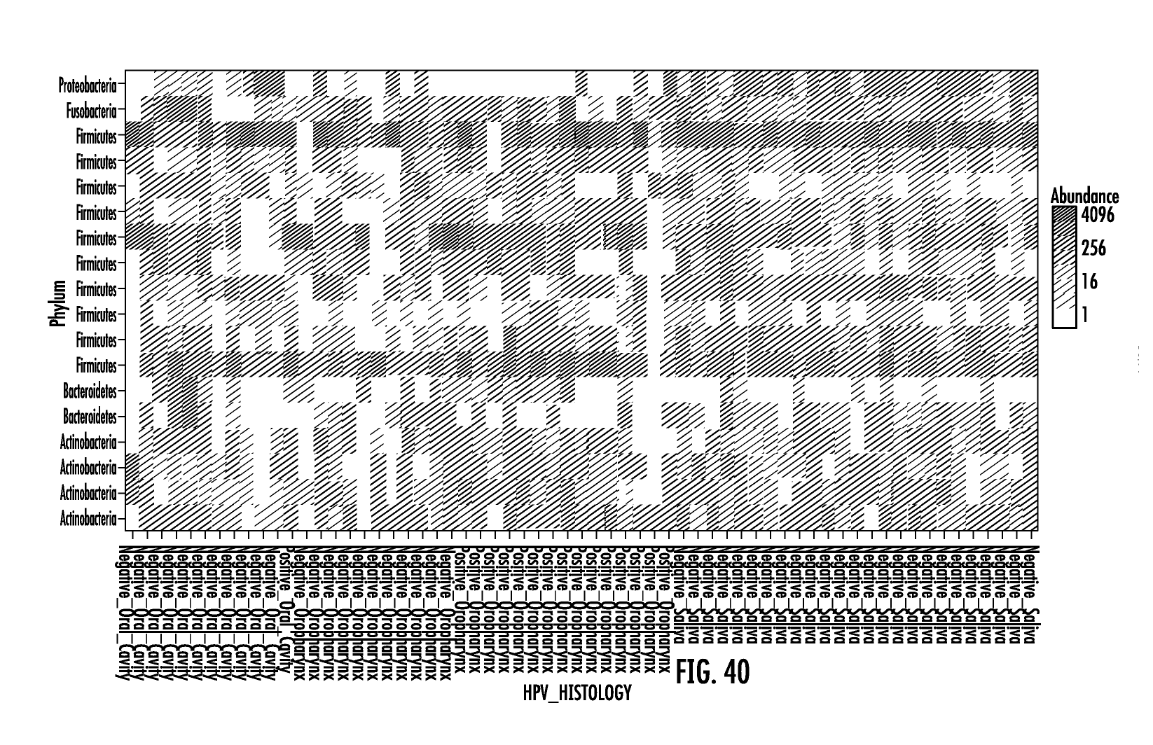

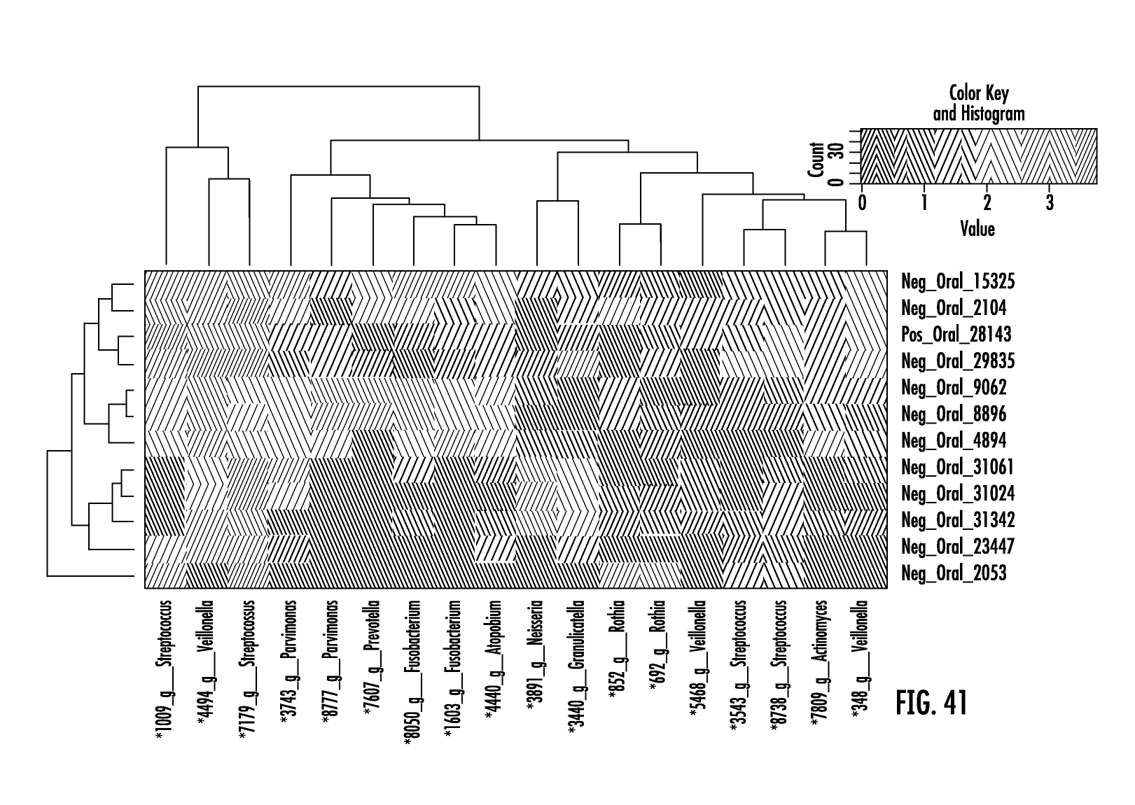

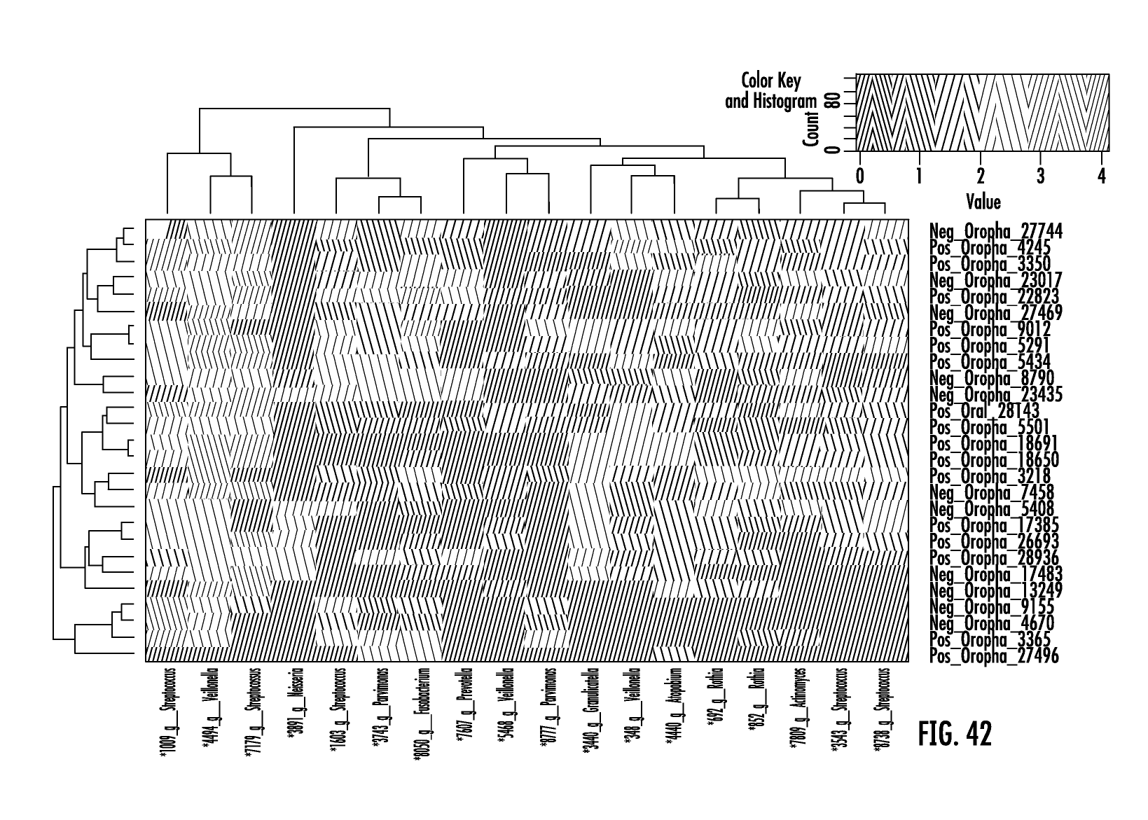

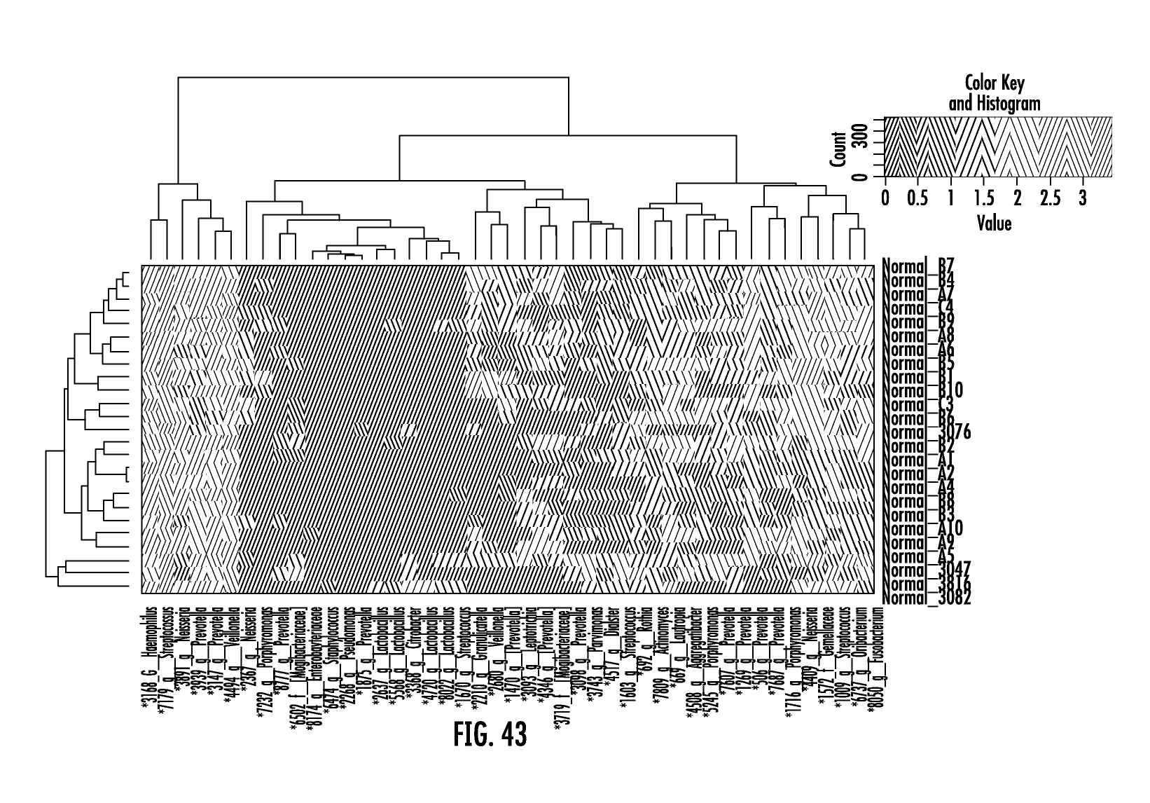

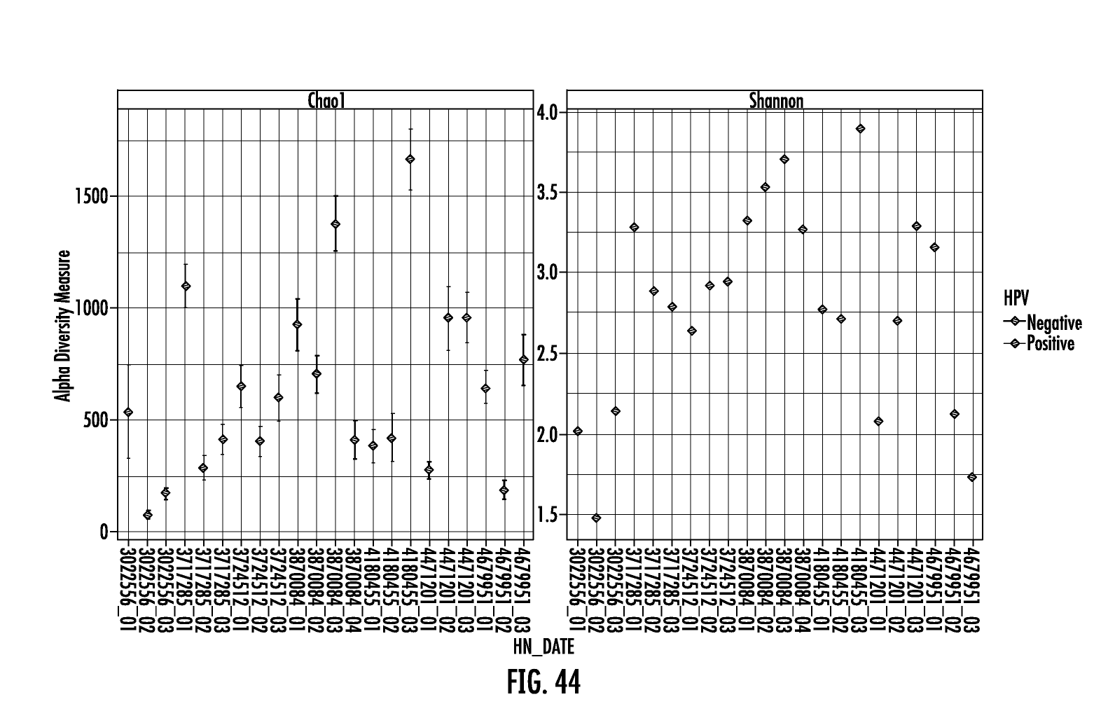

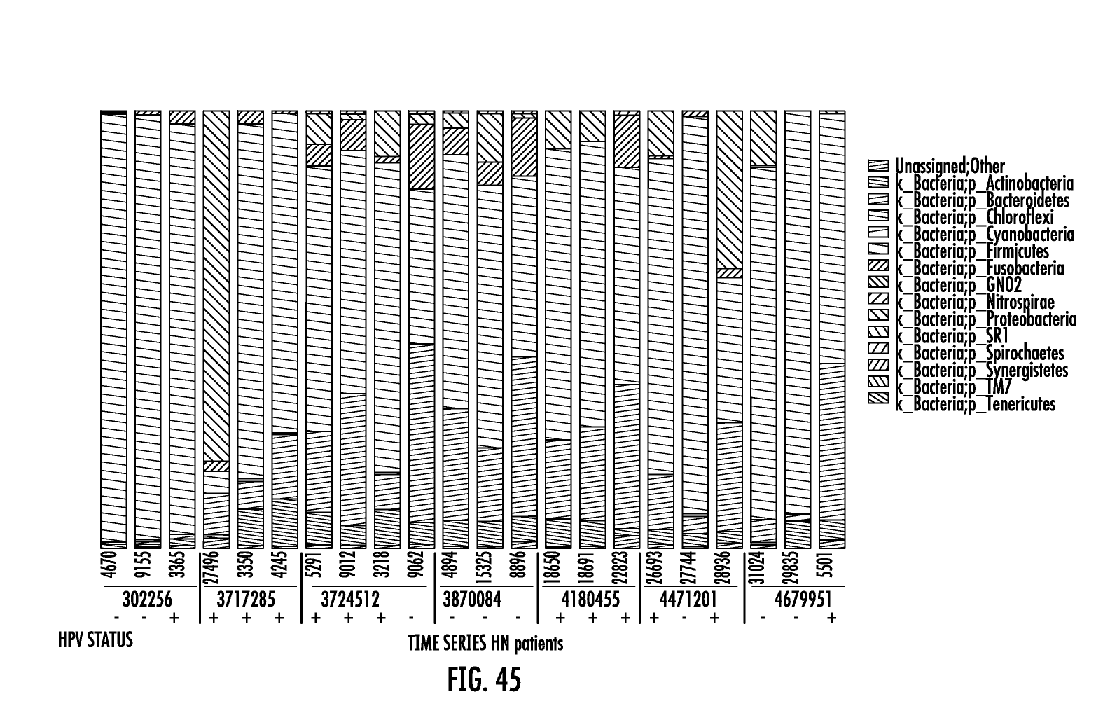

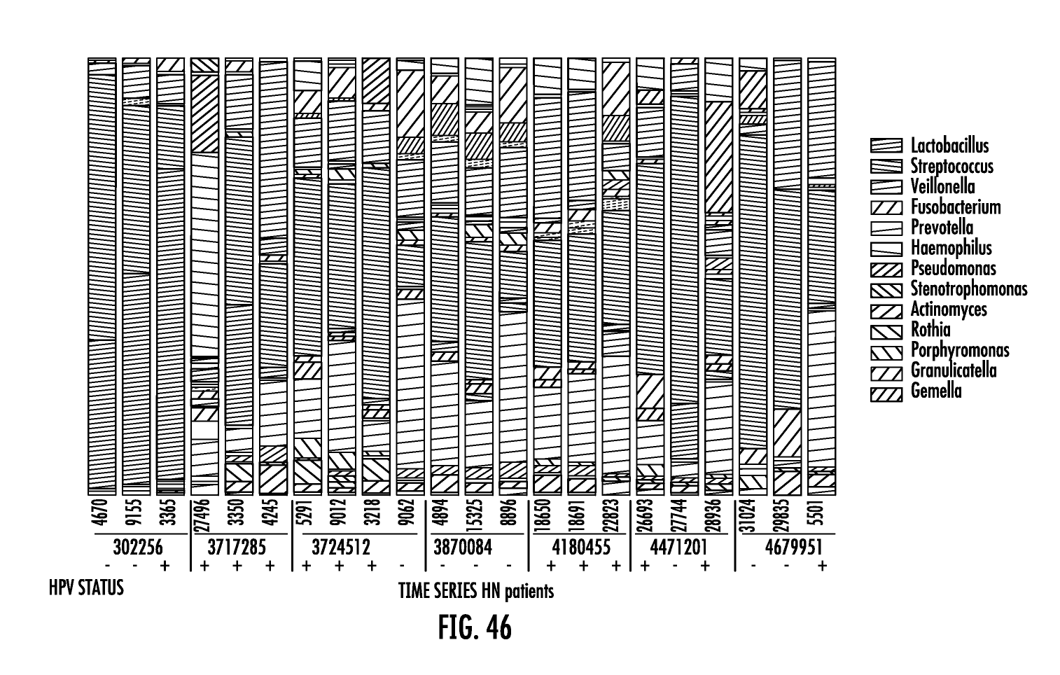

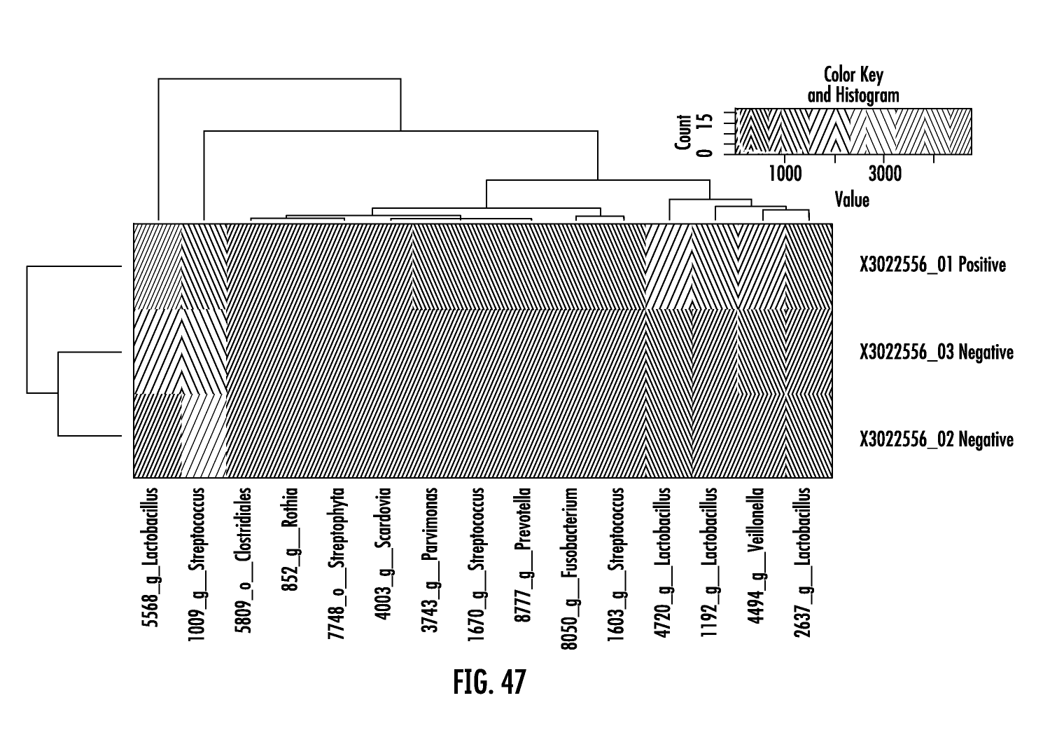

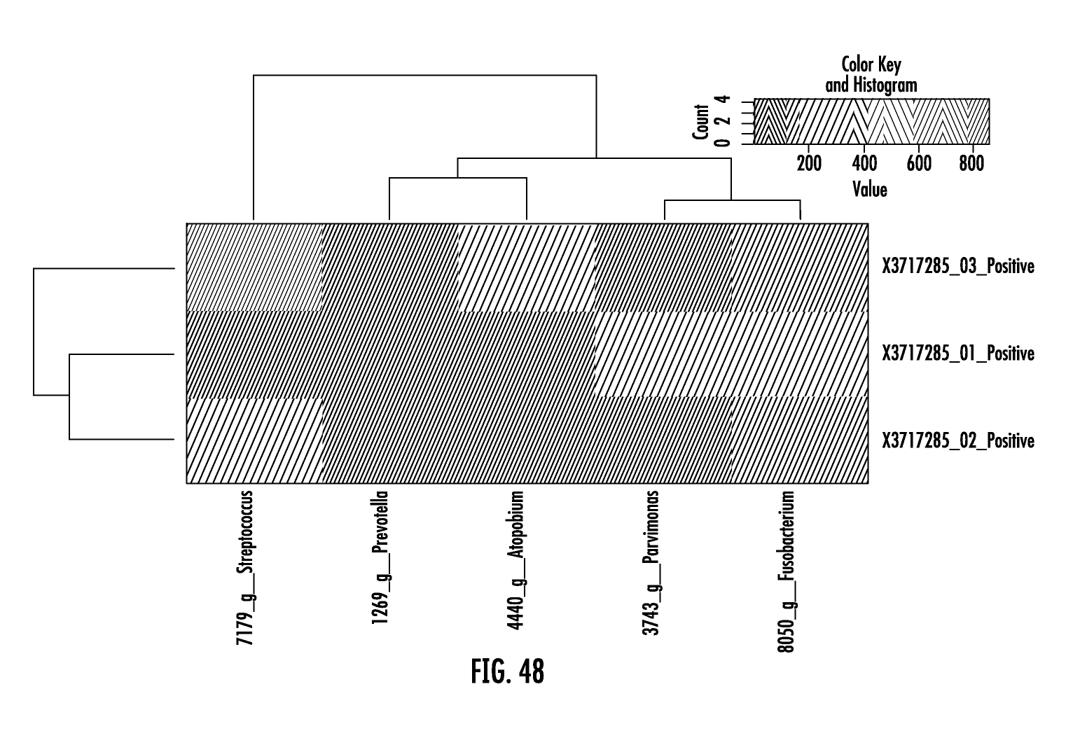

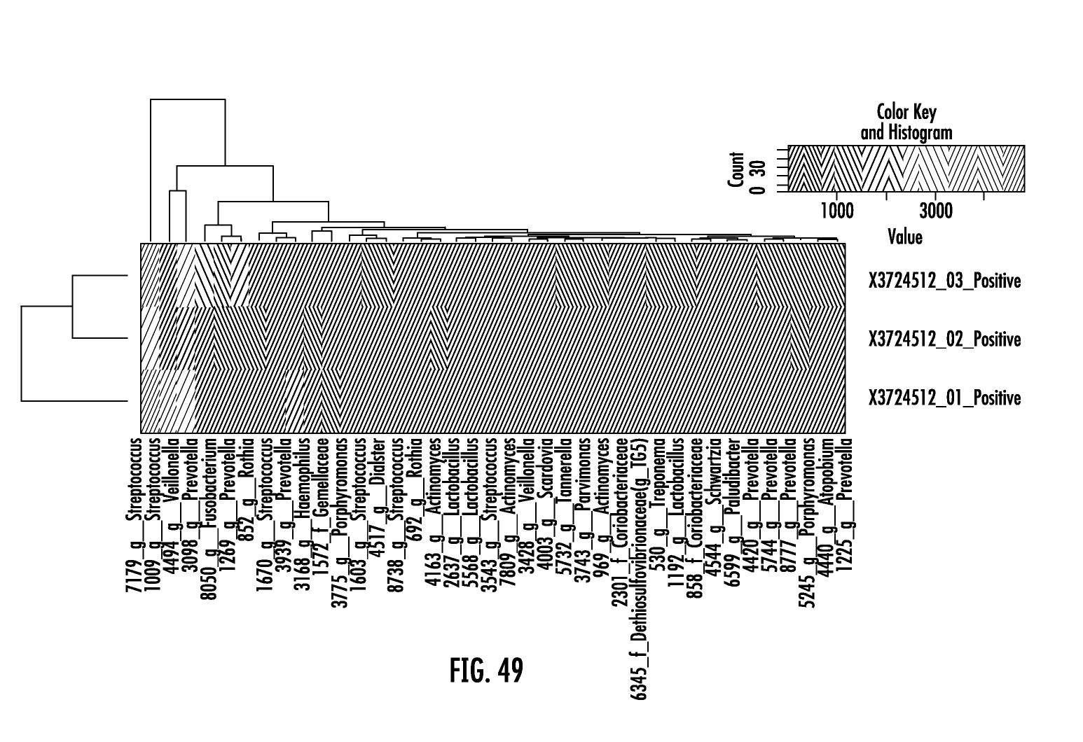

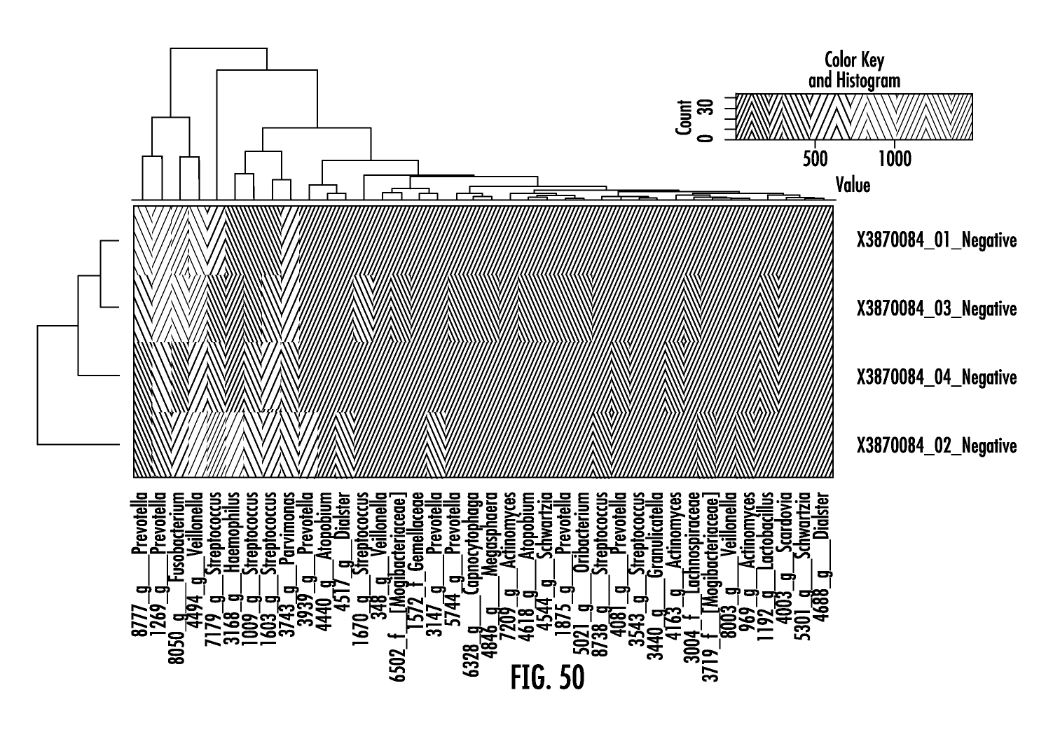

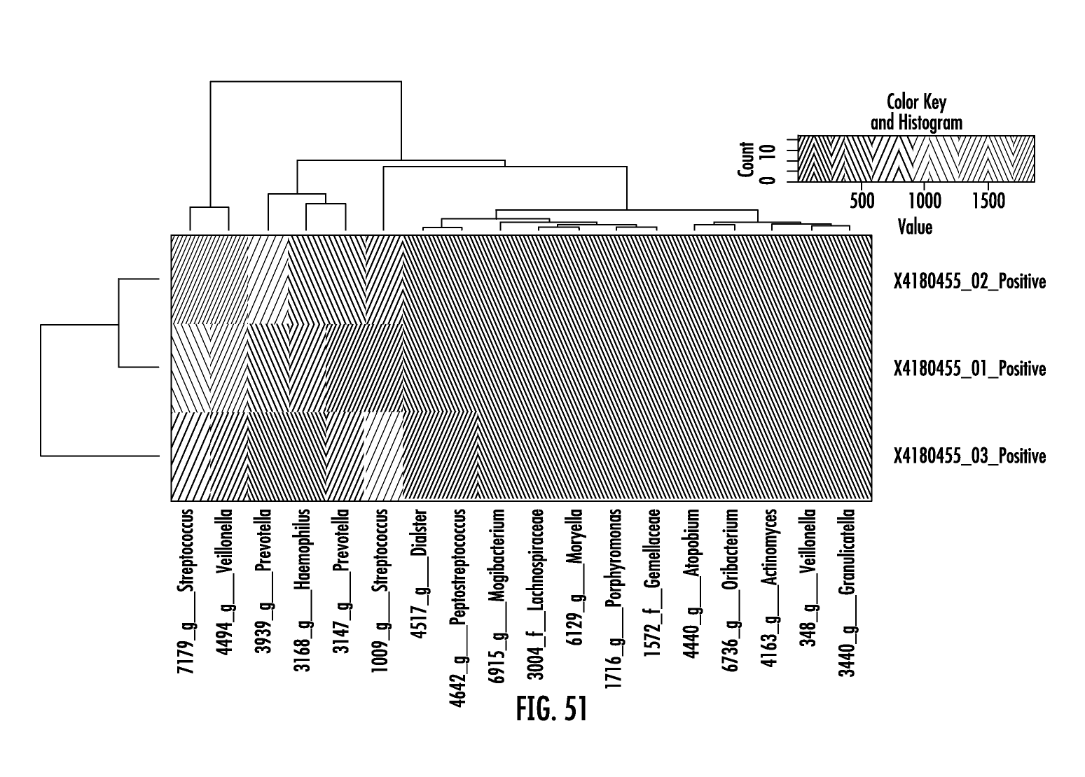

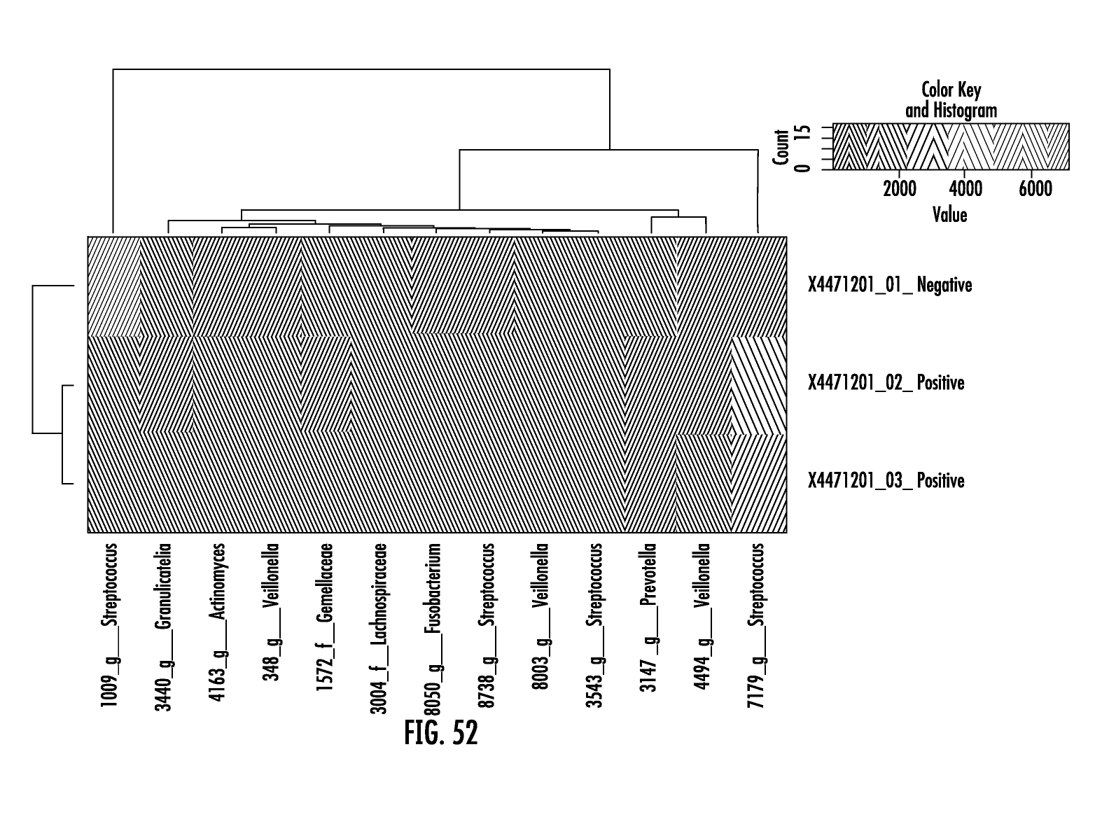

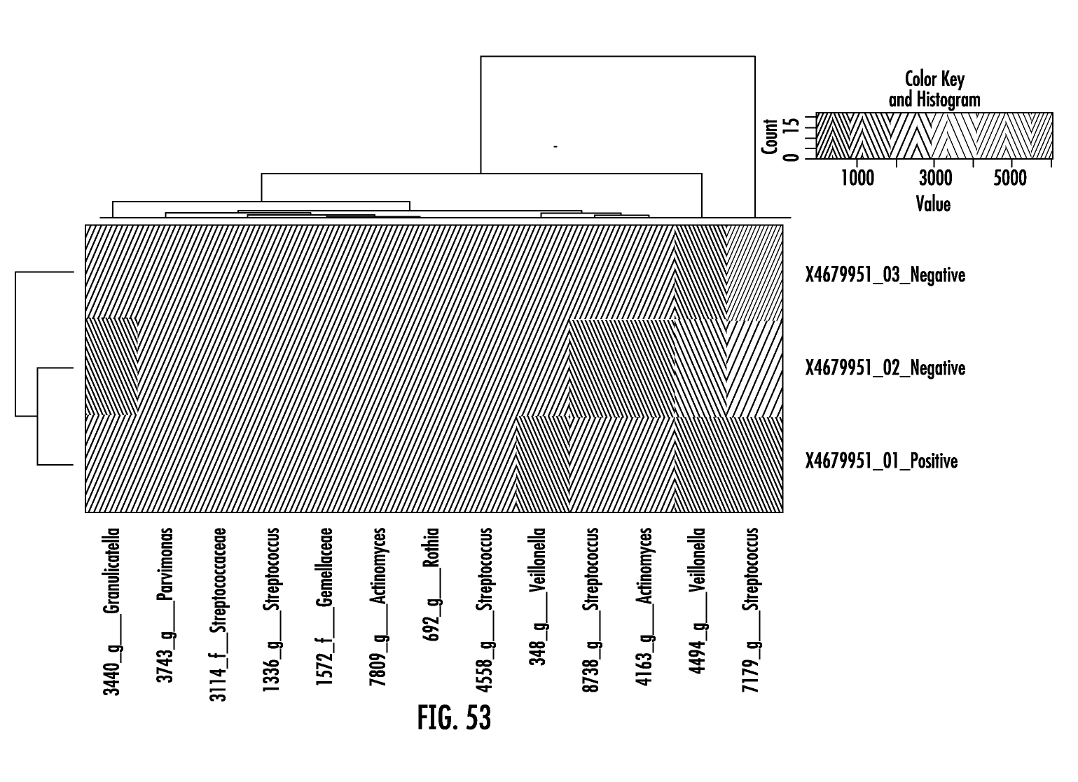

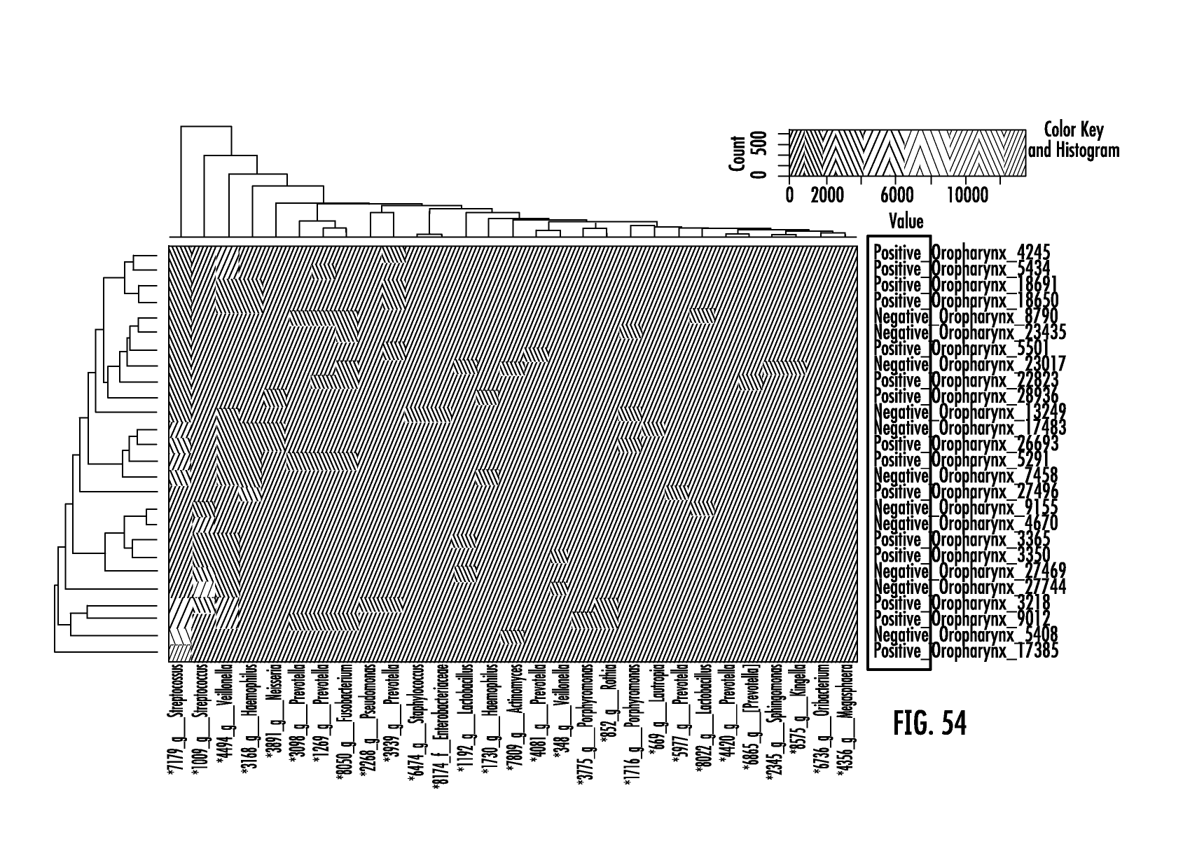

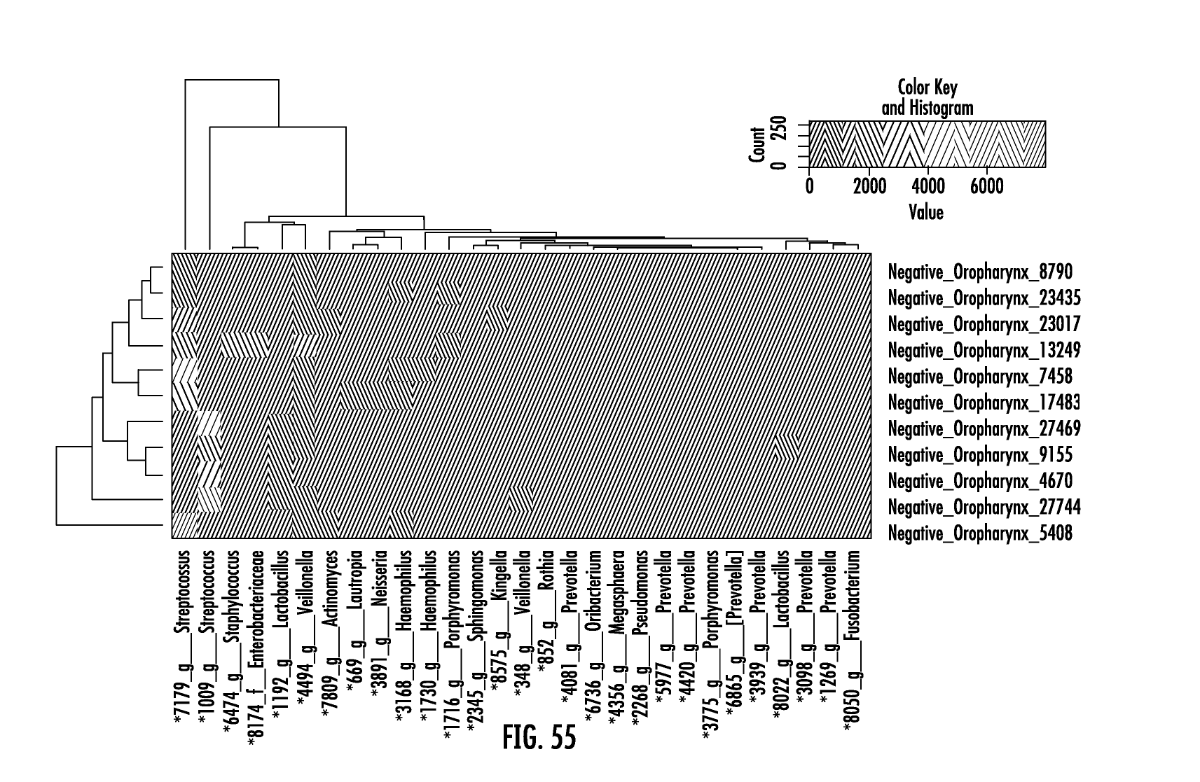

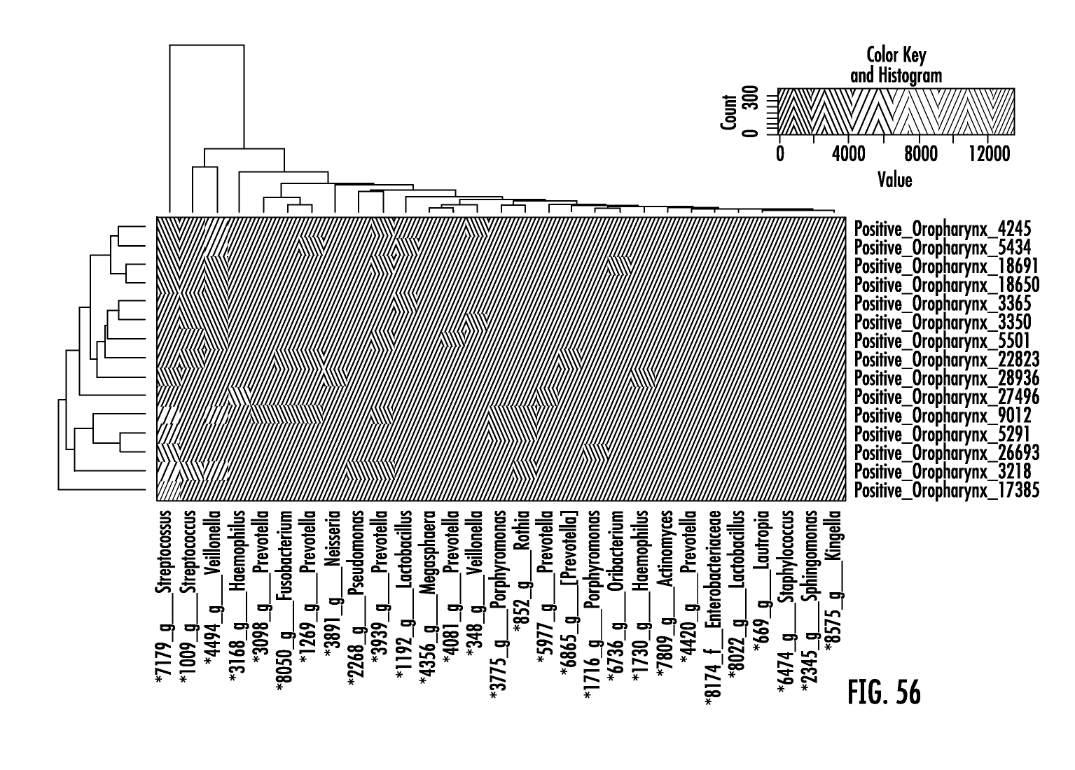

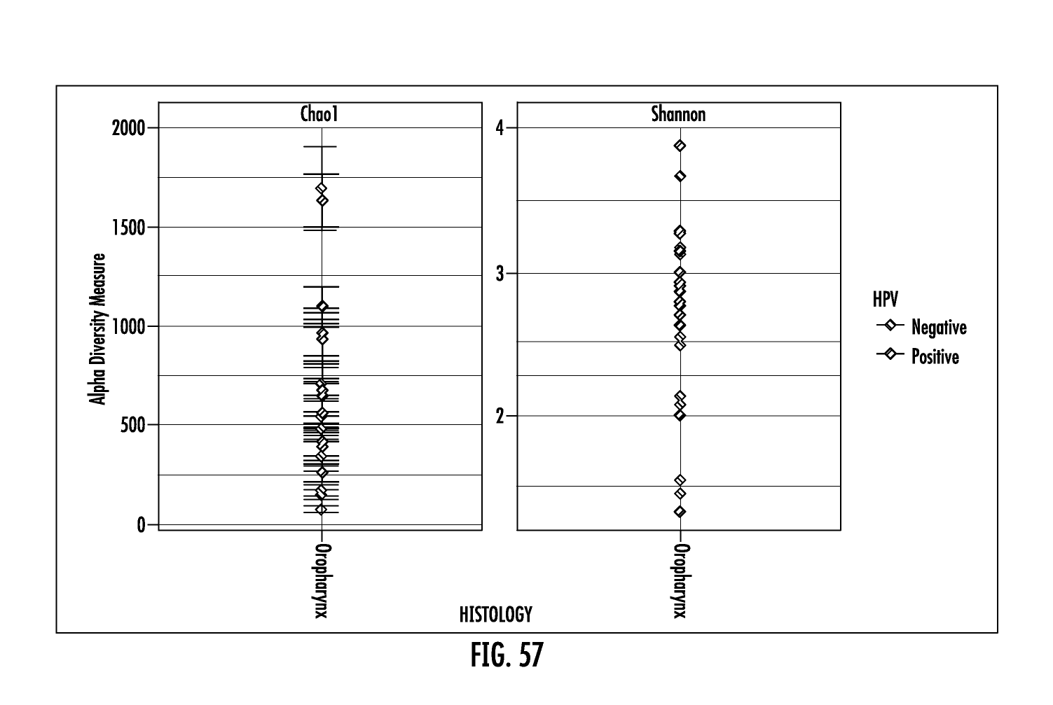

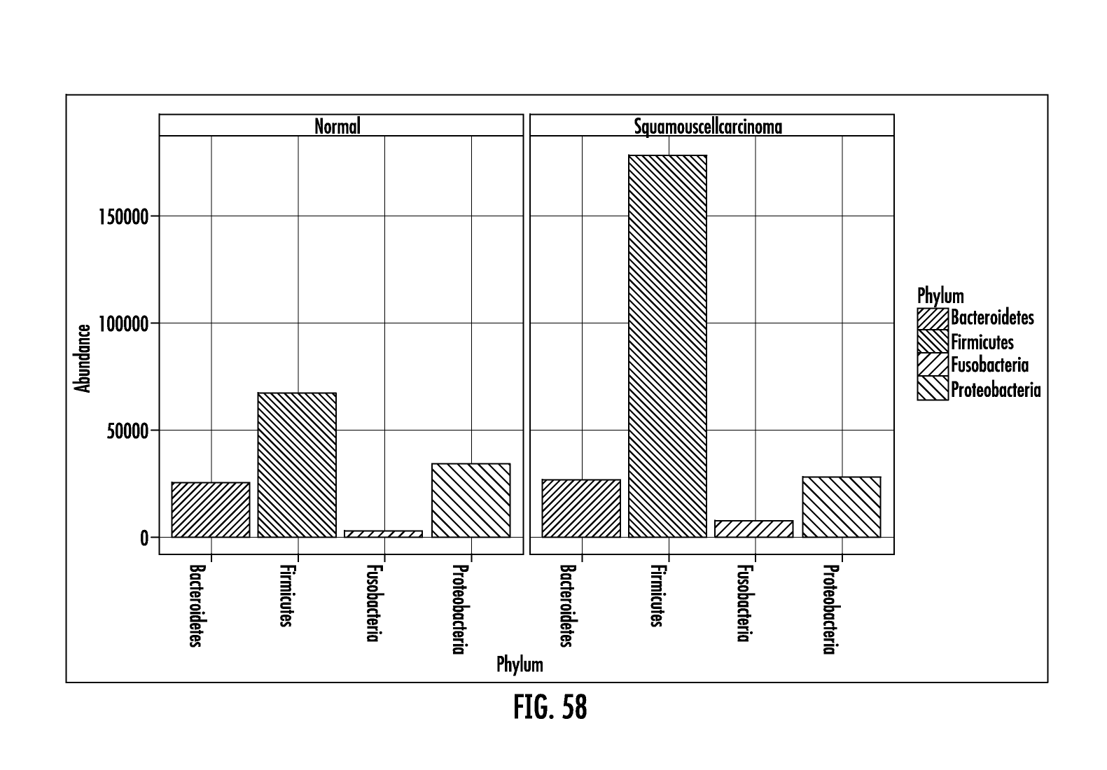

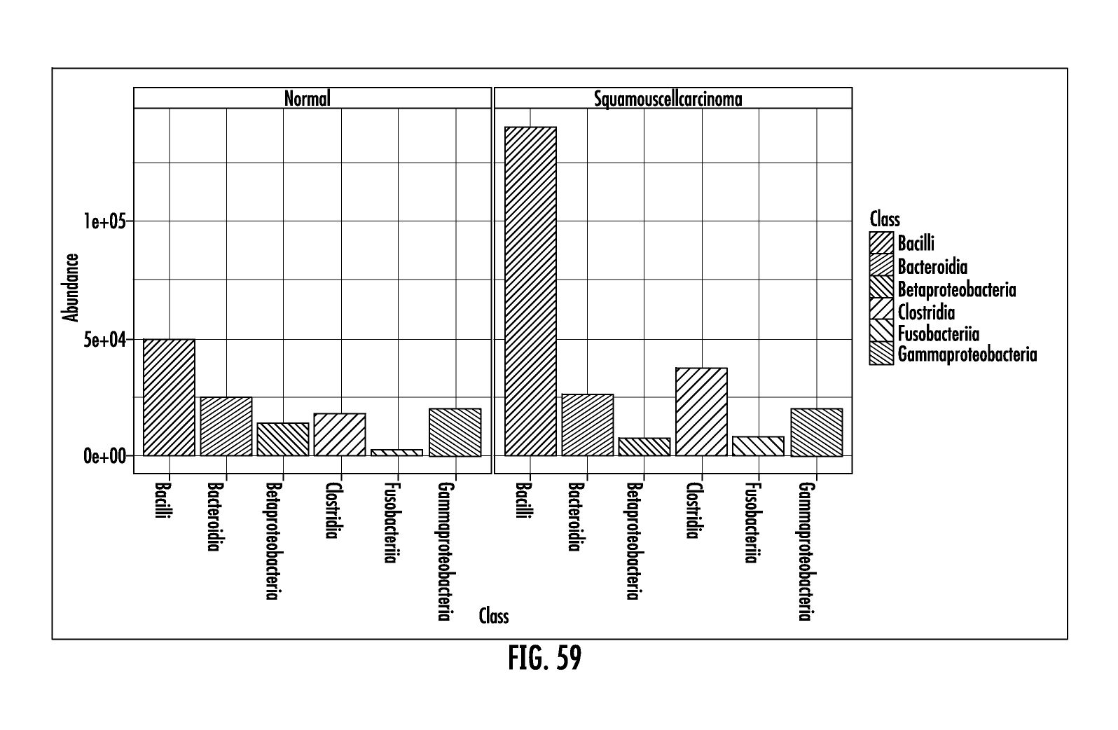

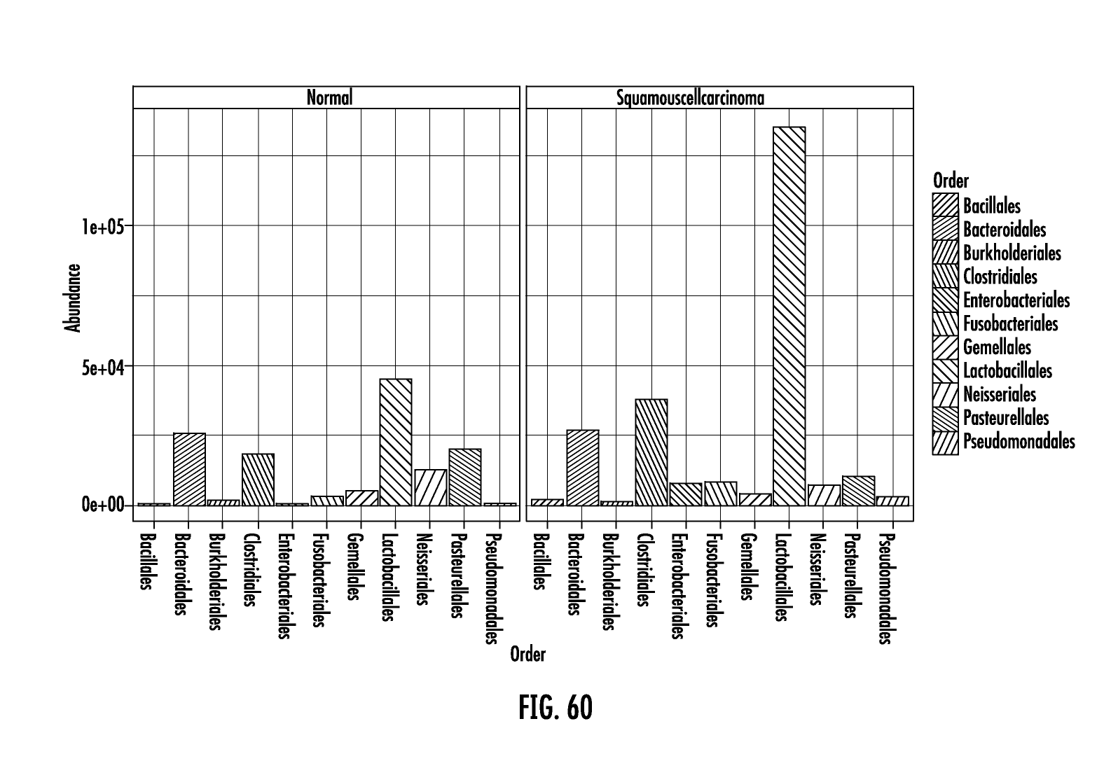

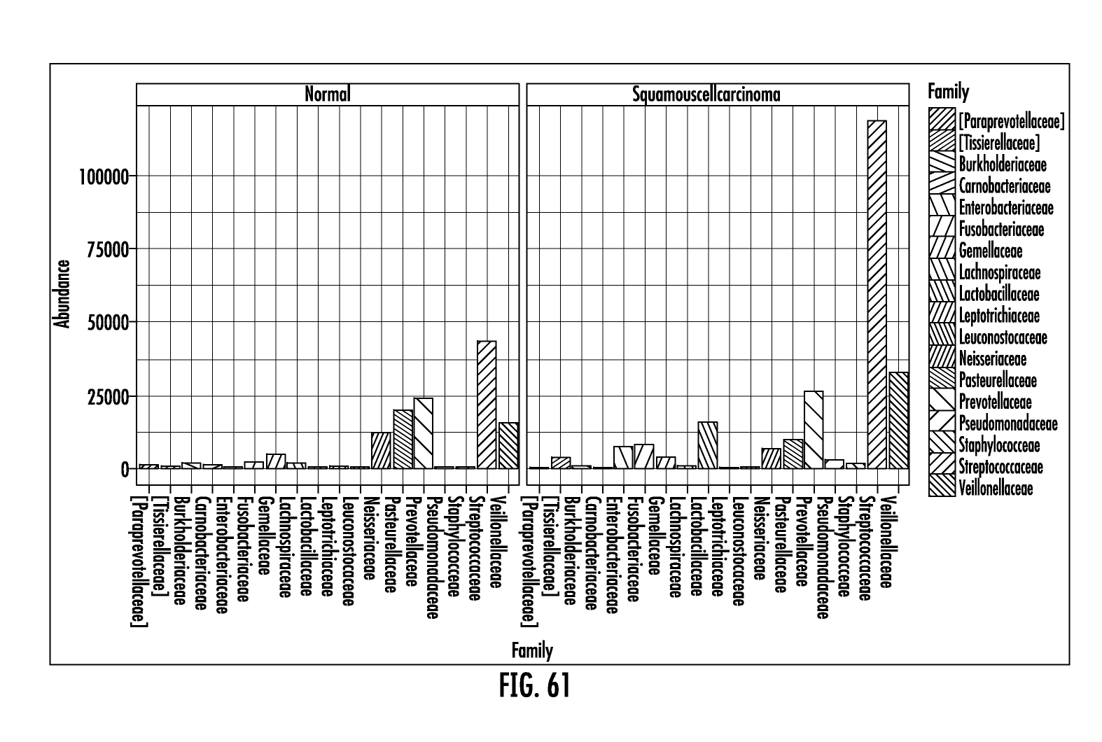

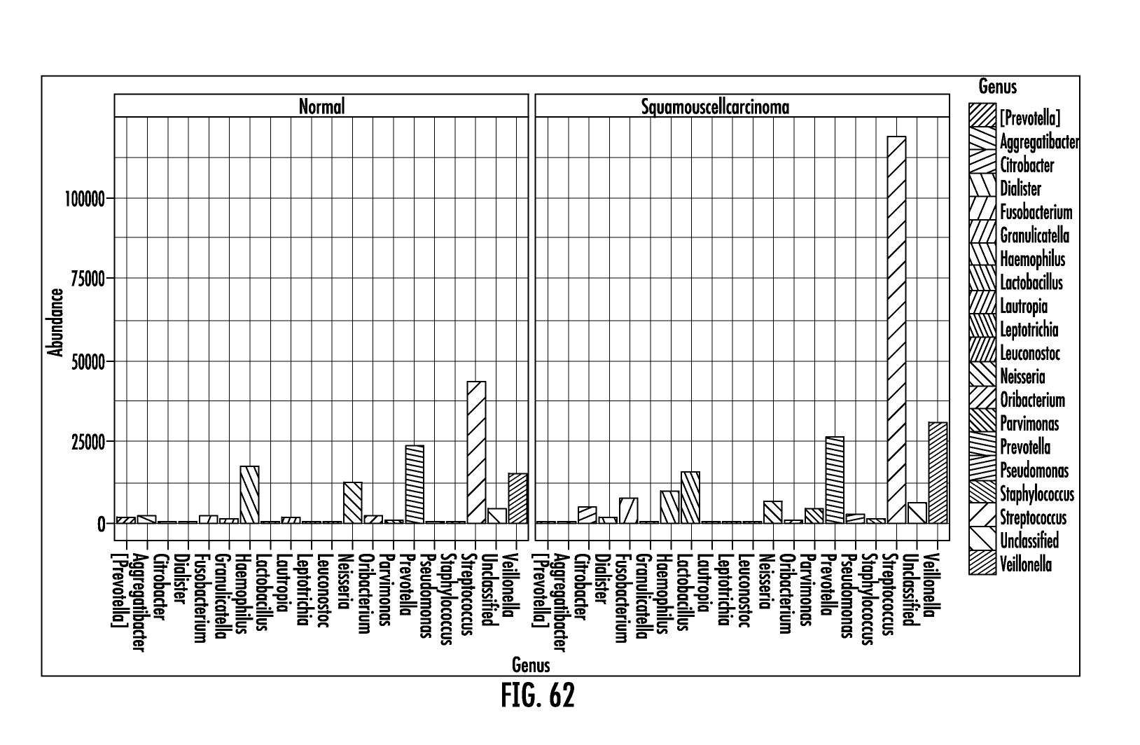

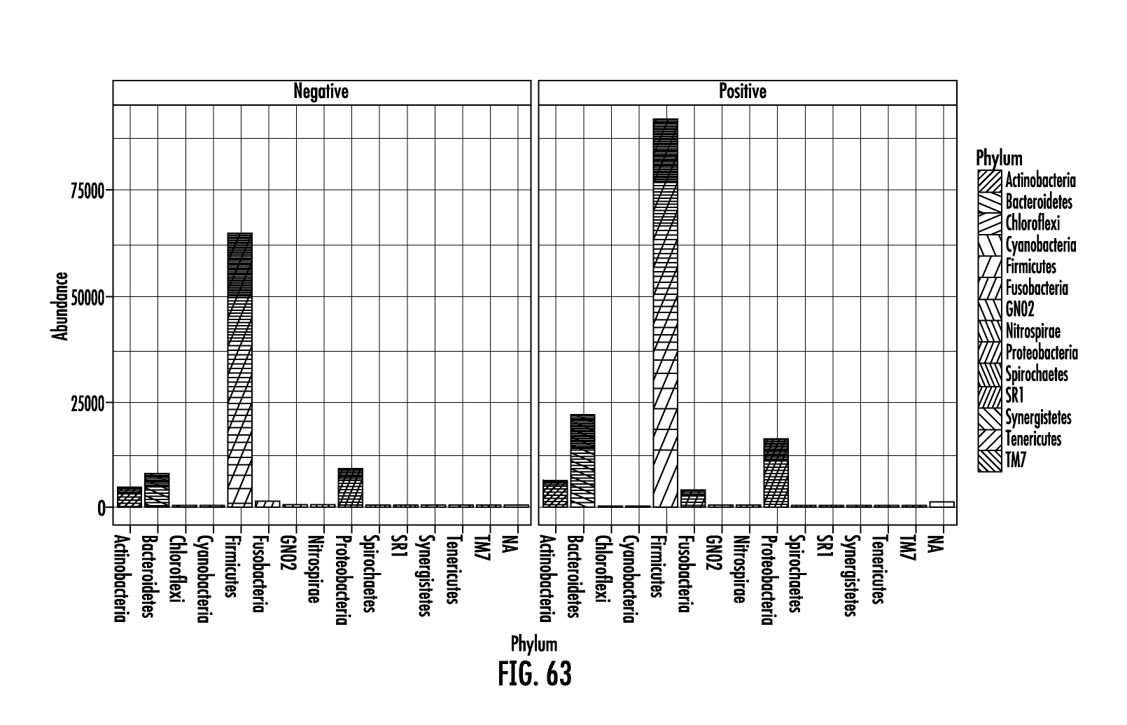

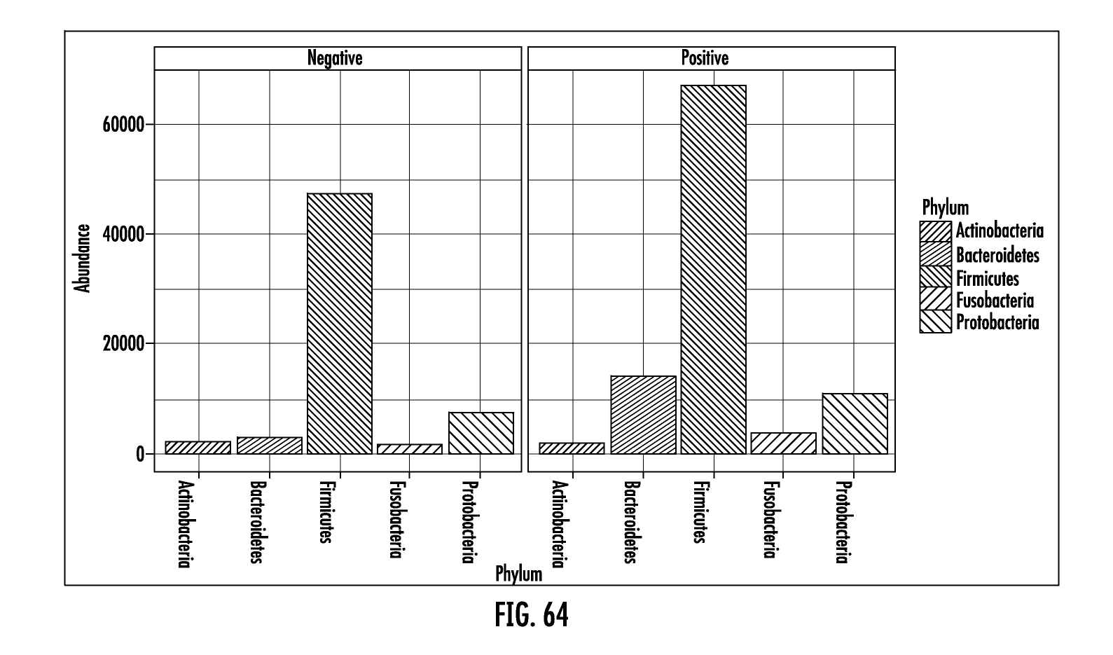

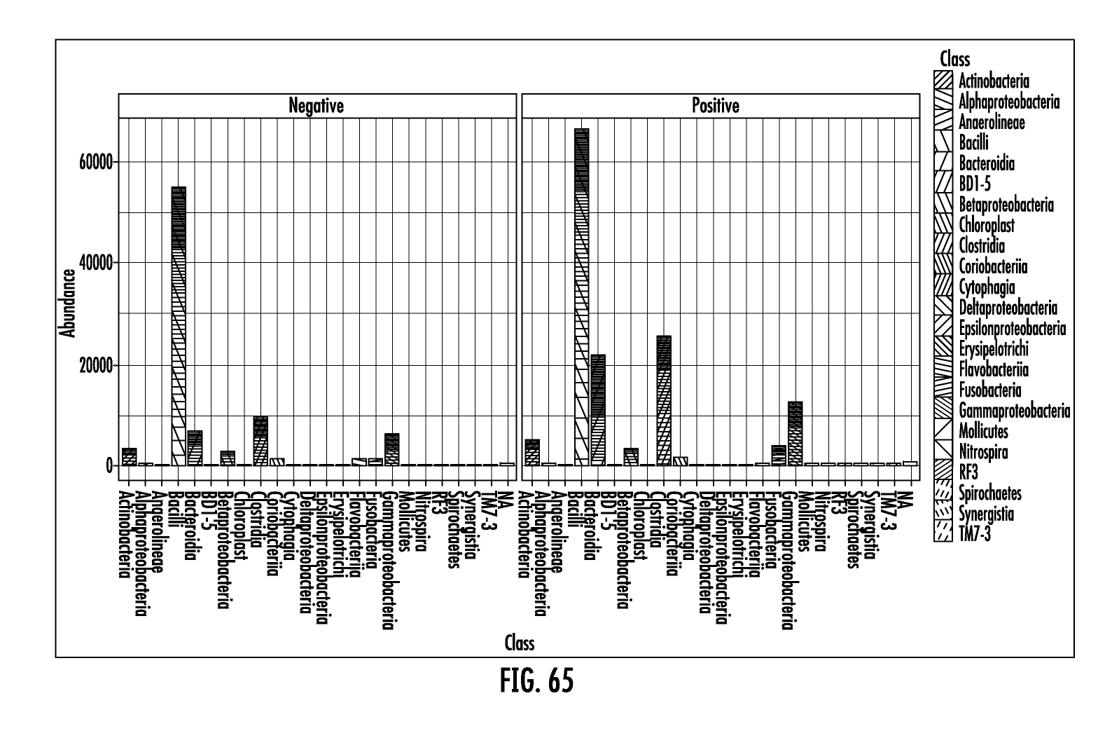

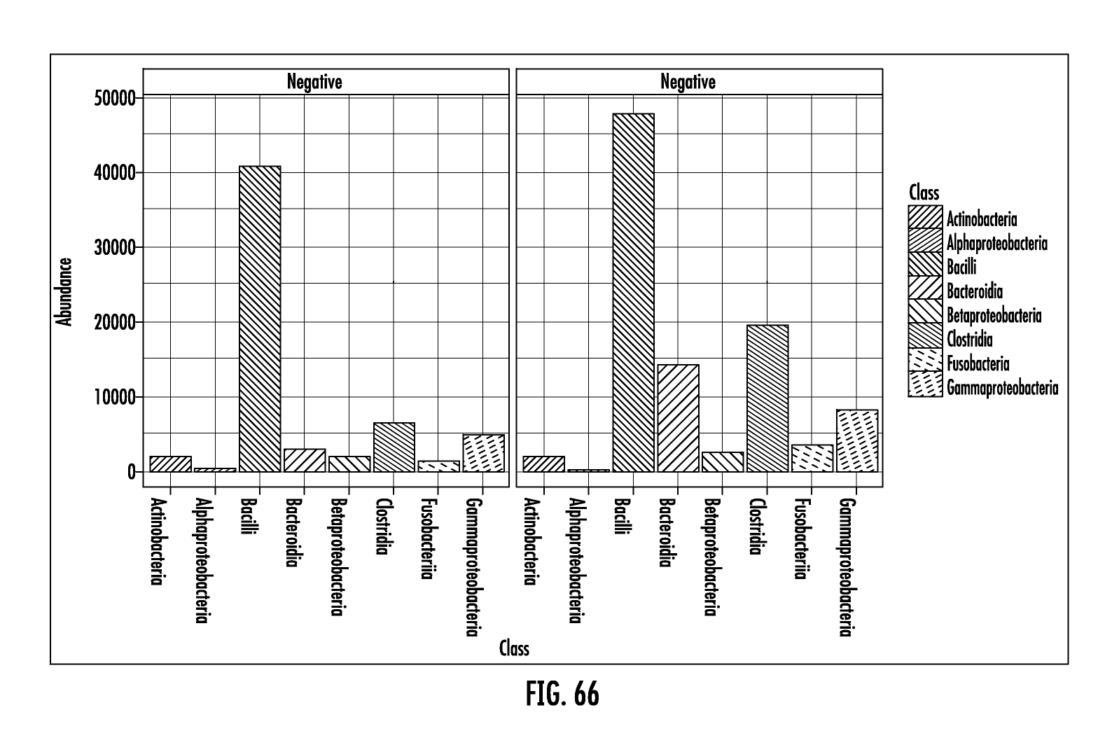

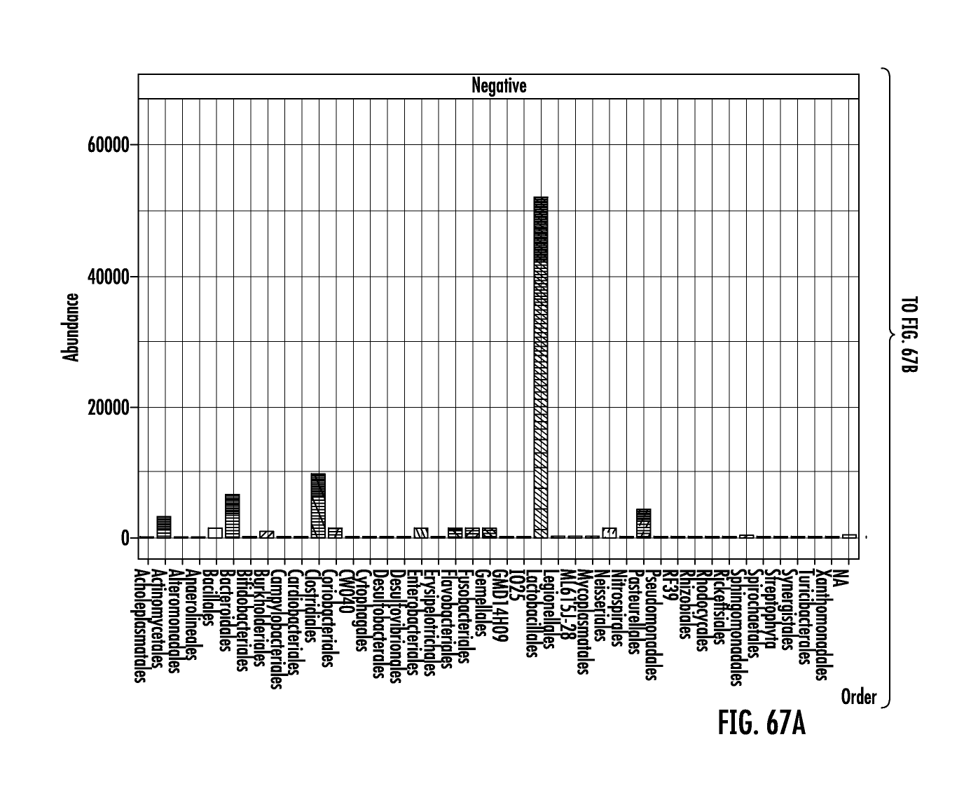

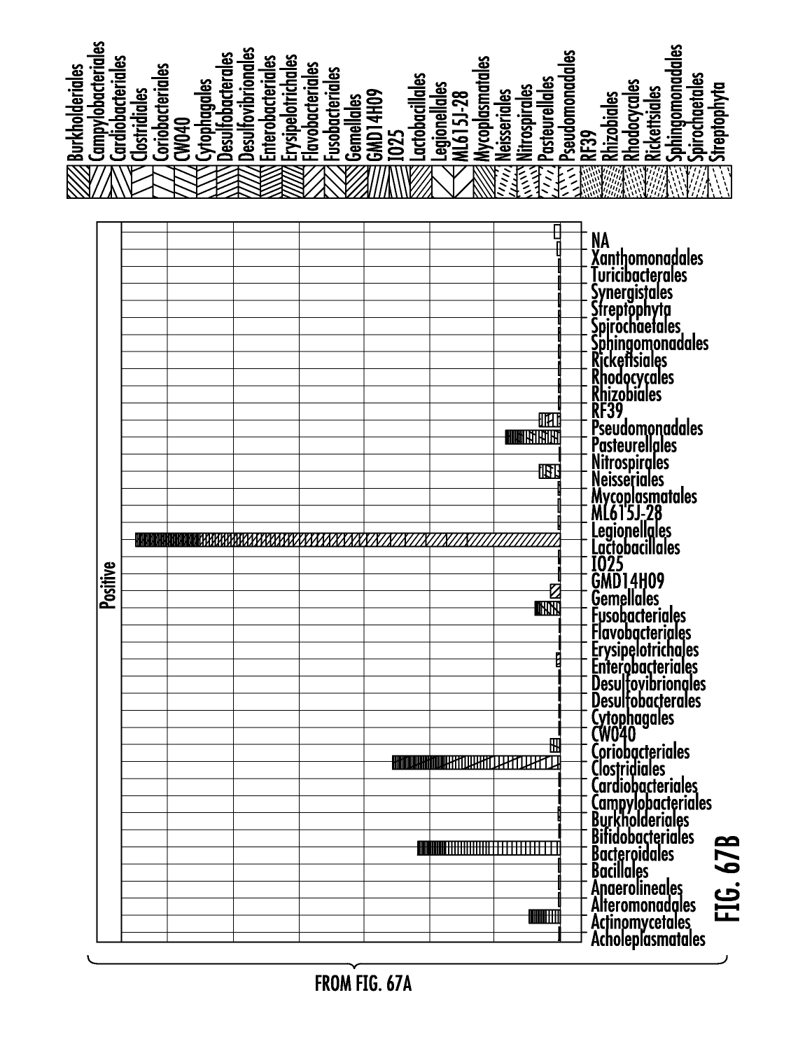

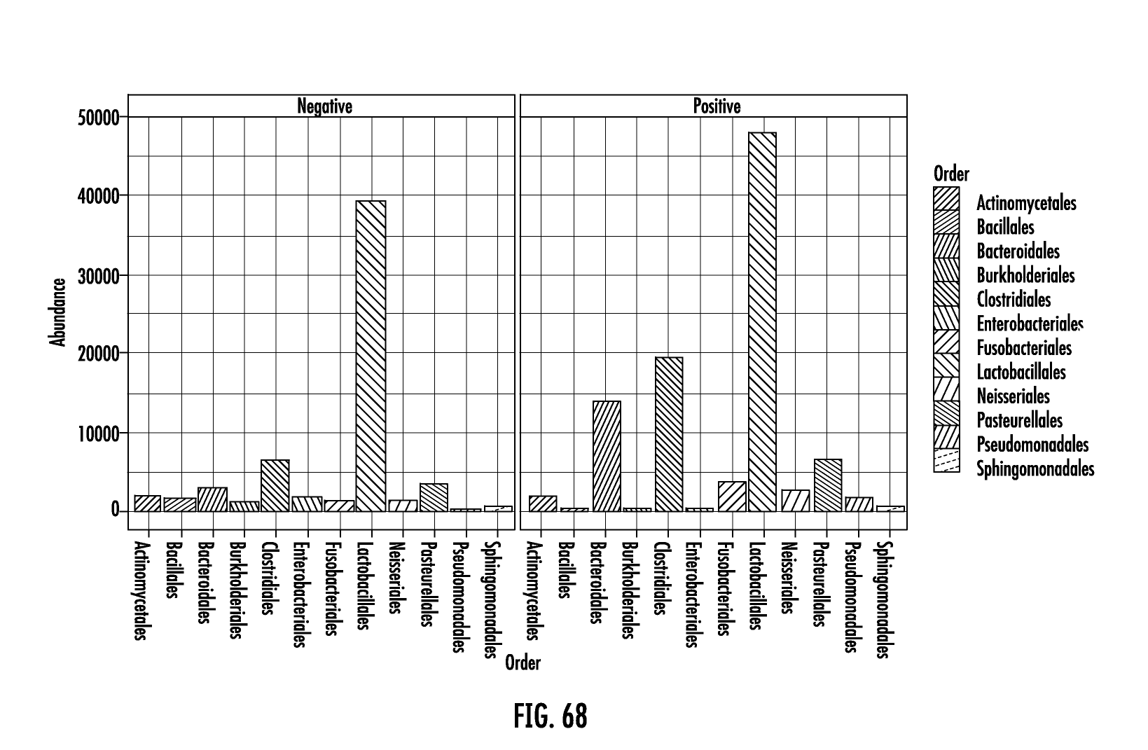

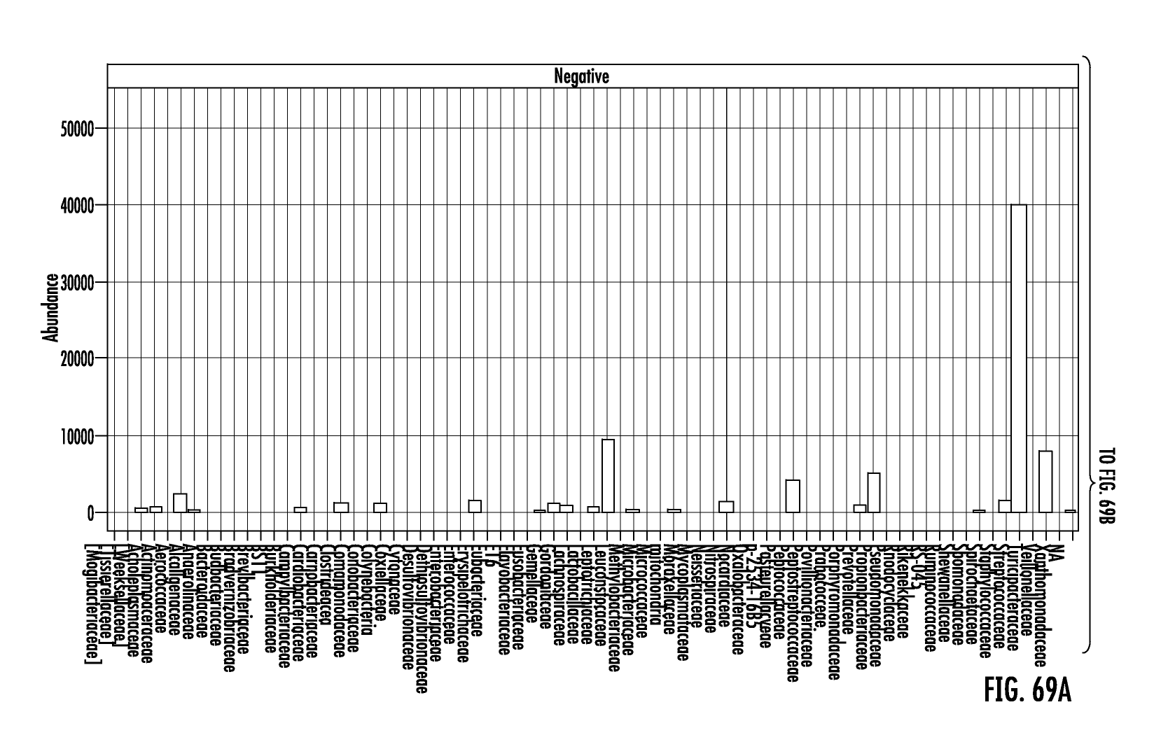

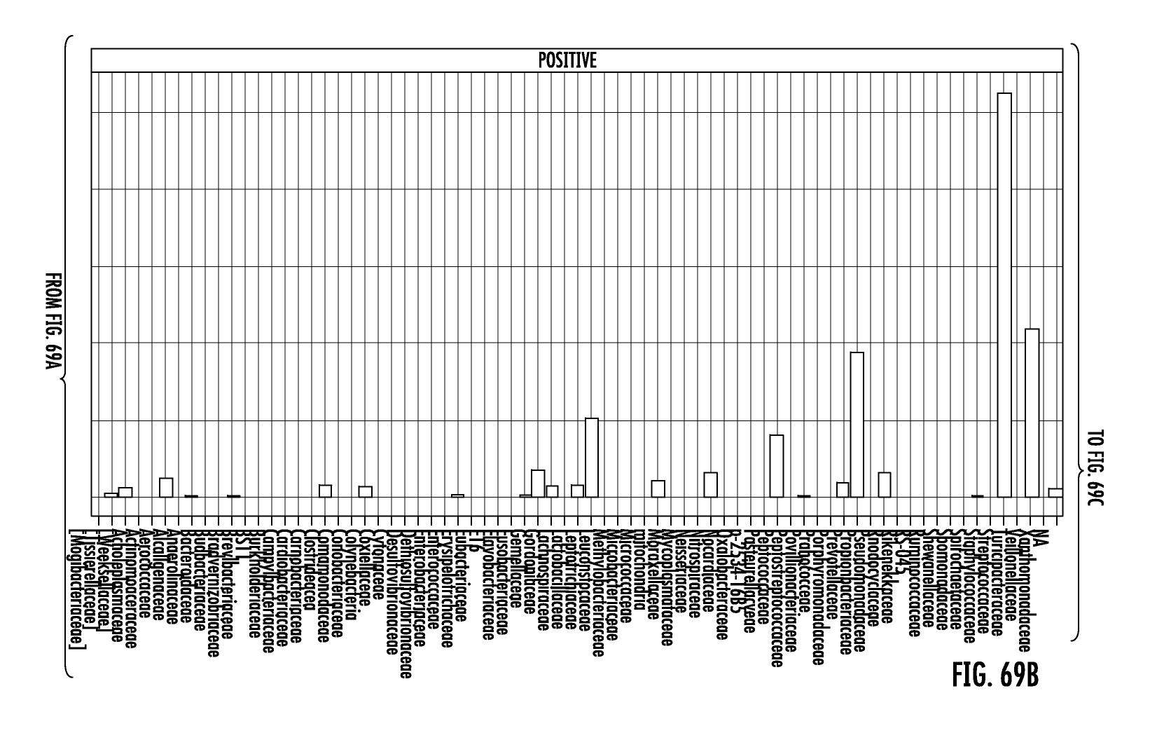

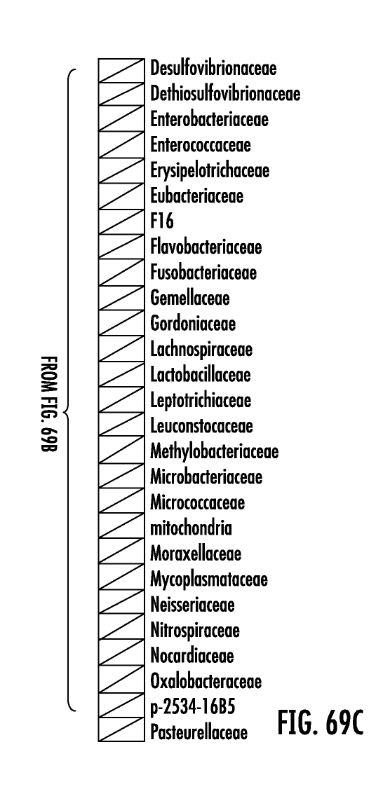

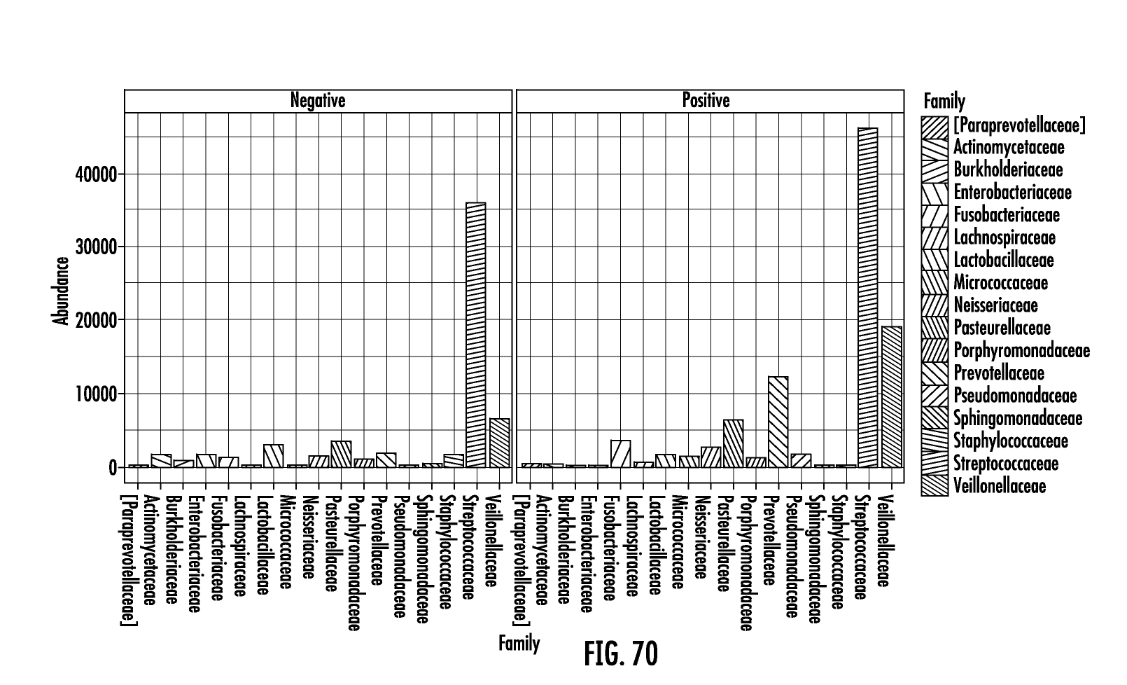

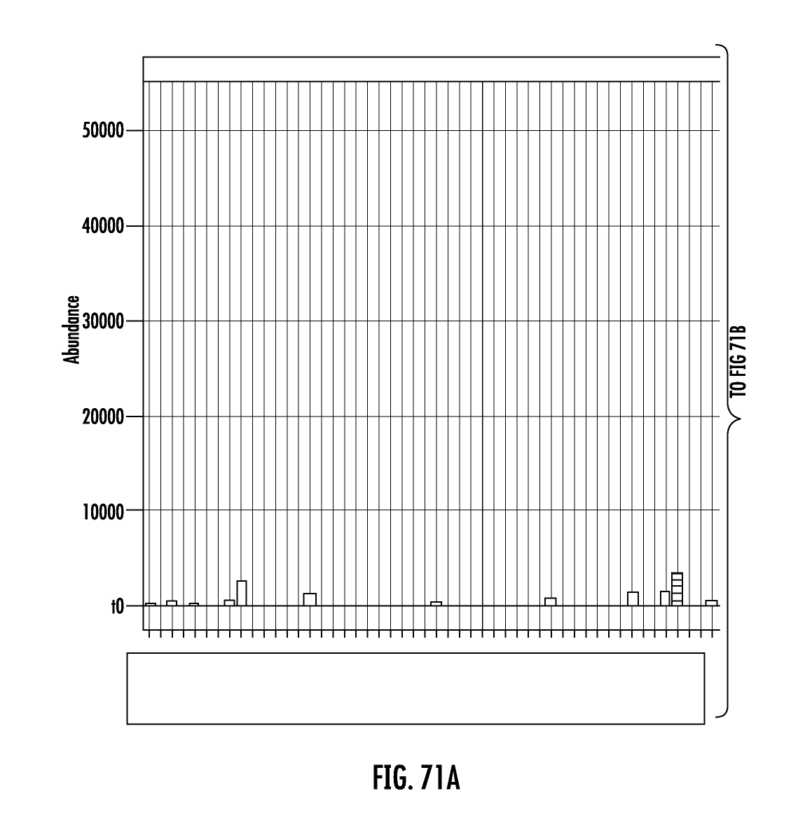

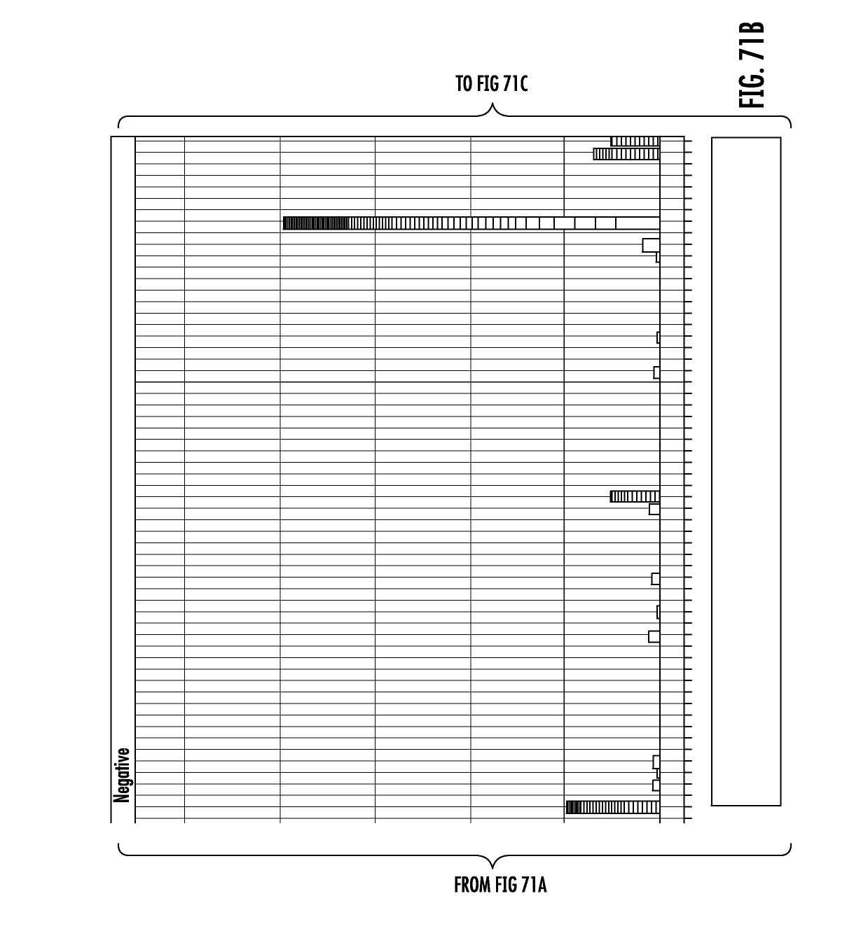

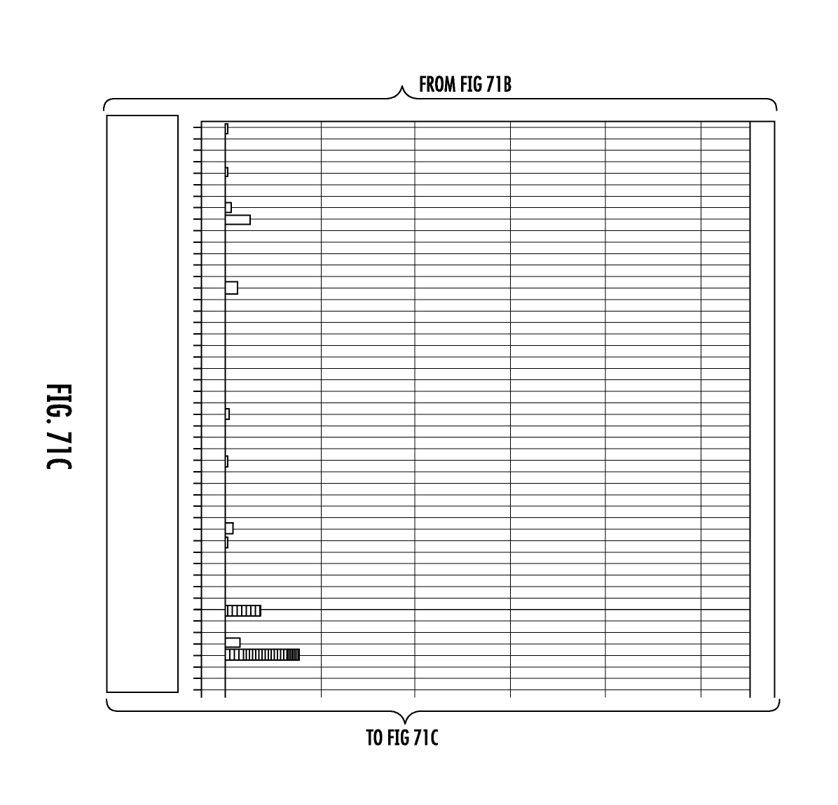

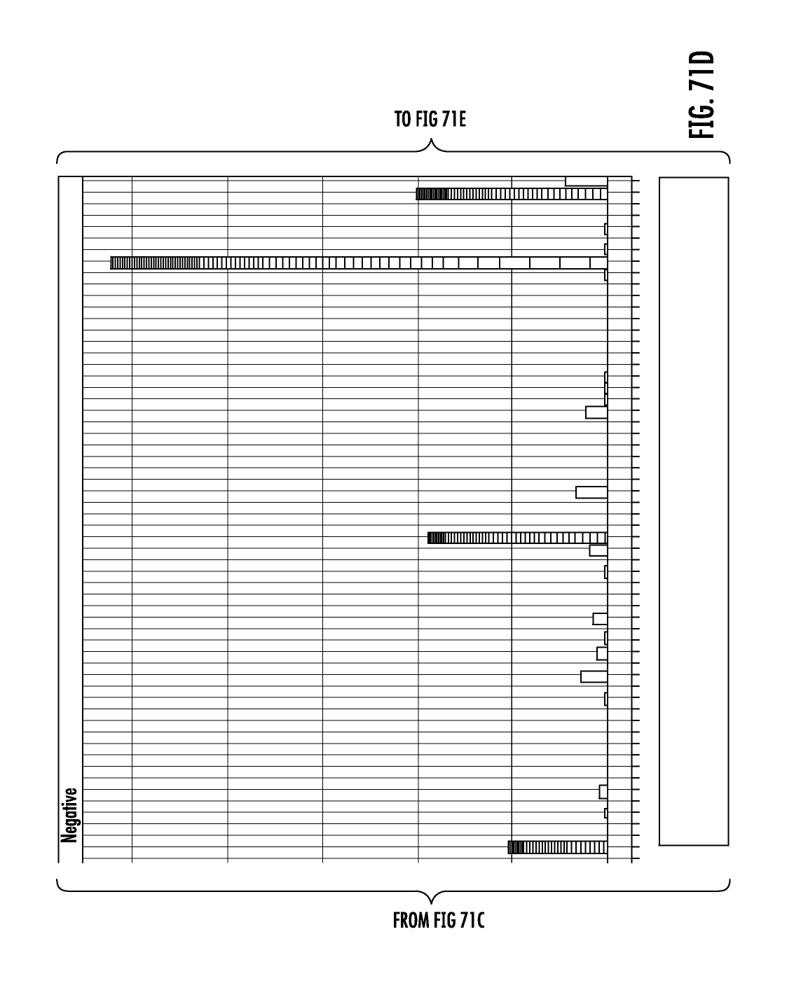

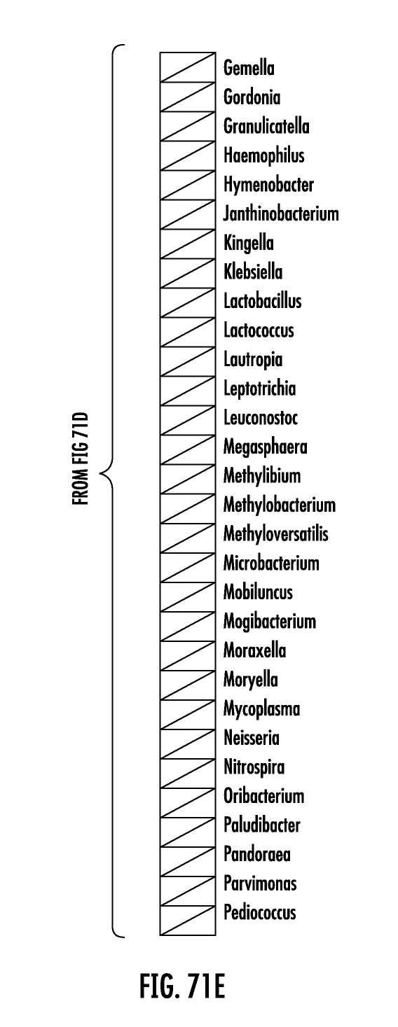

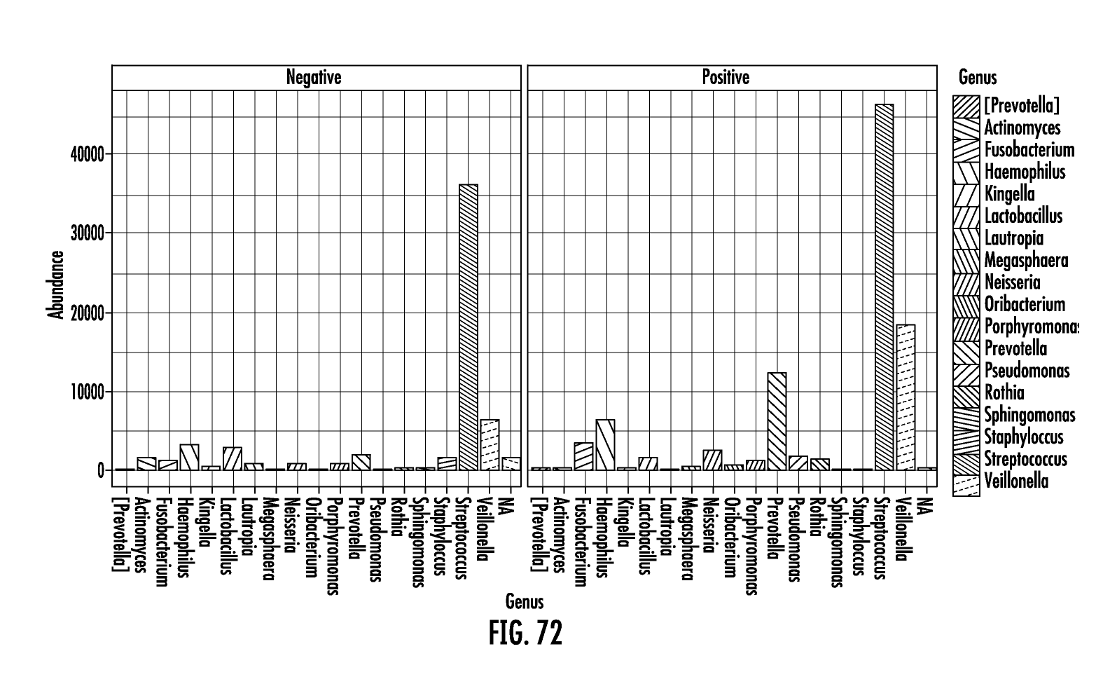

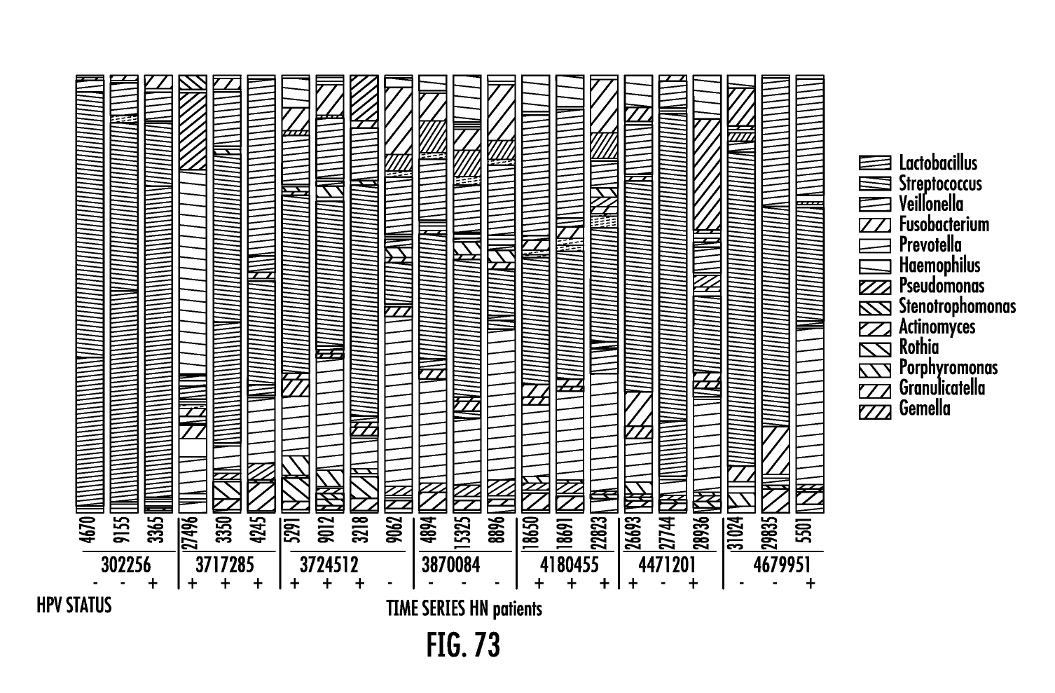

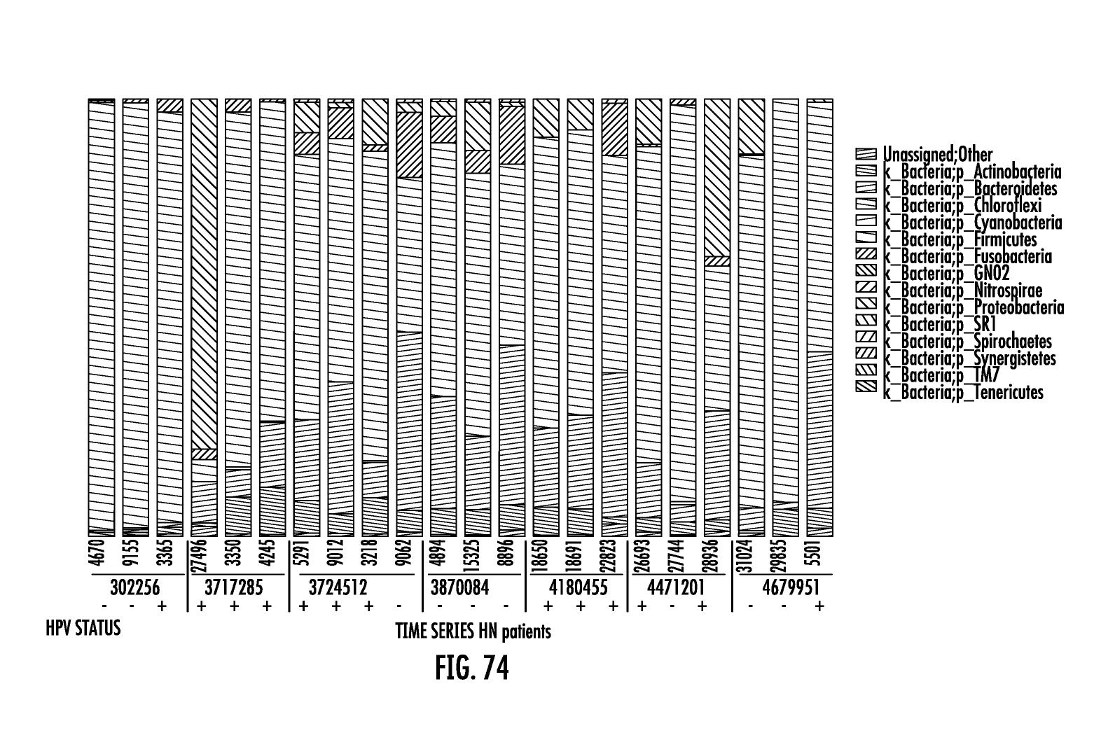

skiff.zip: This additional directory provides hierarchical clusterings of samples at different taxonomic levels with a heatmap overlay. Values that are analyzed by my program (called Skiff) are either proportions or log-normalized proportions—you can tell which from the filenames. As an example, the file: otu_table_even3594 phylum.lognormalized.pdf is an analysis at the phylum level of the samples, using log-transformed proportions as the values. FIG. 1 is an example at the order level (otu_table_even3594_order.lognormalized.pdf):

Unweighted.unifrac.pcoa.pdf: Principal coordinate analysis (beta-diversity) is also of interest in the 16S analysis community. A PCoA plot that uses the unweighted Unifrac distance metric is included, and displays clustering of samples colored by cancer and HPV status (OC HPV+ in blue, OC HPV− in green, Normal in Red). See FIG. 2.

### Example 2: Analysis of 16S rRNA Amplicon Sequences Associated with Head and Neck Squamous Cell Carcinoma (HNSCC) and Human Papilloma Virus (HPV) Infections

This example is based on a concatenation of 6 batches that contained 63 multiplexed samples.

TABLE 1

| HPV Tumor Site | Number Of Samples |
|---|---|
| Negative_Normal | 25 |
| Negative_Oral_Cavity | 11 |
| Positive_Oral_Cavity | 1 |
| Negative_Oropharynx | 11 |
| Positive_Oropharynx | 15 |
| Total | 63 |

### See FIG. 40.

TABLE 2
Number of Samples per OSCC (+/−) Status

| Row Labels | Number of Samples |
|---|---|
| Normal epithelium | 25 |
| (HNSCC+) | 38 |
| Squamous cell | |
| carcinoma | |
| Sample Total | 63 |

TABLE 3
Number of Samples per Age Range Categories

| Age Range | Number of Samples |
|---|---|
| OVER70 | 5 |
| RANGE30-50 | 11 |
| RANGE50-70 | 29 |
| UNDER30 | 18 |
| Grand Total | 63 |

TABLE 4
Number of Samples per Gender

| Gender | Number of Samples |
|---|---|
| F | 21 |
| M | 42 |
| Grand Total | 63 |

TABLE 5
Number of Samples per HPV Status

| HPV Status | Number of Samples |
|---|---|
| Negative | 47 |
| Positive | 16 |
| Grand | 63 |
| Total | |

TABLE 6
Number of Samples per Sample Histology

| Histology | Number of Samples |
|---|---|
| Oral Cavity | 12 |
| SCC | |
| Oropharynx | 26 |
| SCC | |
| Normal | 25 |
| Grand Total | 63 |

TABLE 7
Number of Samples per HPV-OC Status

| HPV-OC Status | Number of Samples |
|---|---|
| Negative Normal | 25 |
| Negative Squamous cell carcinoma | 22 |
| Positive Squamous cell carcinoma | 16 |
| Grand Total | 63 |

Preprocessing Stats:

TABLE 8
Number of Sequences per Batch

| Data | Batch #1 | Batch #2 | Batch #3 | Batch #4 | Batch #5 | Batch #6 | Combined |
|---|---|---|---|---|---|---|---|
| Number of Samples | 8 | 4 | 14 | 13 | 12 | 12 | 63 |
| Number of Seqs Raw | 86900 | 73462 | 123527 | 123658 | 106062 | 94708 | 608317 |
| Ave Seqs Length | 519 | 522 | 522 | 524 | 523 | 521 | 522 |
| Ave Seqs Per Sample | 9102 | 17001 | 5697 | 8790 | 8209 | 7214 | 9335 |
| Number Seqs written | 72817 | 68003 | 79758 | 114269 | 98506 | 86570 | 519923 |

TABLE 9
Sequence Counts per Sample

| Sample ID | Sequence Count |
|---|---|
| 2053 | 7881 |
| 2104 | 8297 |
| 3047 | 19245 |
| 3076 | 5647 |
| 3082 | 8752 |
| 3218 | 17616 |
| 3350 | 3487 |
| 3365 | 10798 |
| 3816 | 8233 |
| 4245 | 7858 |
| 4670 | 3684 |
| 4894 | 3880 |
| 5291 | 8642 |
| 5408 | 13323 |
| 5434 | 8297 |
| 5501 | 3744 |
| 7458 | 8952 |
| 8790 | 6745 |
| 8896 | 8351 |
| 9012 | 17869 |
| 9062 | 8306 |
| 9155 | 5368 |
| 13249 | 8860 |
| 15325 | 9496 |
| 17385 | 17566 |
| 17483 | 8403 |
| 18650 | 5848 |
| 18691 | 8407 |
| 22823 | 8126 |
| 23017 | 6532 |
| 23435 | 7402 |
| 23447 | 7268 |
| 26693 | 7944 |
| 27469 | 6228 |
| 27496 | 8692 |
| 27744 | 13215 |
| 28143 | 7701 |
| 28936 | 7075 |
| 29835 | 5645 |
| 31024 | 8682 |
| 31061 | 7488 |
| 31342 | 8015 |
| A1 | 3661 |
| A10 | 4820 |
| A2 | 6551 |
| A4 | 4015 |
| A5 | 7042 |
| A6 | 3981 |
| A7 | 9735 |
| A8 | 7537 |
| A9 | 6256 |
| B1 | 10690 |
| B10 | 5862 |
| B2 | 11130 |
| B3 | 4653 |
| B4 | 8667 |
| B5 | 8898 |
| B6 | 8834 |
| B7 | 8808 |
| B8 | 7544 |
| B9 | 9040 |
| C3 | 8082 |
| C4 | 10549 |

TABLE 10
OTU Table Statistical Values

| Data | Values |
|---|---|
| Num samples: 63 | 63 |
| Num observations: | 8780 |
| Total count: 519923 | 519923 |
| Table density (fraction | 0.044 |
| of non-zero values): | |
| Min: | 3487.0 |
| Max: | 19245.0 |

Systemic inflammatory events and localized disease, mediated by the microbiome, may be measured in saliva as head and neck squamous cell carcinoma (HNSCC) diagnostic and prognostic biomonitors. We compared the saliva microbiome in DNA isolated from 38 patients and 25 normal oral cavity epithelium controls to characterize the HNSCC microbiota before and after surgical resection.

PCR amplification of the 16S rRNA V3-V5 gene region was performed using the 357F/926R primer set prior to multiplexing on the Roche/454 GS Junior sequencing platform. Data were screened for chimeric sequences and contaminant chloroplast DNA after pre-processing. Passing sequences were characterized for diversity and taxonomic composition using QIIME and R before cross-tabulation analyses were performed.

After preprocessing 142,887 reads were obtained with an average length of 491 bp. The number of sequences per sample was rarefied at 3,487 to guarantee equal depth. Bacteroidetes, Firmicutes, and Proteobacteria dominated the microbiome in our sample set with less frequent presence of Actinobacteria and Fusobacteria members. At lower taxonomic levels, the most abundant genera observed were Streptococcus, Prevotella, Haemophilus and Veillonella with lower numbers of Citrobacter and Neisseraceae genus Kingella.

We found that 46 OTUs changed significantly in HNSCC patients (p<0.05) when compared to the controls mainly due to the loss of Neisseria and Aggregatibacter (Proteobacteria), Leptotrichia (Fusobacteria) and Veilonella (Firmicutes) with an increase in some Lactobacillus (Firmicutes). Within bacteroidetes, Prevotella OTUs were found more abundant in control samples. HNSCC patients had a significant loss in richness and diversity (p<0.05) compared to the controls. HPV positive samples were more diverse (higher Shannon values and richness) than HPV negative samples.

Longitudinal analyses (3 time periods) of samples taken before and after surgery revealed a reduction in the alpha diversity measure after surgery, together with an increase of this measure in patients that recurred. We also observed statistically significant differences (p<0.05) at the phyla (Actinobacteria and Fusobacteria), and genus (Veillonella and Prevotella) levels. Interestingly, in one patient whose HPV status shifted from HPV positive to HPV negative after surgery, the abundance of Lactobacillus OTUs decreased, and Streptococcus (OTU 1009) increased significantly, being also associated with an HPV negative status in another patient.

We are the first to observe that OTUs and several microbial communities at different taxonomic levels discriminate HNSCC from control samples; HPV positive and HPV negative samples; and pre- vs postsurgical treatment samples. Future work will determine the correlation of microbial communities in paired tissue and saliva HNSCC samples, as well as their link to treatment response and survival.

1. A method for identifying a human subject as having head and neck squamous cell cancer (HNSCC) comprising the steps of:
a. obtaining nucleic acid from a saliva sample taken from the subject;
b. amplifying the 16S rRNA V3-V5 gene region of bacterial nucleic acid present in the nucleic acid of step (a);
c. sequencing the amplified DNA of step (b);
d. identifying the taxonomic levels of bacteria present in the saliva sample based on the sequences of step (c) and a comparison to taxonomic levels of bacteria present in a reference or control sample that correlates to normal mucosa; and
e. identifying the subject as having HNSCC or normal mucosa based on one or more of the following:
i. enrichment in the observed species category of alpha-diversity estimators is indicative of normal mucosa;
ii. enrichment of Bacteriodetes Flavobacteria is indicative of normal mucosa;
iii. the presence of the genus Tannerella in the sample is indicative of normal mucosa;
iv. enrichment of Fusobacteriales Leptotrichiaceae is indicative of normal mucosa;
v. a higher number of operational taxonomic units (OTUs) is indicative of normal mucosa;
vi. a lower level of Aerococcaceae Abiotrophia is indicative of normal mucosa;
vii. at the family level, the taxon Fusobacteriales Leptotrichiaceae is dramatically enriched in normal mucosa;
viii. a threshold of 10 sequences assigned to Fusobacteriales Leptotrichiaceae distinguishes HNSCC from normal mucosa;
ix. the genus Tannerella was exclusively observed in normal mucosa;
x. threshold of 80 OTUs was enough to perfectly distinguish UPPP from OPSCC samples;
xi. 46 OTUs changed significantly in HNSCC patients (p<0.05) when compared to the controls mainly due to the loss of Neisseria and Aggregatibacter (Proteobacteria), Leptotrichia (Fusobacteria) and Veilonella (Firmicutes) with an increase in some Lactobacillus (Firmicutes); and
xii. within Bacteroidetes, Prevotella OTUs were found more abundant in control samples.

2. The method of claim 1, wherein the primer set comprises the 357F/296R primer set.

3. The method of claim 1, wherein the primer set comprises SEQ ID NO:2 and SEQ ID NO:3.

4. The method of claim 1, wherein a threshold of 80 OTUs is used in step (e)(v) to distinguish between normal from HNSCC.

5. The method of claim 1, wherein the alpha-diversity estimators of step (e)(i) comprises Chao2, ACE, Shannon and Simpson index.

6. The method of claim 1, wherein a threshold of 10 sequences is used in step (e)(iv) to determine enrichment of Fusobacteriales Leptotrichiaceae.

7. The method of claim 1, wherein the subject is human papillomavirus (HPV) positive.

8. The method of claim 1, wherein the subject is HPV negative.

9. The method of claim 1, further comprising treating the subject with an appropriate treatment modality for HSNCC.

10. The method of claim 9, wherein the treatment modality is one or more of surgery, radiotherapy, and chemotherapy.

11. The method of claim 10, wherein the treatment modality further comprises one or more of administering a cell cycle inhibitor, a PI3K inhibitor and/or a mTOR inhibitor.

12. The method of claim 1, wherein the subject is identified as having HNSCC and further comprising the step of:
f. identifying the HNSCC subject as HPV+ or HPV− based on one or more of the following:
i. at the class level, statistical depletion of Bacteroidetes Flavobacteria in HNSCC HPV+ samples relative to control samples;
ii. at the genus level, low-level presence of Aerococcaceae Abiotrophia distinguishes normal mucosa from OPSCC HPV+ samples;
iii. HPV+ samples are more diverse in terms of phylum, having unique Chloroflexi, Proteobacterial and Prevotella OTUs;
iv. HPV− samples have unique Actinobacterial OTUs that are lacking in the HPV+ samples; and
v. HPV+ samples enriched in the observed species category of alpha-diversity estimators relative to control samples.

13. The method of claim 12, wherein the unique Actinobacterial OTUs of step (f)(iv) comprise Bifidobacteriaceae.

14. The method of claim 12, wherein the alpha-diversity estimators of step (f)(v) comprise Chao1, ACE, Shannon and Simpson index.

15. The method of claim 12, further comprising the step of treating the subject with an appropriate treatment modality for HSNCC.

16. The method of claim 15, wherein the treatment modality is one or more of surgery, radiotherapy, and chemotherapy.

17. The method of claim 15 or 16, wherein the treatment modality comprises administering a cell cycle inhibitor to a HNSCC HPV− subject.

18. The method of claim 15 or 16, wherein the treatment modality comprises administering a PI3K inhibitor and/or a mTOR inhibitor to a HNSCC HPV+ subject.


SEQUENCE LISTING (14 sequences)

SEQ ID NO: 1 - DNA, 10 bp, Artificial Sequence
  Barcode sequence
acgagtgcgt

SEQ ID NO: 2 - DNA, 18 bp, Artificial Sequence
  Primer
ccgtcaattcmtttragt

SEQ ID NO: 3 - DNA, 17 bp, Artificial Sequence
  Primer
cctacgggaggcagcag

SEQ ID NO: 4 - DNA, 10 bp, Artificial Sequence
  Barcode sequence
acgctcgaca

SEQ ID NO: 5 - DNA, 10 bp, Artificial Sequence
  Barcode sequence
agacgcactc

SEQ ID NO: 6 - DNA, 10 bp, Artificial Sequence
  Barcode sequence
agcactgtag

SEQ ID NO: 7 - DNA, 10 bp, Artificial Sequence
  Barcode sequence
atcagacacg

SEQ ID NO: 8 - DNA, 10 bp, Artificial Sequence
  Barcode sequence
atatcgcgag

SEQ ID NO: 9 - DNA, 10 bp, Artificial Sequence
  Barcode sequence
cgtgtctcta

SEQ ID NO: 10 - DNA, 10 bp, Artificial Sequence
  Barcode sequence
ctcgcgtgtc

SEQ ID NO: 11 - DNA, 10 bp, Artificial Sequence
  Barcode sequence
acgctcgaca

SEQ ID NO: 12 - DNA, 10 bp, Artificial Sequence
  Barcode sequence
agacgcactc

SEQ ID NO: 13 - DNA, 10 bp, Artificial Sequence
  Barcode sequence
agcactgtag

SEQ ID NO: 14 - DNA, 10 bp, Artificial Sequence
  Barcode sequence
ctcgcgtgtc